(19) United States
(12) Patent Application Publication
(10) Pub. No.: US 2017/0362388 A1
(43) Pub. Date: Dec. 21, 2017

(54) Disulfide Polymers and Methods of Use

(71) Applicant: The Brigham and Women's Hospital, Inc., Boston, MA (US)

(72) Inventors: Omid C. Farokhzad, Waban, MA (US); Jun Wu, Boston, MA (US); Lili Zhao, Nanjing (CN)

(21) Appl. No.: 15/510,597

(22) PCT Filed: Sep. 11, 2015
(86) PCT No.: PCT/US15/49702

§ 371 Date: Mar. 10, 2017

Related U.S. Application Data
(60) Provisional application No. 62/049,117, filed on Sep. 11, 2014.
(60) Provisional application No. 62/182,178, filed on Jun. 19, 2015.

(51) Int. Cl. C08G 75/14; A61K 47/59; A61K 9/50; A61K 31/704

(52) U.S. Cl. CPC C08G 75/14; A61K 31/704; A61K 47/59; A61K 9/5031

(57) ABSTRACT

Provided herein are disulfide polymers, methods of their preparation, compositions, and methods of using the same. For example, provided herein are polymers having at least one repeating unit according to Formula (I):or Formula (II): and nanoparticles and compositions including the same. The polymers and compositions provided herein may be used, for example, in the encapsulation of drugs for a slow release drug administration in the treatment or imaging of diseases.

### CROSS-REFERENCE TO RELATED APPLICATIONS

This application claims the benefit of U.S. Provisional Application No. 62/049,117, filed Sep. 11, 2014, and U.S. Provisional Application No. 62/182,178, filed Jun. 19, 2015, each of which is incorporated herein by reference in its entirety.

### STATEMENT AS TO FEDERALLY SPONSORED RESEARCH

This invention was made with Government support under Grant Nos. EB015419 and CA151884, awarded by the National Institutes of Health. The Government has certain rights in the invention.

### TECHNICAL FIELD

This invention relates to disulfide (e.g., cystine, cysteine) polymer compositions and polymer complexes.

### BACKGROUND

Disulfide based materials, particularly cystine/cysteine based materials, have sparked interest due to their biocompatibility and abundance in nature. However, there is limited information available for cystine/cysteine based materials and their applications.

Nanoscale systems, such as nanoparticles, have been used to encapsulate species, for example, to enhance delivery to a target or to protect a species from degradation in an unfavorable environment. Nanoparticles offer the ability, e.g., to encapsulate species that can later be released to afford a desired effect. For example, nanoparticles can be used for gene delivery or in cancer therapy.

### SUMMARY

Disulfide based materials are of interest due to their sensitivity to a reducing environment. For example, in nanomedicine, disulfide nanoparticles can be used to encapsulate species such as small molecule cancer drugs in order to protect them from degradation before delivery to the target. Encapsulation methods allow for a prolonged half-life of, e.g., a cancer drug, thus providing the ability for more effective treatment at a lower dose of the drug. However, disulfide nanoparticles can suffer from difficult syntheses and characteristics that limit the loading of desired species. For example, many disulfide systems cannot effect a high loading of hydrophobic cancer drugs due to their hydrophilicity. Another issue is that many disulfide systems are lack of functional groups to load charged drug molecules or for further modifications or conjugations. The present disclosure provides methods and compositions of disulfide polymer platforms, e.g., hydrophobic disulfide polymers and corresponding nanoparticles that can be prepared simply, rapidly, and under mild conditions, and that offer high loading of hydrophobic moieties, such as cancer drugs. In one embodiment, the present invention provides methods for the synthesis of disulfide based polymers and for preparing drug delivery compositions, e.g., nanoparticles, that include active agents encapsulated by polymers, e.g., cysteine-based polymers.

Provided herein is a polymer comprising at least one repeating unit according to Formula (I) or Formula (II):

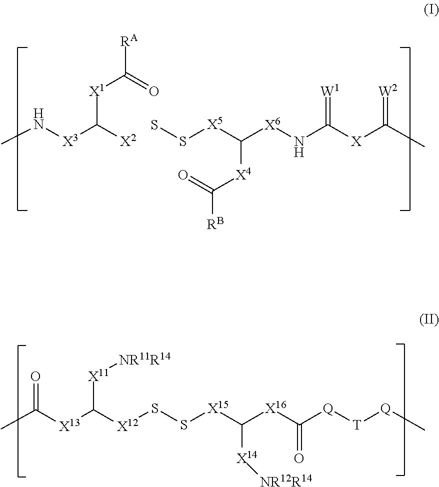

wherein:

X¹is a bond or C₁₋₁₀₀ alkylene;

X² is C₁₋₁₀₀ alkylene;

X³ is a bond or C₁₋₁₀₀ alkylene;

X⁴ is a bond or C₁₋₁₀₀ alkylene;

X⁵ is C₁₋₁₀₀ alkylene;

X⁶ is a bond or C₁₋₁₀₀ alkylene;

R^{A} is OR¹ or NR¹R⁴;

R^{B} is OR² or NR²R⁴;

R¹ is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₁₀₀ alkyl or C(═O)R⁶;

each R⁴ is independently H or C₁₋₁₀₀ alkyl;

each R⁵ is independently H or C₁₋₁₀₀ alkyl;

each R⁶ is independently H or C₁₋₁₀₀ alkyl;

W¹ is O, S, or NH;

W² is O, S, or NH;

X is C₁₋₁₀₀ alkylene, C₂₋₁₀₀ alkenylene, or C₂₋₁₀₀ alkynylene;

provided that when W¹ and W² are both O, then X is C₃₋₁₀₀ alkylene, C₂₋₁₀₀ alkenylene, or C₂₋₁₀₀ alkynylene;

each m is 0, 1 or 2;

X¹¹ is a bond or C₁₋₁₀₀ alkylene;

X¹² is C₁₋₁₀₀ alkylene;

X¹³ is a bond or C₁₋₁₀₀ alkylene;

X¹⁴ is a bond or C₁₋₁₀₀ alkylene;

X¹⁵ is C₁₋₁₀₀ alkylene;

X¹⁶ is a bond or C₁₋₁₀₀ alkylene;

R¹¹ is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹¹ is optionally substituted with 1, 2, or 3 substituents independently selected from the group consisting of: halo, —CN, OR¹³, NR¹³R¹⁴, —(C═O)R¹⁴,
—(C═O)OR¹⁴, —(C═O)NR¹⁴R¹⁵, —S(O)R¹⁴, and C₆₋₁₀ aryl;

R¹² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹² is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;

each R¹³ is independently H, C₁₋₁₀₀ alkyl or C(═O)R¹⁶;

each R¹⁴ is independently H or C₁₋₁₀₀ alkyl;

each R¹⁵ is independently H or C₁₋₁₀₀ alkyl;

each R¹⁶ is independently H or C₁₋₁₀₀ alkyl;

each Q is independently O or NR¹⁷;

each R¹⁷ is H or C₁₋₁₀₀ alkyl;

T is C₂₋₁₀₀ alkylene, C₄₋₁₀₀ alkenylene, or C₄₋₁₀₀ alkynylene; and

each n is 0, 1 or 2.

In some embodiments, the polymer comprises at least one repeating unit according to Formula (Ia):

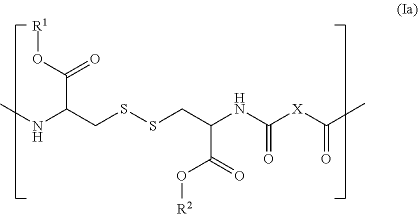

wherein:

R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₆ alkyl or C(═O)R⁶;

each R⁴ is independently H or C₁₋₆ alkyl;

each R⁵ is independently H or C₁₋₆ alkyl;

each R⁶ is independently H or C₁₋₆ alkyl;

X is C₃₋₂₀ alkylene, C₂₋₂₀ alkenylene, or C₂₋₂₀ alkynylene; and

each m is 0, 1 or 2.

Provided herein is a salt form of a polymer as described herein. In some embodiments, the salt form of a polymer comprises a metal counterion.

Also provided herein is a nanoparticle comprising a polymer as described herein.

Also provided herein is a method of preparing a nanoparticle as described herein, comprising dissolving a polymer as described herein in a polar aprotic solvent to give a polymer solution; and adding the polymer solution to water to provide the nanoparticle.

Provided herein is a composition comprising a polymer as described herein and a drug molecule, or a pharmaceutically acceptable salt thereof.

Also provided herein is a composition comprising a polymer as described herein and a detectable agent, or a pharmaceutically acceptable salt thereof.

Provided herein is a method of transporting a drug molecule into a cell, comprising contacting the cell with a composition as described herein.

Provided herein is a method of treating a disease or condition in a subject in need thereof, comprising administering to the subject a therapeutically effective amount of a polymer, a nanoparticle, or a composition as described herein.

Also provided herein is a method of imaging a disease or condition in a subject, comprising administering to the subject a composition as described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the description and drawings, and from the claims.

### BRIEF DESCRIPTION OF THE DRAWINGS

FIG. 1 is a scheme depicting the use of cysteine-based polymer nanoparticles for therapeutically delivering, e.g., a hydrophobic drug, e.g., to a cancer cell.

FIGS. 2A-2D are exemplary NMR spectra of cysteine polymers. FIG. 2A is the ¹H NMR spectrum of cysteine dicarboxylate copolymer with sebacoyl chloride (Cys-OH-8) as the triethylamine salt in DMSO-d6. FIG. 2B is the ¹³C NMR spectrum of Cys-OH-8 as the triethylamine salt in DMSO-d6. FIG. 2C is the ¹H NMR spectrum of Cys-OH-8 as the free acid in DMSO-d6. FIG. 2D is the ¹³C NMR spectrum of Cys-OH-8 as the free acid in DMSO-d6. FIGS. 2E to 2I are exemplary DSC plots for the cysteine polymers. FIG. 2E is a DSC plot for Cys-OMe-2/Cys-2E polymer. FIG. 2F is a DSC plot for Cys-OMe-4/Cys-4E polymer. FIG. 2G is a DSC plot for Cys-OMe-6/Cys-6E polymer. FIG. 2H is a DSC plot for Cys-OMe-8/Cys-8E polymer. FIG. 21 is a DSC plot for Cys-OMe-10/Cys-10E polymer. FIGS. 2J-2M are exemplary NMR spectra of cysteine polymers. FIG. 2J is the ¹H NMR spectrum of Cys-OMe-2/Cys-2E polymer in DMSO-d6. FIG. 2K is the ¹H NMR spectrum of Cys-OMe-4/Cys-4E polymer in DMSO-d6. FIG. 2L is the ¹H NMR spectrum of Cys-OMe-6/Cys-6E polymer in DMSO-d6. FIG. 2M is the ¹H NMR spectrum of Cys-OMe-8/Cys-8E polymer in DMSO-d6.

FIG. 3 is a chromatogram of cysteine dicarboxylate copolymer with suberoyl chloride (Cys-OH-6) using aqueous buffer as solvent.

FIG. 4 is a plot of the size distribution of cysteine methyl ester nanoparticles as determined by DLS in deionized water at room temperature: y-axis is DLS % intensity; x-axis is nanoparticle size (nm). Curves: (a) Cys-OMe-2/Cys-2E NPs; (b) Cys-OMe-4/Cys-4E NPs; (c) Cys-OMe-6/Cys-6E NPs; (d) Cys-OMe-8/Cys-8E NPs; (e) Cys-OMe-10/Cys-10E NPs.

FIG. 5A-5S are TEM images of nanoparticles comprising cysteine. FIG. 5A is an image of nanoparticles of cysteine dimethyl ester copolymer with sebacoyl chloride (Cys-OMe-8/Cys-8E NPs). White bar at lower right indicates 100 nm. FIG. 5B is an image of Cys-OH-8 triethylamine salt NP formed in the presence of fingolimod (1:1 molar ratio) at 0.1 mg/mL concentration. FIG. 5C is an image of Cys-OH-8 triethylamine salt NP formed in the presence of FeCl2 (8:3 wt/wt ratio [left]; 2:1 wt/wt ratio [right]). FIG. 5D is an image of Cys-OH-8 triethylamine salt NP formed in the presence of FeCl2 (8:3 wt/wt ratio [left]), and Cys-OH-6 triethylamine salt NP formed in the presence of FeCl2 (2:1 wt/wt ratio [right]). FIG. 5E is an image of Cys-OH-10 triethylamine salt NP formed in the presence of CuCl2 (4:1 wt/wt ratio). FIG. 5F is an image of Cys-OH-8 triethylamine salt NP formed in the presence of AgNO3 (2:1 wt/wt ratio). Left image with no Ag stain; right image with Ag stain. FIG. 5G is an image of cysteine polymer triethylamine NPs formed in the presence of Li⁻ salt: left: Cys-OH-4; right: Cys-OH-8. FIG. 5H is an image of two views of Cys-OH-4 triethylamine NPs formed in the presence of Gd³⁺ salt. FIG. 51 is an image of salt free complex of Cys-OH-8 NP formed in the presence of doxorubicin (1:1 ratio) as nanospheres. FIG. 5J is an image of Cys-OMe-8/Cys-8E NPs loaded with 10 wt % docetaxel (Dtxl). FIG. 5K is an image of Cys-OH-8 NP triethylamine salt formed in the presence of doxorubicin hydrochloride (1:1 ratio) as nanorods. FIG. 5L is a cross section of Cys-OH-8 NP triethylamine salt formed in the presence of doxorubicin hydrochloride (1:1 ratio) as nanorods. Diameter is 50-80 nm. FIG. 5M is an image with detail of Cys-OH-8 NP triethylamine salt formed in the presence of doxorubicin hydrochloride (1:1 ratio) as nanorods with an aligned fiber structure. The distance between the fibers is about 2.5-3.5 nm. FIG. 5N is an image of Cys-OH-8 NP triethylamine salt formed in the presence of doxorubicin hydrochloride (2:1 ratio) with a nanofiber structure. FIG. 5O is an image of Cys-OH-6 NP triethylamine salt formed in the presence of doxorubicin hydrochloride (1:1 molar ratio). FIG. 5P is an image of Cys-OH-8 triethylamine salt formed in the presence of doxorubicin hydrochloride (1:1 molar ratio) with DSPE-PEG3000/Lipid coating. FIG. 5Q is an image of Cys-OH-8 triethylamine salt formed in the presence of doxorubicin hydrochloride (1:1 molar ratio) with PLGA50K/DSPE-PEG3000 coating. FIG. 5R is an image of Cys-OH-10 triethylamine salt formed in the presence of doxorubicin hydrochloride (1:1 molar ratio). FIG. 5S is an image of Cys-OH-8 free acid formed in the presence of doxorubicin *0.5 hydrochloride (1:1 molar ratio).

FIG. 6 is a plot of the particle size changes of Cys-8E/DSPE-PEG-3000 in 1× PBS buffer and 10% FBS DMEM cell culture medium over one week.

FIGS. 7A and B are plots depicting fluorescence intensity changes of various cysteine nanoparticles (NPs) loaded with Nile red over time upon incubation in the indicated buffers. FIG. 7A depicts the fluorescence intensity change (y-axis)-time (x-axis) curve for different cysteine NPs. Curves: (a) Cys-OMe-4/ Cys-4E NPs in PBS; (b) Cys-OMe-4/Cys-4E NPs in DTT; (c) Cys-OMe-6/Cys-6E NPs in PBS; (d) Cys-OMe-6/Cys-6E NPs in DTT; (e) Cys-OMe-8/Cys-8E NPs in PBS; (f) Cys-OMe-8/Cys-8E NPs in DTT; (g) Cys-OMe-10/Cys-10E NPs in PBS; (h) Cys-OMe-10/Cys-10E NPs in DTT. FIG. 7B depicts the fluorescence intensity change (y-axis)-time (x-axis) curve for Cys-OMe-8/Cys-8E NPs loaded with Nile red upon incubation in the indicated buffers. Curves: (a) in PBS buffer only; (b) 10 mM DTT; (c) 5 mM DTT; (d) 1 mM DTT; (e) 0.1 mM DTT; (f) 10 mM DTT at pH 5.

FIG. 8 is a plot of the particle size change (y-axis)-time (x-axis) curve for Cys-OMe-8 NPs as measured by DLS. Curves: small diamonds: PBS only; squares: PBS+1 mM DTT; triangles: PBS+10 mM DTT.

FIG. 9 is a TEM images of Cys-OMe-8 NPs after treatment with DTT.

FIG. 10 shows TEM images of dissembled Cys-8E/ Cys-8E NPs indicating second self-assembly (Scale bar: A-B, 2 μm; C-D, 10 μm).

FIG. 11 is a GPC profile of Cys-OMe-8/Cys-8E before and after 10 mM GSH treatment.

FIG. 12 is a plot of cell viability of Hela cells after treatment with various concentrations (15.63 μg/mL, 31.25 μg/mL and 62.5 μg/mL) of Cys-OMe-8/Cys-8E nanoparticles (concentration unit: μg/mL).

FIG. 13 are images showing fluorescence resonance energy transfer (FRET) effect for DSPE-PEG-coated Cys-OMe-8/Cys-8E NPs with 0.09 wt % Coumarin 6 and 0.91 wt % Nile Red co-encapsulated.

FIG. 14 is a plot showing Cys-OH-8 triethylamine salt with doxorubicin nanorod drug release. Square (PBS-7) is PBS buffer at pH 7; circle is 10 mM citrate buffer at pH 5; triangle is 10 mM phosphate buffer at pH 5; inverted triangle is 100 mM citrate buffer at pH 5; diamond is 100 mM acetate buffer at pH 5.

FIG. 15 is a plot showing Cys-OH-8 triethylamine salt with doxorubicin nanorod drug release with detergent. Square (PBS) is PBS buffer only; circle is PBS with 0.1% TRITON® ×100; triangle is PBS with 1% TRITON® ×100; inverted triangle is at pH 5 with 0.1% TRITON® ×100.

FIG. 16 is a plot showing Cys-OH-8 triethylamine salt with doxorubicin nanorod drug release under reductive conditions. Square (PBS) is PBS buffer only; circle is PBS with 1 mM DTT; triangle is PBS with 10 mM DTT; inverted triangle is at pH 5; diamond is at pH 5 with 10 mM DTT.

FIG. 17 is a plot showing effects of DSPE-PEG-coated Cys-OMe-8 NPs with 10 wt % docetaxel encapsulated against cancer cells. Hela and DU145 cells were treated with docetaxel-loaded Cys-OMe-8 NPs for 4 h or 48 h. Cells treated with free docetaxel were used as controls.

FIG. 18A and 18B are images showing the fluorescence resonance energy transfer (FRET) effect for Cys-OMe-8-Cys-8E/DSPE-mPEG3000 hybrid NPs with 0.1 wt % Coumarin 6 and 1 wt % Nile Red. For FIG. 18A, A549 cells were used and pretreated with 50 μM nethylmaleimide (NEM) for 1 h to inhibit GSH. The image was recorded after 4 h. FIG. 18B shows an image taken without NEM treatment, intracellular performance was recorded after 4 h cell-uptake of NPs.

FIG. 19 is a compilation of CLSM images of A549 cells incubated with Cys-Ome-8/Cys-8E NPs for 1 h (A₁-A₄) and 4 h (B₁-B₄). The nuclei, endosomes, and NPs were stained using Hoechst 33342 (A₁, B₁), lysotracker green (A₂, B₂), and Dil (A₃, B₃), respectively.

FIG. 20A and 20B are plots showing effects on cell viability of A549 cells treated with Cys-OH-8 NPs containing doxorubicin. FIG. 20A is a plot showing effects after 4 h treatment; FIG. 20B is a plot showing effects after 48 h treatment. Squares (DOX)=only doxorubicin treatment; circles (NPs-ROD)=Cys-OH-8 nanorods containing doxorubicin; triangles (NPs-SPHERE)=Cys-OH-8 nanospheres containing doxorubicin; inverted triangles (LPs)=liposomes containing doxorubicin.

FIG. 21A and 21B are plots showing effects on cell viability of H460 cells treated with Cys-OH-8 NPs containing doxorubicin. FIG. 21A is a plot showing effects after 4 h treatment; FIG. 21B is a plot showing effects after 48 h treatment. Squares (DOX) =only doxorubicin treatment; circles (NPs-ROD)=Cys-OH-8 nanorods containing doxorubicin; triangles (NPs-SPHERE)=Cys-OH-8 nanospheres containing doxorubicin; inverted triangles (LPs)=liposomes containing doxorubicin.

FIG. 22 is a pair of plots showing the in vivo pharmacokinetics of Cys-OH-8 NPs containing doxorubicin in A549 tumor mice for a single 5 mg/kg iv dose. y-axis: plasma doxorubicin concentration; x-axis: time. Left graph shows data after 8 h; right graph shows data after 96 h. Squares (DOX)=only doxorubicin treatment; circles (NPs-ROD)=Cys-OH-8 nanorods containing doxorubicin; triangles (NPs-SPHERE)=Cys-OH-8 nanospheres containing doxorubicin; inverted triangles (LPs)=liposomes containing doxorubicin.

FIG. 23 shows in vivo biodistribution data for Cys-OH-8 NPs containing doxorubicin in A549 tumor mice for a single 5 mg/kg iv dose 48 h post-injection. For each organ, 1^{st} bar shows DOX (doxorubicin only); 2^{nd} bar shows NPs-ROD (Cys-OH-8 nanorods containing doxorubicin); 3^{rd} bar shows NPs-SPHERE (Cys-OH-8 nanospheres containing doxorubicin); 4^{th} bar shows LPs (liposomes containing doxorubicin).

FIGS. 24A to 24D are plots showing the anticancer effects of docetaxel-loaded Cys-OMe-8/Cys-8E NPs in Hela and DU145 cells: In FIGS. 24A and 24C: HeLa cells were treated with docetaxel-loaded NPs for 4 h and 48 h, respectively. In FIGS. 24B and 24D: DU145 cells were treated with docetaxel-loaded NPs for 4 h and 48 h, respectively.

FIG. 25 is a plot of cell viability after treatment for 4 h with medium (control), free docetaxel (Dtxl), docetaxel-loaded Cys-8E NPs, and docetaxel loaded Cys-8E NPs and NEM (docetaxel concentration: 500 ng/mL).

FIG. 26 is a plot of the relative tumor size in A549 mice after treatment of 5 mg/kg iv dose for two weeks. Triangle=only PBS; square=(DOX) only doxorubicin; left-facing triangle (CYS-8C): only CYS-OH-8 NP (Et3N salt), no drug; circle=(LPs) liposome containing doxorubicin; inverted triangle=(NPs-Rod): CYS-OH-8 containing doxorubicin nanorods; diamond=(NPs-Sphere) CYS-OH-8 containing doxorubicin nanospheres.

FIG. 27 is a plot of the relative tumor volume in A549 mice after treatment of 5 mg/kg iv dose for two weeks. Triangle=only PBS; square=(DOX) only doxorubicin; left-facing triangle (CYS-8C): only CYS-OH-8 NP (Et3N salt), no drug; circle=(LPs) liposome containing doxorubicin; inverted triangle=(NPs-Rod): CYS-OH-8 containing doxorubicin nanorods; diamond=(NPs-Sphere) CYS-OH-8 containing doxorubicin nanospheres.

FIG. 28 is a plot of the relative tumor size vs. days post tumor transplantation in A549 tumor mice with different doses of indicated material. Square=PBS only; top circles=Cys-OH-8 triethylamine NP only, no drug, at 20 mg/kg; triangle=doxorubicin at 15 mg/kg dose; inverted triangle=doxorubicin at 5 mg/kg dose; diamond=liposomes with 5 mg/kg doxorubicin; left-facing triangle=liposomes with 15 mg/kg doxorubicin; right-facing triangle=Cys-OH-8 triethylamine NP containing 5 mg/kg doxorubicin; bottom circles=Cys-OH-8 triethylamine NP containing 15 mg/kg doxorubicin.

FIG. 29 is a plot of the relative tumor volume vs. days post tumor transplantation in A549 tumor mice with different doses of indicated material. Square=PBS only; top circles=Cys-OH-8 triethylamine NP only, no drug, at 20 mg/kg; triangle=doxorubicin at 15 mg/kg dose; inverted triangle=doxorubicin at 5 mg/kg dose; diamond=liposomes with 5 mg/kg doxorubicin; left-facing triangle=liposomes with 15 mg/kg doxorubicin; right-facing triangle=Cys-OH-8 triethylamine NP containing 5 mg/kg doxorubicin; bottom circles=Cys-OH-8 triethylamine NP containing 15 mg/kg doxorubicin.

FIG. 30 is a plot showing the effect of up to 10 wt % docetaxel-loaded Cys-OMe-8/Cys-8E NPs on tumor volume (mm³) in A549 tumor mice over two weeks. Square=PBS only; circles=docetaxel-loaded Cys-OMe-8/Cys-8E NP low dose at 5 mg/kg; triangle=docetaxel-loaded Cys-OMe-8 NP high dose at 10 mg/kg; inverted triangle=docetaxel-loaded PLGA NPs at 5 mg/kg; diamonds=docetaxel alone at 5 mg/kg.

FIG. 31 is a plot showing the effect on mouse weight after treatment with control (PBS) (squares), (ii) docetaxel at 5 mg/kg (inverted triangles), (iii) Cys-OMe-8/Cys-8E NPs at a docetaxel equivalent dose of 5 mg/kg (“Cys-8E-L”) (circles), (iv) Cys-OMe-8/Cys-8E NPs at a docetaxel equivalent dose of 10 mg/kg (“Cys-8E-H”) (triangles).

FIG. 32A is a collection of fluorescent images of the tumors and main organs of A549 tumor-bearing nude mice sacrificed at 2 h, 24 h, or 48 h post-injection of the NPs made with Cys-8E; FIG. 32B is a collection of fluorescent images of the tumors and main organs of A549 tumor-bearing nude mice sacrificed at 24 h post-injection of the NPs made with PLGA. FIG. 32C is a plot of the quantitative biodistribution profiles of NPs in the tumors and main organs of A549 tumor-bearing nude mice sacrificed at 24 h post-injection of the NPs made with Cys-8E or PLGA.

FIG. 33 is a plot of the pharmacokinetic profiles obtained for docetaxel-loaded PLGA NPs and Cys-8E NPs.

FIG. 34 is a collection of images of histological sections of the major organs (A: heart; B: liver; C: spleen; D: lung; E: kidney) of mice given intravenous injections of PBS (A₁-E₁) or Cys-8E NPs (A₂-E₂) with hematoxylin-eosin staining and 100× magnification.

### DETAILED DESCRIPTION

The current application provides disulfide-based biodegradable and biocompatible polymers that exhibit hydrophobic or functional properties. In one aspect, provided is a cysteine copolymer with a dicarboxylic acid, or derivative thereof, methods of preparation, compositions, and methods of use. In another aspect, provided is a cysteine copolymer with a diamine or diol, or derivative thereof, methods of preparation, compositions, and methods of use. In some embodiments, the hydrophobic disulfide polymers self-assemble in an aqueous liquid to form water-insoluble nanoparticle compositions, which can afford a high degree of loading, e.g., of hydrophobic cancer chemotherapeutics. In some embodiments, the acidic or cationic disulfide polymers self-assemble with drugs and biomolecules in an aqueous liquid to form water-insoluble nanoparticle compositions, which can afford a high degree of loading, e.g., of protein, peptide, nucleic acids and chemotherapeutics. In some embodiments, the polymer and drugs can also self-assemble in organic solvents, form nanoparticle structures in aqueous solutions, or after drying, or any other possible conditions. Such compositions can be delivered in a controlled release fashion to provide enhanced delivery of a cancer or other chemotherapeutic with an improved in vivo half-life compared to the free drug. In another non-limiting example, cationic or anionic disulfide systems could be prepared simply, rapidly, and under mild conditions. Such systems offer high loading of drug moieties, such as proteins, peptides, nucleic acids for cationic systems, and cationic drugs, such as anticancer and antibiotic drugs, for anionic systems.

In the present description, it is appreciated that certain features described herein, which are, for clarity, described in the context of separate embodiments, can also be provided in combination in a single embodiment. Conversely, various features described herein which are, for brevity, described in the context of a single embodiment, can also be provided separately or in any suitable subcombination.

Although methods and materials similar or equivalent to those described herein can be used in the practice or testing of the present invention, suitable methods and materials are described below. In addition, the materials, methods and examples are illustrative only and not intended to be limiting.

### I. Definitions

Unless defined otherwise, all technical and scientific terms used herein have the same meaning as is commonly understood by one of ordinary skill in the art to which this disclosure belongs.

As used herein, bonds symbolized by a simple line do not indicate a stereo-preference. Unless otherwise indicated to the contrary, chemical structures, which include one or more stereocenters, illustrated herein without indicating absolute or relative stereochemistry encompass all possible stereoisomeric forms of the polymer (e.g., diastereomers, enantiomers) and mixtures thereof Structures with a single bold or dashed line, and at least one additional simple line, encompass a single enantiomeric series of all possible diastereomers. In particular, the cysteine amino acids and analogs thereof included in the polymers described herein can have D, L, or D/L configuration.

For the terms “e.g.” and “such as,” and grammatical equivalents thereof, the phrase “and without limitation” is understood to follow unless explicitly stated otherwise.

As used herein, the singular forms “a,” “an,” and “the” include plural referents unless the context clearly dictates otherwise.

As used herein, the term “about” means “approximately” (e.g., plus or minus approximately 10% of the indicated value).

As used herein, “alkyl” refers to a saturated hydrocarbon chain that may be a straight chain or a branched chain. An alkyl group formally corresponds to an alkane with one C—H bond replaced by the point of attachment of the alkyl group to the remainder of the polymer. The term “(C_{x-y})alkyl” (wherein x and y are integers) by itself or as part of another substituent means, unless otherwise stated, an alkyl group containing from x to y carbon atoms. For example, a C₁₋₆)alkyl group may have from one to six (inclusive) carbon atoms in it. Examples of C₁₋₆)alkyl groups include, but are not limited to, methyl, ethyl, n-propyl, n-butyl, n-pentyl, n-hexyl, isopropyl, isobutyl, sec-butyl, tent-butyl, isopentyl, neopentyl and isohexyl. The (C_{x-y})alkyl groups include C₁₋₆)alkyl, (C₁₋₄)alkyl and (C₁₋₃)alkyl.

The term “(C_{x-y})alkylene” (wherein x and y are integers) refers to an alkylene group containing from x to y carbon atoms. An alkylene group formally corresponds to an alkane with two C—H bonds replaced by points of attachment of the alkylene group to the remainder of the polymer. Examples are divalent straight hydrocarbon groups consisting of methylene groups, such as, —CH₂—, —CH₂CH₂—, —CH₂CH₂CH₂—. The (C_{x-y})alkylene groups include C₁₋₆)alkylene and (C₁₋₃)alkylene.

As used herein, “alkenyl” refers to an unsaturated hydrocarbon chain that includes a C═C double bond. An alkenyl group formally corresponds to an alkene with one C—H bond replaced by the point of attachment of the alkenyl group to the remainder of the polymer. The term “(C_{x-y})alkenyl” (wherein x and y are integers) denotes a radical containing x to y carbons, wherein at least one carbon-carbon double bond is present (therefore x must be at least 2). Some embodiments are 2 to 4 carbons, some embodiments are 2 to 3 carbons and some embodiments have 2 carbons. Alkenyl groups may include both E and Z stereoisomers. An alkenyl group can include more than one double bond. Examples of alkenyl groups include vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2,4-hexadienyl, and the like.

The term “(C_{x-y})alkenylene” (wherein x and y are integers) refers to an alkenylene group containing from x to y carbon atoms. An alkenylene group formally corresponds to an alkene with two C—H bonds replaced by points of attachment of the alkenylene group to the remainder of the polymer. Examples are divalent straight hydrocarbon groups consisting of alkenyl groups, such as —HC═CH— and —HC═CH—CH₂—. The (C_{x-y})alkenylene groups include (C₂₋₆)alkenylene and (C₂₋₄)alkenylene. The term “(C_{x-y})heteroalkylene” (wherein x and y are integers) refers to a heteroalkylene group containing from x to y carbon atoms. A heteroalkylene group corresponds to an alkylene group wherein one or more of the carbon atoms have been replaced by a heteroatom. The heteroatoms may be independently selected from the group consisting of O, N and S. A divalent heteroatom (e.g., O or S) replaces a methylene group of the alkylene —CH₂—, and a trivalent heteroatom (e.g., N) replaces a methine group. Examples are divalent straight hydrocarbon groups consisting of methylene groups, such as, —CH₂—, —CHY₂CH₂—, —CH₂CH₂CH₂—. The (C_{x-y})alkylene groups include (C₁₋₆)heteroalkylene and (C₁₋₃)heteroalkylene.

As used herein, “alkynyl” refers to an unsaturated hydrocarbon chain that includes a C≡C triple bond. An alkynyl group formally corresponds to an alkyne with one C—H bond replaced by the point of attachment of the alkyl group to the remainder of the polymer. The term “(C_{x-y})alkynyl” (wherein x and y are integers) denotes a radical containing x to y carbons, wherein at least one carbon-carbon triple bond is present (therefore x must be at least 2). Some embodiments are 2 to 4 carbons, some embodiments are 2 to 3 carbons and some embodiments have 2 carbons. Examples of an alkynyl include ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

The term “(C_{x-y})alkynylene” (wherein x and y are integers) refers to an alkynylene group containing from x to y carbon atoms. An alkynylene group formally corresponds to an alkyne with two C—H bonds replaced by points of attachment of the alkynylene group to the remainder of the polymer. Examples are divalent straight hydrocarbon groups consisting of alkynyl groups, such as —C≡C— and —C≡C—CH₂—. The (C_{x-y})alkylene groups include (C₂₋₆)alkynylene and C₂₋₃)alkynylene.

The term “alkoxy” refers to an alkyl group having an oxygen attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxy.

The term “cycloalkyl”, employed alone or in combination with other terms, refers to a non-aromatic, saturated, monocyclic, bicyclic or polycyclic hydrocarbon ring system, including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally substituted by oxo or sulfido. Cycloalkyl groups also include cycloalkylidenes. Example cycloalkyl groups include cyclopropyl, cyclobutyl, cyclopentyl, cyclohexyl, cyclopentenyl, cyclohexenyl, cyclohexadienyl, norbornyl, norpinyl, bicyclo[2.1.1]hexanyl, bicyclo[1.1.1]pentanyl and the like. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like, e.g., indanyl or tetrahydronaphthyl. A cycloalkyl group containing a fused aromatic ring can be attached through any ring-forming atom including a ring-forming atom of the fused aromatic ring.

The term “heterocycloalkyl”, employed alone or in combination with other terms, refers to non-aromatic ring or ring system, which may optionally contain one or more alkenylene groups as part of the ring structure, which has at least one heteroatom ring member independently selected from nitrogen, sulfur, oxygen and phosphorus, and which has 4-10 ring members, 4-7 ring members or 4-6 ring members. Included in heterocycloalkyl are monocyclic 4-, 5-, 6- and 7-membered heterocycloalkyl groups. Heterocycloalkyl groups can include mono- or bicyclic (e.g., having two fused or bridged rings) ring systems. In some embodiments, the heterocycloalkyl group is a monocyclic group having 1, 2 or 3 heteroatoms independently selected from nitrogen, sulfur and oxygen. Examples of heterocycloalkyl groups include azetidine, pyrrolidine, piperidine, piperazine, morpholine, thiomorpholine, pyran, azepane, tetrahydropyran, tetrahydrofuran, dihydropyran, dihydrofuran and the like. Ring-forming carbon atoms and heteroatoms of a heterocycloalkyl group can be optionally substituted by oxo or sulfido (e.g., C(═O), S(═O), C(S) or S(═O)₂, etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include 1, 2, 3, 4-tetrahydroquinoline, dihydrobenzofuran, azetidine, azepane, diazepan (e.g., 1,4-diazepan), pyrrolidine, piperidine, piperazine, morpholine, thiomorpholine, pyran, tetrahydrofuran and di- and tetra-hydropyran.

As used herein, “halo” or “halogen” refers to —F, —Cl, —Br and —I.

As used herein, “aryl,” employed alone or in combination with other terms, refers to an aromatic hydrocarbon group. The aryl group may be composed of, e.g., monocyclic or bicyclic rings and may contain, e.g., from 6 to 12 carbons in the ring, such as phenyl, biphenyl and naphthyl. The term “(C_{x-y})aryl” (wherein x and y are integers) denotes an aryl group containing from x to y ring carbon atoms. Examples of a (C₆₋₁₄)aryl group include, but are not limited to, phenyl, α-naphthyl, β-naphthyl, biphenyl, anthryl, tetrahydronaphthyl, fluorenyl, indanyl, biphenylenyl and acenanaphthyl. Examples of a C₆₋₁₀ aryl group include, but are not limited to, phenyl, α-naphthyl, β-naphthyl, biphenyl and tetrahydronaphthyl.

An aryl group can be unsubstituted or substituted. A substituted aryl group can be substituted with one or more groups, e.g., 1, 2 or 3 groups, including: C₁₋₆)alkyl, (C₂₋₆)alkenyl, (C₂₋₆)alkynyl, halogen, C₁₋₆)haloalkyl, —CN, —NO₂, —C(═O)R, —C(═O)OR, —C(═O)NR₂, —C(═NR)NR₂, —NR₂, —NRC(═O)R, —NRC(═O)O(C₁₋₆)alkyl, —NRC(═O)NR₂, —NRC(═NR)NR₂, —NRSO₂R, —OR, —O(C₁₋₆)haloalkyl, —OC(═O)R, —OC(═O)OC₁₋₆)alkyl, —OC(═O)NR₂, —SR, —S(O)R, —SO₂R, —OSO₂(C₁₋₆)alkyl, —SO₂NR₂, —(C₁₋₆)alkylene-CN, —(C₁₋₆)alkylene-C(═O)OR, —(C₁₋₆)alkylene-C(═O)NR₂, —(C₁₋₆)alkylene—OR, —(C₁₋₆)alkylene—OC(═O)R, —(C₁₋₆)alkylene—NR₂, —(C₁₋₆)alkylene—NRC(═O)R, —NR(C₁₋₆)alkylene-C(═O)OR, —NR(C₁₋₆)alkylene-C(═O)NR₂, —NR(C₂₋₆)alkylene—OR, —NR(C₂₋₆)alkylene—OC(═O)R, —NR(C₂₋₆)alkylene—NR₂, —NR(C₂₋₆)alkylene—NRC(═O)R, —O(C₁₋₆)alkylene-C(═O)OR, —O(C₁₋₆)alkylene-C(═O)NR₂, —O(C₂₋₆)alkylene—OR, —O(C₂₋₆)alkylene—OC(═O)R, —O(C₂₋₆)alkylene—NR₂ and —O(C₂₋₆)alkylene—NRC(═O)R, wherein each R group is hydrogen or (C₁₋₆ alkyl).

The terms “heteroaryl” or “heteroaromatic” as used herein refer to an aromatic ring system having at least one heteroatom in at least one ring, and from 2 to 9 carbon atoms in the ring system. The heteroaryl group has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaryls include furyl, thienyl, pyridyl, oxazolyl, pyrrolyl, indolyl, quinolinyl or isoquinolinyl, and the like. The heteroatoms of the heteroaryl ring system can include heteroatoms selected from one or more of nitrogen, oxygen and sulfur.

Examples of heteroaryl groups include: pyridyl, pyrazinyl, pyrimidinyl, particularly 2- and 4-pyrimidinyl, pyridazinyl, thienyl, furyl, pyrrolyl, particularly 2-pyrrolyl, imidazolyl, thiazolyl, oxazolyl, pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heteroaryls include: indolyl, particularly 3-, 4-, 5-, 6- and 7-indolyl, indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1, 5-naphthyridinyl, 1, 8-naphthyridinyl, 1,4-benzodioxanyl, coumarin, dihydrocoumarin, benzofuryl, particularly 3-, 4-, 5-, 6- and 7-benzofuryl, 2, 3-dihydrobenzofuryl, 1,2-benzisoxazolyl, benzothienyl, particularly 3-, 4-, 5-, 6- and 7-benzothienyl, benzoxazolyl, benzthiazolyl, purinyl, benzimidazolyl, and benztriazolyl.

A heteroaryl group can be unsubstituted or substituted. A substituted heteroaryl group can be substituted with one or more groups, e.g., 1, 2 or 3 groups, including: (C₁₋₆)alkyl, (C₂₋₆)alkenyl, (C₂₋₆)alkynyl, halogen, (C₁₋₆)haloalkyl, —CN, —NO₂, —C(═O)R, —C(═O)OR, —C(═O)NR₂, —C(═NR)NR₂, —NR₂, —NRC(═O)R, —NRC(═O)O(C₁₋₆)alkyl, —NRC(═O)NR₂, —NRC(═NR)NR₂, —NRSO₂R, —OR, —O(C₁₋₆)haloalkyl, —OC(═O)R, —OC(═O)O(C₁₋₆)alkyl, —OC(═O)NR₂, —SR, —S(O)R, —SO₂R, —OSO₂(C₁₋₆)alkyl, —SO₂NR₂, —(C₁₋₆)alkylene—CN, —(C₁₋₆)alkylene-C(═O)OR, —(C₁₋₆)alkylene-C(═O)NR₂, —(C₁₋₆)alkylene—OR, —(C₁₋₆)alkylene—OC(═O)R, —(C₁₋₆)alkylene—NR₂, —(C₁₋₆)alkylene—NRC(═O)R, —NR(C₁₋₆)alkylene-C(═O)OR, —NR(C₁₋₆)alkylene-C(═O)NR₂, —NR(C₂₋₆)alkylene—OR, —NR(C₂₋₆)alkylene—OC(═O)R, —NR(C₂₋₆)alkylene—NR₂, —NR(C₂₋₆)alkylene—NRC(═O)R, —O(C₁₋₆)alkylene-C(═O)OR, —O(C₁₋₆)alkylene-C(═O)NR₂, —O(C₂₋₆)alkylene—OR, —O(C₂₋₆)alkylene—OC(═O)R, —O(C₂₋₆)alkylene—NR₂ and —O(C₂₋₆)alkylene—NRC(═O)R, wherein each R group is hydrogen or (C₁₋₆ alkyl).

The aforementioned listing of heteroaryl moieties is intended to be representative and not limiting.

The term “nanoparticle” as used herein refers to a particle having a size from about 1 nm to about 1000 nm.

The term “nanoparticle size” as used herein refers to the median size in a distribution of nanoparticles. The median size is determined from the average linear dimension of individual nanoparticles, for example, the diameter of a spherical nanoparticle. Size may be determined by any number of methods in the art, including dynamic light scattering (DLS) and transmission electron microscopy (TEM) techniques. In some embodiments, the nanoparticle has a size from about 5 to about 1000 nm, 5 to about 500 nm, from about 5 to about 200 nm, and/or from about 5 to about 100 nm.

The term “nanofiber” refers to a nanoparticle having a fiber-like presentation.

The term “nanosphere” refers to a nanoparticle having a spherical shape. Accordingly, nanospheres exhibit a visibly uniform radius that define their boundaries. The size of a nanosphere refers to its average diameter.

The term “nanorod” refers to a nanoparticle having a rod-like shape defined by a length and width.

The term “nano round column” refers to a nanoparticle shape between a nanosphere and a nanorod. The structure resembles an elongated nanosphere or a short nanorod.

The term “nanoring” refers to a nanoparticle having a ring-like shape.

The term “bell-rod” refers to a nanoparticle having a shape like a nanorod attached to one or more nanospheres.

The term “protecting group” refers to a chemical functional group that can be used to derivatize a reactive functional group present in a molecule to prevent undesired reactions from occurring under particular sets of reaction conditions but which is capable of being introduced and removed selectively under known reaction conditions. The chemistry and use of functional groups is familiar to one skilled in the art. Discussion of protecting groups can be found, e.g., in Protecting Group Chemistry, 1^{st} Ed., Oxford University Press, 2000; March's Advanced Organic chemistry: Reactions, Mechanisms, and Structure, 5^{th} Ed., Wiley Interscience Publication, 2001; Peturssion, S. et al., “Protecting Groups in Carbohydrate Chemistry,” J. Chem. Educ., 1997, 74(11), 1297, Wuts et al., Protective Groups in Organic Synthesis, 4^{th} Ed., Wiley Interscience (2007).

The term “substituted” means that an atom or group of atoms formally replaces hydrogen as a “substituent” attached to another group. The term “substituted”, unless otherwise indicated, refers to any level of substitution, namely mono-, di-, tri-, tetra- or penta-substitution, where such substitution is permitted. The substituents are independently selected, and substitution may be at any chemically accessible position. When groups are described herein as being substituted, the substituents can include, but are not limited to, (C₁₋₆)alkyl, (C₂₋₆)alkenyl, (C₂₋₆)alkynyl, halogen, (C₁₋₆)haloalkyl, —CN, —NO₂, —C(═O)R, —OC(═O)Ar, —C(═O)OR, —C(═O)NR₂, —C(═NR)NR₂, —OR, —Ar, —OAr, —((C₁₋₆)alkylene)Ar, —O((C₁₋₆)alkylene)Ar, —OC(═O)(C₁₋₆)alkyl, —OC(═O)O(C₁₋₆)alkyl, —OC(═O)NR₂, —NR₂, —NRAr, —NR((C₁₋₆)alkylene)Ar, —NRC(═O)R, —NRC(═O)Ar, —NRC(═O)O(C₁₋₆)alkyl, —NRC(═O)NR₂, —NRSO₂R, —SR, —S(O)R, —SO₂R, —OSO₂(C₁₋₆)alkyl, —SO₂NR₂, (C₁₋₈)perfluoroalkyl, —(C₂₋₆)alkylene—OR, —O(C₂₋₆)alkylene-N((C₁₋₆)alkyl)₂, —P(═O)(OR)₂, —OP(═O)(OR)₂, wherein each R group is hydrogen or (C₁₋₆alkyl), e.g., methyl and wherein each Ar is independently unsubstituted aryl or heteroaryl or aryl or heteroaryl substituted with one or more of —(C₁₋₆)alkyl, (C₂₋₆)alkenyl, (C₂₋₆)alkynyl, halogen, (C₁₋₆)haloalkyl, —CN, —NO₂, —C(═O)R, —C(═O)OR, —C(═O)NR₂, —C(═NR)NR₂, —OR, —OC(═O)(C₁₋₆)alkyl, —OC(═O)O(C₁₋₆)alkyl, —OC(═O)NR₂, —NR₂, —NRC(═O)R, —NRC(═O)O(C₁₋₆)alkyl, —NRC(═O)NR₂, —NRSO₂R, —SR, —S(O)R, —SO₂R, —OSO₂(C₁₋₆)alkyl, —SO₂NR₂, (C₁₋₈)perfluoroalkyl, —(C₂₋₆)alkylene—OR, —O(C₂₋₆)alkylene-N((C₁₋₆)alkyl)₂, —P(═O)(OR)₂, —OP(═O)(OR)₂ wherein each R group is hydrogen or C₁₋₆ alkyl).

The term “salt” includes any ionic form of a polymer and one or more counterionic species (cations and/or anions). Salts also include zwitterionic polymers (i.e., a molecule containing one more cationic and anionic species, e.g., zwitterionic amino acids). Counter ions present in a salt can include any cationic, anionic, or zwitterionic species. Exemplary anions include, but are not limited to, chloride, bromide, iodide, nitrate, sulfate, bisulfate, sulfite, bisulfite, phosphate, acid phosphate, perchlorate, chlorate, chlorite, hypochlorite, periodate, iodate, iodite, hypoiodite, carbonate, bicarbonate, isonicotinate, acetate, trichloroacetate, trifluoroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, trifluoromethansulfonate, ethanesulfonate, benzenesulfonate, p-toluenesulfonate, p-trifluoromethylbenzenesulfonate, hydroxide, aluminates and borates. Exemplary cations include, but are not limited to, monovalent alkali metal cations, such as lithium, sodium, potassium and cesium, and divalent alkaline earth metals, such as beryllium, magnesium, calcium, strontium and barium. Also included are transition metal cations, such as gold, silver, copper and zinc, as well as nonmetal cations, such as ammonium salts. Representative organic amines useful for the formation of base addition salts include ethylamine, diethylamine, ethylenediamine, ethanolamine, diethanolamine, piperazine, and the like.

For example, a polymer of the invention having a structure according to Formula (I), where R¹=H, may exist in either the free acid form or in a corresponding salt form with a cation, such as K⁻ or ethylammonium salt.

References to a polymer described and disclosed herein are considered to include the free acid, the free base, and all addition salts and complexes of the polymer. The polymers may also form inner salts or zwitterions when a free carboxy and a basic amino group are present concurrently. The term “pharmaceutically acceptable salt” refers to salts which possess toxicity profiles within a range that affords utility in pharmaceutical applications. Pharmaceutically unacceptable salts may nonetheless possess properties such as high crystallinity, which may render them useful, e.g., in processes of synthesis, purification or formulation of polymers described herein. In general the useful properties of the polymers described herein do not depend on whether the polymer is or is not in a salt form, so unless clearly indicated otherwise (such as specifying that the polymer should be in “free base” or “free acid” form), reference in the specification to a polymer should be understood as including salt forms of the polymer, whether or not this is explicitly stated. Preparation and selection of suitable salt forms is described in Stahl et al., Handbook of Pharmaceutical Salts: Properties, Selection, and Use, Wiley-VCH 2002.

When in the solid state, the polymers described herein and salts thereof may occur in various forms and may, e.g., take the form of solvates, including hydrates. In general, the useful properties of the polymers described herein do not depend on whether the polymer or salt thereof is or is in a particular solid state form, such as a polymorph or solvate, so unless clearly indicated otherwise reference in the specification to polymers and salts should be understood as encompassing any solid state form of the polymer, whether or not this is explicitly stated.

Polymers provided herein can also include all isotopes of atoms occurring in the intermediates or final polymers. Isotopes include those atoms having the same atomic number but different mass numbers. For example, isotopes of hydrogen include tritium and deuterium.

The phrase “pharmaceutically acceptable” is employed herein to refer to those polymers, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The following abbreviations may be used herein: AcOH (acetic acid); Ag (silver); AgNO₃ (silver nitrate); aq. (aqueous); atm. (atmosphere(s)); Da (dalton(s)); dd (doublet of doublets); DCM (dichloromethane); DIPEA (N,N-diisopropylethylamine); Dil (1,1′-dioctadecyl-3,3,3′, 3′-tetramethylindocarbocyanine perchlorate (or another salt thereof)); DiR (1,1′-dioctadecyl-3,3,3′, 3′-tetramethylindotricarbocyanine Iodide) DMF (N,N-dimethylformamide); DLS (dynamic light scattering); DMEM (Dulbecco's Modified Eagle Medium); DMSO (dimethylsulfoxide); DSC (differential scaning calorimetry); DSPE-PEG 1,2-distearoyl-sn-glycero-3-phosphoethanolamine-poly(ethylene glycol); DTT (dithiothreitol); Dtxl (docetaxel); Et (ethyl); Et3N or TEA (triethylamine); EtOAc (ethyl acetate); EtOH (ethanol); FBS (fetal bovine serum); FeCl₂ (iron (ii) chloride); g (gram(s)); GPC (gel permeation chromatography); GSH (glutathione); h (hour(s)); HPLC (high performance liquid chromatography); Li (lithium); M (molar); Me (methyl); MeCN (acetonitrile); MeOH (methanol); mg (milligram(s)); min. (minute(s)); mL (milliliter(s)); mmol (millimole(s)); mV (millivolt(s)); MRI (magnetic resonance imaging); Mn or MW (molecular weight); N (normal); nm (nanometer); nM (nanomolar); NMR (nuclear magnetic resonance spectroscopy); NP (nanoparticle); NPs (nanoparticles); nPn (n-pentyl); nPr (n-propyl); PBS (phosphate-buffered saline); PDSA (poly(disulfide amide)); PEG (polyethylene glycol); PLGA (poly lactic (co-glycolic) acid); PVA (polyvinyl alcohol); rpm (revolutions per minute); s (second(s)); t-Bu (tent-butyl); TEA (triethylamine); TCEP (tris(2-carboxyethyl)phosphine; TEM (transmission electron microscopy); T_{g} (glass transition temperature); TFA (trifluoroacetic acid); THF (tetrahydrofuran); μg (microgram(s)); μL (microliter(s)); μm (micromolar); wt (weight); wt % (weight percent).

### II. Novel Polymers

### A. Polymers

This disclosure provides a polymer comprising at least one repeating unit according to Formula (I):

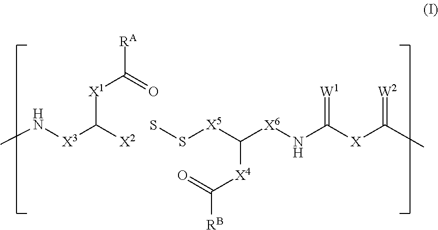

wherein

X¹is a bond or C₁₋₁₀₀ alkylene;

X² is C₁₋₁₀₀ alkylene;

X³ is a bond or C₁₋₁₀₀ alkylene;

X⁴ is a bond or C₁₋₁₀₀ alkylene;

X⁵ is C₁₋₁₀₀ alkylene;

X⁶ is a bond or C₁₋₁₀₀ alkylene;

R^{A} is OR¹ or NR¹R⁴;

R^{B} is OR² or NR²R⁴;

R¹ is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₁₀₀ alkyl or C(═O)R⁶;

each R⁴ is independently H or C₁₋₁₀₀ alkyl;

each R⁵ is independently H or C₁₋₁₀₀ alkyl;

each R⁶ is independently H or C₁₋₁₀₀ alkyl;

W¹ is O, S, or NH;

W² is O, S, or NH;

X is C₁₋₁₀₀ alkylene, C₂₋₁₀₀ alkenylene, or C₂₋₁₀₀ alkynylene;

provided that when W¹ and W² are both O, then X is C₃₋₁₀₀ alkylene, C₂₋₁₀₀ alkenylene, or C₂₋₁₀₀ alkynylene; and

each m is 0, 1 or 2.

In some embodiments, X¹is a bond or C₁₋₄ alkylene.

In some embodiments, X² is C₁₋₄ alkylene.

In some embodiments, X³ is a bond or C₁₋₄ alkylene.

In some embodiments, X⁴ is a bond or C₁₋₄ alkylene.

In some embodiments, X⁵ is C₁₋₄ alkylene.

In some embodiments, X⁶ is a bond or C₁₋₄ alkylene.

In some embodiments, R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl.

In some embodiments, R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl.

In some embodiments, each R³ is independently H, C₁₋₆ alkyl or C(═O)R⁶.

In some embodiments, each R⁴ is independently H or C₁₋₆ alkyl.

In some embodiments, each R⁵ is independently H or C₁₋₆ alkyl.

In some embodiments, each R⁶ is independently H or C₁₋₆ alkyl.

In some embodiments, X is C₂₋₂₀ alkylene, C₂₋₂₀ alkenylene, or C₂₋₂₀ alkynylene.

In some embodiments,

X¹is a bond or C₁₋₄ alkylene;

X² is C₁₋₄ alkylene;

X³ is a bond or C₁₋₄ alkylene;

X⁴ is a bond or C₁₋₄ alkylene;

X⁵ is Cl-4 alkylene;

X⁶ is a bond or C₁₋₄ alkylene;

R^{A} is OR¹ or NR¹R⁴;

R^{B} is OR² or NR²R⁴;

R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₆ alkyl or C(═O)R⁶;

each R⁴ is independently H or C₁₋₆ alkyl;

each R⁵ is independently H or C₁₋₆ alkyl;

each R⁶ is independently H or C₁₋₆ alkyl;

W¹ is O, S, or NH;

W² is O, S, or NH;

X is C₂₋₂₀ alkylene, C₂₋₂₀ alkenylene, or C₂₋₂₀ alkynylene; and

each m is 0, 1 or 2.

In some embodiments, when W¹ is O and W² is O, X is C₃₋₂₀ alkylene, C₂₋₂₀ alkenylene, or C₂₋₂₀ alkynylene. For example, X can be C₃₋₂₀ alkylene.

In some embodiments, when W¹ is O and W² is O, X is C₄₋₂₀ alkylene, C₂₋₂₀ alkenylene, or C₂₋₂₀ alkynylene. For example, X can be C₄₋₂₀ alkylene.

In some embodiments, X¹is a bond.

In some embodiments, X² is C₁₋₄ alkylene. For example, X² can be CH₂.

In some embodiments, X³ is a bond.

In some embodiments, X⁴ is a bond.

In some embodiments, X⁵ is C₁₋₄ alkylene. For example, X⁵ can be CH₂.

In some embodiments, X⁶ is a bond.

In some embodiments, R^{A} is OR'.

In some embodiments, R^{B} is OR².

In some embodiments, W¹ is O.

In some embodiments, W² is O.

In some embodiments, a polymer of Formula (I) has at least one repeating unit with a structure according to Formula (Ia):

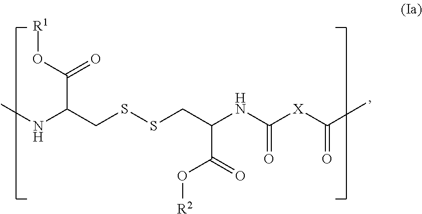

wherein:

R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;

each R³ is independently H, C₁₋₆ alkyl or C(═O)R⁶;

each R⁴ is independently H or C₁₋₆ alkyl;

each R⁵ is independently H or C₁₋₆ alkyl;

each R⁶ is independently H or C₁₋₆ alkyl;

X is C₃₋₂₀ alkylene, alkenylene, or alkynylene; and

each m is 0, 1 or 2.

In some embodiments, R¹ is H, C₁₋₂₀ alkyl, C₂₋₂₀ alkenyl, C₂₋₂₀ alkynyl, C₃₋₁₀ cycloalkyl, or C₆₋₁₀ aryl. For example, R¹ can be H. In some embodiments, R¹ is C₁₋₂₀ alkyl. In some embodiments, R¹ is C₁₋₆ alkyl. For example, R¹ can be CH₃. In some embodiments, R¹ is CH₂CH₃.

In some embodiments, R² is H, C₁₋₂₀ alkyl, C₂₋₂₀ alkenyl, C₂₋₂₀ alkynyl, C₃₋₁₀ cycloalkyl, or C₆₋₁₀ aryl. For example, R² can be H. In some embodiments, R² is C₁₋₂₀ alkyl. In some embodiments, R² is C₁₋₆ alkyl. For example, R² can be CH₃. In some embodiments, R² is CH₂CH₃.

In some embodiments, R³ is C₁₋₆ alkyl. For example, R³ can be CH₃. In some embodiments, R³ is H.

In some embodiments, R⁴ is C₁₋₆ alkyl. For example, R⁴ can be CH₃.

In some embodiments, R⁵ is C₁₋₆ alkyl. For example, R⁵ can be CH₃.

In some embodiments, R⁶ is C₁₋₆ alkyl. For example, R⁶ can be CH₃.

In some embodiments, m is 0. In some embodiments, m is 2.

The length and nature of the X group can be used to modulate the hydrophobicity of a polymer of Formula (I) and/or Formula (Ia). X groups may include alkylenes, including C₃₋₂₀ alkylenes (e.g., (CH₂)₃₋₂₀) and C₄₋₁₀ alkylenes (e.g., CH₂)₄₋₁₀). Specific alkylene groups include C₄ alkylenes (e.g., (CH₂)₄), C₅ alkylenes (e.g., (CH₂)₅), C₆ alkylenes (e.g., (CH₂)₆), C₇ alkylenes (e.g., (CH₂)₇), C₅ alkylenes (e.g., (CH₂)₈), C₉ alkylenes (e.g., (CH₂)₉), C₁₀ alkylenes (e.g., (CH₂)₁₀), C₁₁ alkylenes (e.g., (CH₂)₁₁), and C₁₂ alkylenes (e.g., (CH₂)₁₂).

Examples of a repeating unit in a polymer of Formula (I) and/or Formula (Ia) where X is (CH₂)₄ include:

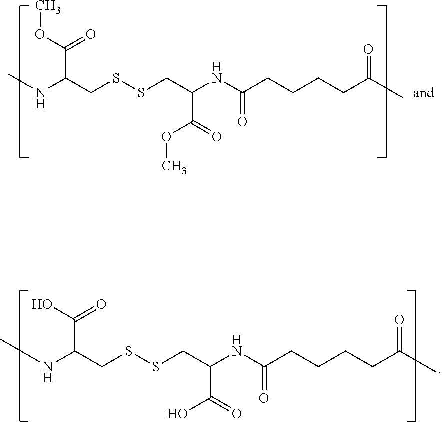

Examples of a repeating unit in a polymer of Formula (I) and/or Formula (Ia) where X is (CH₂)₆ include:

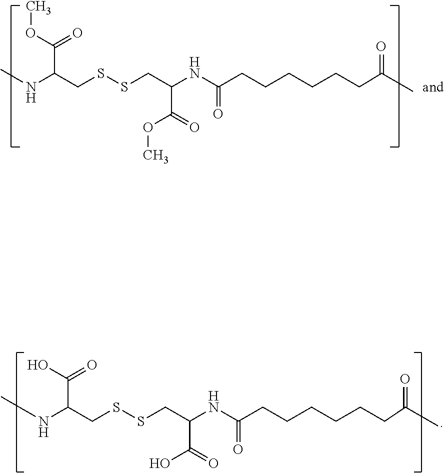

Examples of a repeating unit in a polymer of Formula (I) and/or Formula (Ia) where X is (CH₂)₈ include:

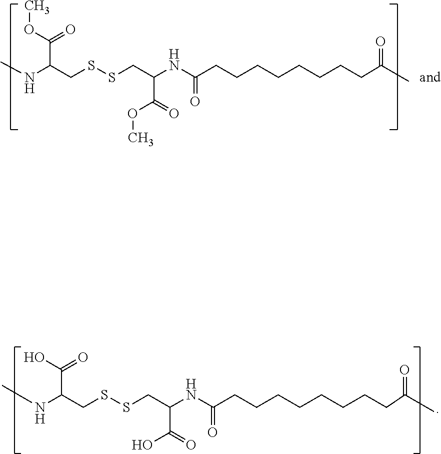

Examples of a repeating unit in a polymer of Formula (I) and/or Formula (Ia) where X is (CH₂)₁₀ include:

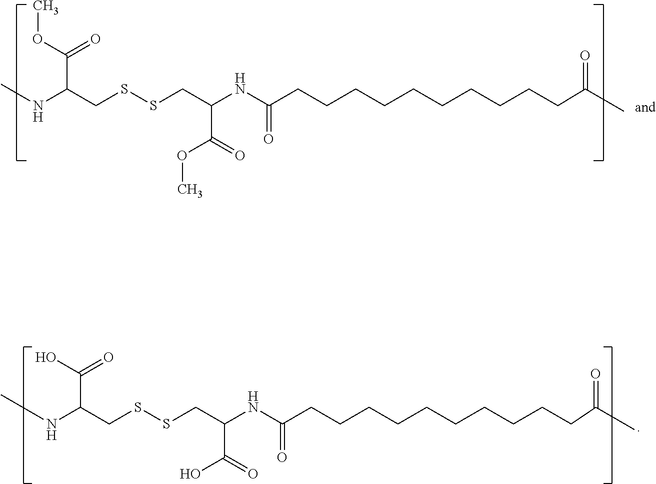

Also provided is a polymer comprising at least one repeating unit according to Formula (II):

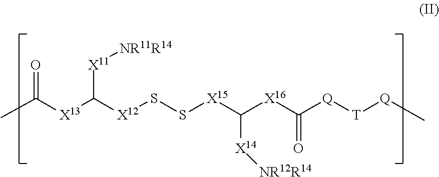

wherein:

X¹¹ is a bond or C₁₋₁₀₀ alkylene;

X¹² is C₁₋₁₀₀ alkylene;

X¹³ is a bond or C₁₋₁₀₀ alkylene;

X¹⁴ is a bond or C₁₋₁₀₀ alkylene;

X¹⁵ is C₁₋₁₀₀ alkylene;

X¹⁶ is a bond or C₁₋₁₀₀ alkylene;

R¹¹ is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹¹ is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)R¹⁴, and C₆₋₁₀ aryl;

R¹² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹² is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)R¹⁴, and C₆₋₁₀ aryl;

each R¹³ is independently H, C₁₋₁₀₀ alkyl or C(═O)R¹⁶;

each R¹⁴ is independently H or C₁₋₁₀₀ alkyl;

each R¹⁵ is independently H or C₁₋₁₀₀ alkyl;

each R¹⁶ is independently H or C₁₋₁₀₀ alkyl;

each Q is independently O or NR¹⁷;

each R¹⁷ is H or C₁₋₁₀₀ alkyl;

T is C₂₋₁₀₀ alkylene, C₄₋₁₀₀ alkenylene, or C₄₋₁₀₀ alkynylene; and

each n is 0, 1 or 2.

In some embodiments, X¹¹ is a bond or C₁₋₄ alkylene.

In some embodiments, X¹² is C₁₋₄ alkylene.

In some embodiments, X¹³ is a bond or C₁₋₄ alkylene.

In some embodiments, X¹⁴ is a bond or C₁₋₄ alkylene.

In some embodiments, X¹⁵ is C₁₋₄ alkylene.

In some embodiments, X¹⁶ is a bond or C₁₋₄ alkylene.

In some embodiments, R¹¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(═O)R⁴, —(C═O)OR⁴, —(═O)NR⁴R⁵, —S(O)ₘR⁴, and C₆₋₁₀ aryl.

In some embodiments, R¹² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl.

In some embodiments, each R¹³ is independently H, C₁₋₆ alkyl or C(═O)R⁶.

In some embodiments, each R¹⁴ is independently H or C₁₋₆ alkyl.

In some embodiments, each R¹⁵ is independently H or C₁₋₆ alkyl.

In some embodiments, each R¹⁶ is independently H or C₁₋₆ alkyl.

In some embodiments, T is C₂₋₂₀ alkylene, C₂₋₂₀ alkenylene, or C₂₋₂₀ alkynylene.

In some embodiments,

X¹¹ is a bond or C₁₋₄ alkylene;

X¹² is C₁₋₄ alkylene;

X¹³ is a bond or C₁₋₄ alkylene;

X¹⁴ is a bond or C₁₋₄ alkylene;

X¹⁵ is C₁₋₄ alkylene;

X¹⁶ is a bond or C₁₋₄ alkylene;

R¹¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹¹ is optionally substituted with 1, 2, or 3 substituents independently selected from the group consisting of: halo, —CN, OR³¹³, NR¹³R¹⁴, —(C═O)R¹⁴, —(C═O)OR¹⁴, —(C═O)NR¹⁴R¹⁵, —S(O)R¹⁴, and C₆₋₁₀ aryl;

R¹² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹² is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;

each R¹³ is independently H, C₁₋₆ alkyl or C(═O)R¹⁶;

each R¹⁴ is independently H or C₁₋₆ alkyl;

each R¹⁵ is independently H or C₁₋₆ alkyl;

each R¹⁶ is independently H or C₁₋₆ alkyl;

each Q is independently O or NR¹⁷;

each R¹⁷ is independently H or C₁₋₆ alkyl;

T is C₂₋₂₀ alkylene, C₄₋₂₀ alkenylene, or C₄₋₂₀ alkynylene; and

each n is 0, 1 or 2.

In some embodiments, X¹¹ is a bond.

In some embodiments, X¹² is C₁₋₄ alkylene. For example, X¹² can be CH₂.

In some embodiments, X¹³ is a bond.

In some embodiments, X¹⁴ is a bond.

In some embodiments, X¹⁵ is C₁₋₄ alkylene. For example, X¹⁵ can be CH₂.

In some embodiments, X¹⁶ is a bond.

In some embodiments, R¹¹ is H, C₁₋₂₀ alkyl, C₂₋₂₀ alkenyl, C₂₋₂₀ alkynyl, C₃₋₁₀ cycloalkyl, or C₆₋₁₀ aryl. For example, R^{H} can be H. In some embodiments, R¹¹ is C₁₋₂₀ alkyl. In some embodiments, R^{H} is C₁₋₆ alkyl. For example, R¹¹ can be CH₃. In some embodiments, R^{H} is CH₂CH₃.

In some embodiments, R¹² is H, C₁₋₂₀ alkyl, C₂₋₂₀ alkenyl, C₂₋₂₀ alkynyl, C₃₋₁₀ cycloalkyl, or C₆₋₁₀ aryl. For example, R¹² can be H. In some embodiments, R¹² is C₁₋₂₀ alkyl. In some embodiments, R¹² is C₁₋₆ alkyl. For example, R¹² can be CH₃. In some embodiments, R¹² is CH₂CH₃.

In some embodiments, R¹³ is C₁₋₆ alkyl. For example, R¹³ can be CH₃. In some embodiments, R¹³ is H.

In some embodiments, R¹⁴ is C₁₋₆ alkyl. For example, R¹⁴ can be CH₃.

In some embodiments, R¹⁵ is C₁₋₆ alkyl. For example, ^{R′5} can be CH₃.

In some embodiments, R¹⁶ is C₁₋₆ alkyl. For example, R¹⁶ can be CH₃.

In some embodiments, n is 0. In some embodiments, n is 2.

In some embodiments, Q is O.

The length and nature of the T group can be used to modulate the hydrophobicity of a polymer of Formula (II). T groups may include alkylenes, including C₃₋₂₀ alkylenes (e.g., (CH₂)₃₋₂₀) and C₄₋₁₀ alkylenes (e.g., CH₂)₄₋₁₀). Specific alkylene groups include C₄ alkylenes (e.g., (CH₂)₄), C₅ alkylenes (e.g., (CH₂)₅), C₆ alkylenes (e.g., (CH₂)₆), C₇ alkylenes (e.g., (CH₂)₇), C₅ alkylenes (e.g., (CH₂)₈), C₉ alkylenes (e.g., (CH₂)₉), C₁₀ alkylenes (e.g., (CH₂)₁₀), Cil alkylenes (e.g., (CH₂)₁₁), and C₁₂ alkylenes (e.g., (CH₂)₁₂).

Examples of a repeating unit of a polymer of Formula (II) include:

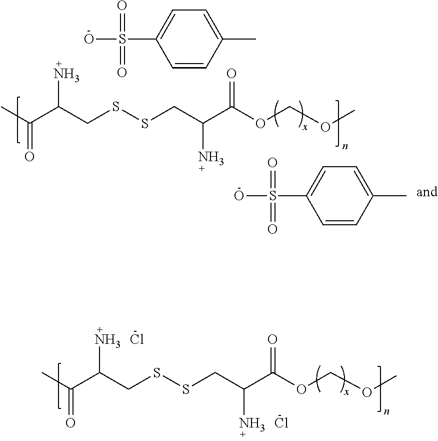

wherein x is an integer from 2 to 100.

In some embodiments, a polymer of Formula (I), Formula (Ia), and/or Formula (II) is a homopolymer comprising only the repeating unit according to the Formula. In some embodiments, a polymer of Formula (I), Formula (Ia), and/or Formula (II) is a copolymer comprising at least one repeating unit according to the Formula. For example, a polymer of Formula (I), Formula (Ia), and/or Formula (II) can be a copolymer comprising at least one repeating unit according to the Formula and PLGA (poly lactic (co-glycolic) acid).

In some embodiments, a polymer of Formula (I), Formula (Ia), and/or Formula (II) is a linear polymer. In some embodiments, a polymer of Formula (I), Formula (Ia), and/or Formula (II) is a branched polymer. In some embodiments, a polymer of Formula (I), Formula (Ia), and/or Formula (II) is a cross-linked polymer.

Terminal end groups for a polymer of Formula (I), Formula (Ia), and/or Formula (II) are known in the art, and can be any protecting groups, drugs, dyes, imaging reagents, targeting ligands, biological molecules which may terminate the polymerization process. For example, an N-terminal end group can be H, alkyl, alkenyl, alkynyl, cycloalkyl, aryl, heteroaryl, heterocyclyl, amide, sulfonamide, sulfamate, sulfinamide, or carbamate. A C-terminal end group can be carboxylic acid, ester, amide, or ketone of alkyl, alkenyl, alkynyl, cycloalkyl, aryl, heteroaryl, or heterocyclyl. For example, a drug molecule having an alcohol function, such as docetaxel, may be used as a C-terminal end group by attachment as an ester.

The molecular weight of a polymer of Formula (I), Formula (Ia), and/or Formula (II) can be determined by any means known in the art. In some embodiments, the number average molecular weight (Mₙ) of a polymer of Formula (I), Formula (Ia), and/or Formula (II) is determined by gel permeation chromatography (GPC). Typically, a polymer of Formula (I), Formula (Ia), and/or Formula (II) has from about 2 to about 100,000 repeating units. In some embodiments, the M. of the polymer is in the range from about 600 to about 10,000,000 daltons, about 600 to about 150,000 daltons, about 600 to about 140,000 daltons, about 600 to about 130,000 daltons, about 600 to about 120,000 daltons, about 600 to about 110,000 daltons, about 600 to about 100,000 daltons, from about 600 to about 90,000 daltons, from about 600 to about 80,000 daltons, from about 600 to about 70,000 daltons, from about 600 to about 60,000 daltons, from about 600 to about 50,000 daltons, from about 600 to about 40,000 daltons, from about 600 to about 30,000 daltons, from about 600 to about 20,000 daltons, from about 600 to about 10,000 daltons, from about 600 to about 9,000 daltons, from about 600 to about 8,000 daltons, from about 600 to about 7,000 daltons, from about 600 to about 6,000 daltons, from about 600 to about 5,000 daltons, from about 600 to about 4,000 daltons, and/or from about 600 to about 3,000 daltons.

The polydispersity of a polymer of Formula (I), Formula (Ia), and/or Formula (II) can be determined by means known in the art. As used herein, the polydispersity or dispersity of a polymer measures the degree of uniformity in size of the polymer. In some embodiments, the polydispersity of a polymer of Formula (I), Formula (Ia), and/or Formula (II) is determined by gel permeation chromatography (GPC).

Without being limited to the following procedures, general schemes for the synthesis of a polymer of Formula (I), Formula (Ia), and/or Formula (II) include a polycondensation method that involves a cysteine monomer and a bis-activated ester or diacid chloride, as shown in the non-limiting example of Scheme 1, where x is the length of the methylene linker (e.g., x=1-100), and n is the number of repeating units (e.g., n=2-100,000).

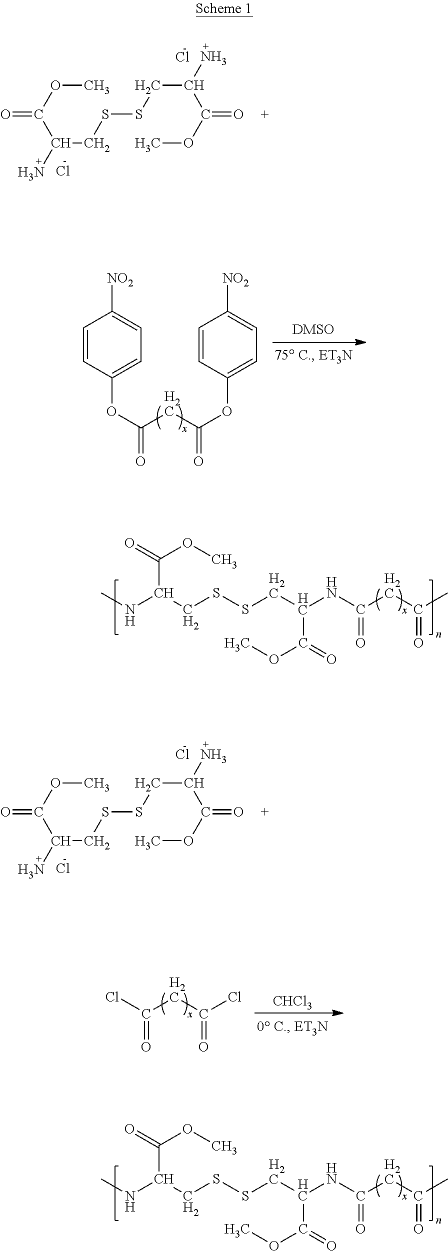

The polymers can also be synthesized by a polycondensation method that forms the cystine —S-S— bond simultaneous with polymerization, as illustrated in Scheme 2, where x is the length of the methylene linker (e.g., x=1-100), and n is the number of repeating units (e.g., n=2-100,000).

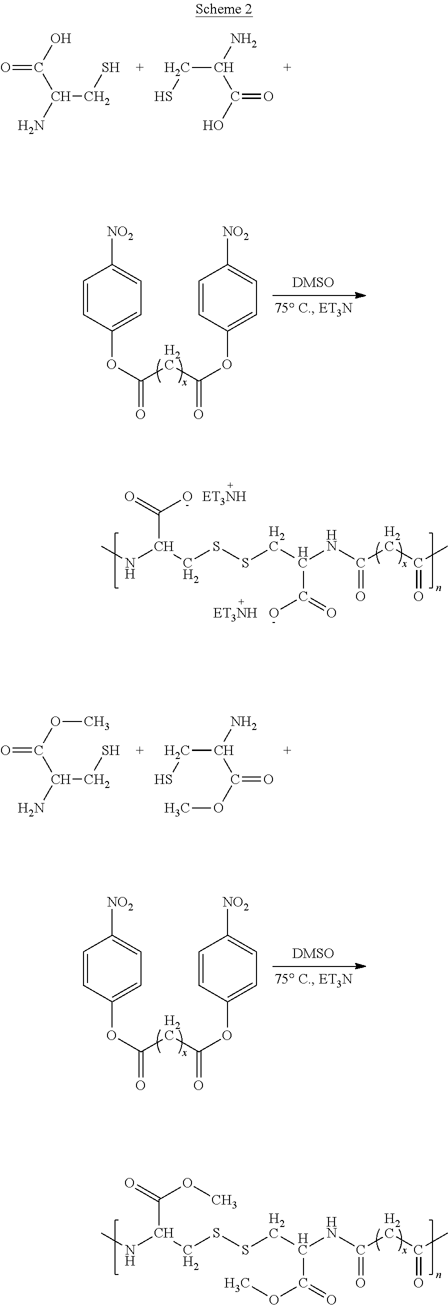

### B. Nanostructures

Also provided in this disclosure is a nanoparticle comprising a polymer as described herein. A nanoparticle may have a variety of shapes, depending on, e.g., the concentration of the polymer solution used, temperature, time, polymer structure, and the nature and concentration of any coprecipitation ingredient, such as a drug molecule. For example, the nanoparticle may appear as a nanosphere, a nanorod, a nanofiber, a bell-rod, a nano round column, or a nanoring. The nanoparticles may be provided in primarily one shape, e.g., as a nanosphere, or as a mixture, e.g., a mixture of nanospheres and nanorods. In addition, the nanoparticles may be further organized in more complex tertiary structures such as sheets of nanofibers. In some embodiments, the shape does not change upon vortexing. In some embodiments, the shape changes upon vortexing.

The size of the nanoparticles are from about 1 nm to about 1000 nm. In some embodiments, the size is in the range from about 5 nm to about 1000 nm, from about 5 nm to about 500 nm, from about 5 nm to about 400 nm, from about 5 nm to about 300 nm, from about 5 nm to about 200 nm, from about 5 nm to about 100 nm, from about 20 nm to about 200 nm, from about 40 nm to about 200 nm, from about 60 nm to about 200 nm, from about 20 nm to about 180 nm, from about 40 nm to about 180 nm, from about 60 nm to about 180 nm, from about 20 nm to about 160 nm, from about 40 nm to about 160 nm, from about 60 nm to about 160 nm, and/or from about 75 nm to about 150 nm.

In some embodiments, the nanoparticles present within a population, e.g., in a composition, can have substantially the same shape and/or size (i.e., they are “monodisperse”). For example, the particles can have a distribution such that no more than about 5% or about 10% of the nanoparticles have a diameter greater than about 10% greater than the average diameter of the particles, and in some cases, such that no more than about 8%, about 5%, about 3%, about 1%, about 0.3%, about 0.1%, about 0.03%, or about 0.01% have a diameter greater than about 10% greater than the average diameter of the nanoparticles.

In some embodiments, the diameter of no more than 25% of the nanoparticles varies from the mean nanoparticle diameter by more than 150%, 100%, 75%, 50%, 25%, 20%, 10%, or 5% of the mean nanoparticle diameter. It is often desirable to produce a population of nanoparticles that is relatively uniform in terms of size, shape, and/or composition so that most of the nanoparticles have similar properties. For example, at least 80%, at least 90%, or at least 95% of the nanoparticles produced using the methods described herein can have a diameter or greatest dimension that falls within 5%, 10%, or 20% of the average diameter or greatest dimension. In some embodiments, a population of nanoparticles can be heterogeneous with respect to size, shape, and/or composition. In this regard, see, e.g., WO 2007/150030, which is incorporated herein by reference in its entirety.

### Bio Compatibility

The nanoparticles described herein are biodegradable and/or biocompatible, i.e., a nanoparticle containing polymers that do not typically induce an adverse response when inserted or injected into a living subject, for example, without significant inflammation and/or acute rejection of the polymers by the immune system, for instance, via a T-cell response. One test to determine biocompatibility is to expose polymers to cells in vitro, where biocompatible polymers typically do not result in significant cell death at moderate concentrations, e.g., at concentrations of about 50 μg/10⁶ cells. For example, a biocompatible polymer may cause less than about 20% cell death when exposed to cells such as fibroblasts or epithelial cells, even if phagocytosed or otherwise taken-up by such cells. In some embodiments, the nanoparticle has an anticancer effect. In some embodiments, the nanoparticle can result in significant cell death of cancer cells without an adverse response in normal cells.

The polymers present in the nanoparticles can also be biodegradable, i.e., the polymers are able to degrade, chemically and/or biologically, within a physiological environment, such as within the body. Degradation of the polymers can occur at varying rates, depending on the polymers or copolymers used. For example, the half-life of the polymers (the time at which 50% of the polymers are degraded into monomers and/or other nonpolymeric moieties) can be on the order of days, weeks, months, or years, depending on the particular polymers used to make the nanoparticles. The polymers can be biologically degraded, e.g., by enzymatic activity in cleavage of amide bonds present or cellular machinery, in some cases, for example, through exposure to the reductive environment of a cell to degrade the —S-S— bonds. In some cases, the polymers can be broken down into monomers and/or other nonpolymeric moieties that cells can either reuse or dispose of without significant toxic effect on the cells (e.g., polymer can be hydrolyzed to form cysteine).

### Methods of Making the Nanoparticles

The methods of forming the nanoparticles allow for a uniform synthesis, which affords a uniform size and shape of the resulting nanoparticles. One advantage of the invention allows for the simple and rapid synthesis of nanoparticles by a nanoprecipitation method. In one aspect, a method for preparing a nanoparticle of the disclosure comprises (a) dissolving a polymer of Formula (I), Formula (Ia), and/or Formula (II) in a polar aprotic solvent to give a polymer solution; and (b) adding the polymer solution to water to provide the nanoparticle. For example, the polymer solution can be added dropwise to water to facilitate the nanoprecipitation process. In some embodiments, the polar aprotic solvent is DMSO. In some embodiments, the concentration of the polymer in the polymer solution is in the range from about 0.5 to about 100 mg/mL. In some embodiments, the concentration of the polymer in the polymer solution is in the range from about 1 to about 30 mg/mL. In some embodiments, the concentration of the polymer in the polymer solution is in the range from about 5 to about 10 mg/mL. In some embodiments, a second agent, such as a drug molecule or a detectable agent, is also dissolved in the polymer solution, and subsequently is incorporated into the nanoparticle.

In some embodiments, a stabilizer is added to increase the stability and shelf life of the nanoparticles produced by this method. Common stabilizers include Pluronic® F-127, polyvinyl alcohol having a molecular weight of 13,000-23,000 (PVA), Tween® 80, poly(oxyethylene stearate) (MYRJ™), lipid (such as lecithin), and lipid-PEG (such as 1,2-distearoyl-sn-glycero-3-phosphoethanolamine-poly(ethylene glycol) (DSPE-PEG)).

### C. Compositions

Also provided is a composition comprising a polymer of Formula (I), Formula (Ia), and/or Formula (II) and a payload as described herein. The composition may comprise a nanoparticle as described herein, using a method of synthesizing the nanoparticle described above. In some embodiments, the polymer is not covalently attached to the payload. For example, in some embodiments, the polymer encapsulates the payload, such as a contrast agent. In some embodiments, the polymer is covalently attached to the payload. For example, the polymer can be covalently attached to a drug molecule via a linker.

### Payload

The methods and compositions described herein are useful for delivering a payload. In some embodiments, the payload is delivered to a biological target. The payload can be used, e.g., for labeling (e.g., a detectable agent such as a fluorophore), or for therapeutic purposes (e.g., a cytotoxin or other drug molecule).

The proportion of the payload relative to the polymer used in the composition depends on the characteristics of the payload, the properties of the polymer, and the application. In some embodiments, the payload is loaded in the range from about 0.01% by weight to about 100.0% by weight compared with the weight of the polymer. The payload can be in the range from about 1% by weight to about 80% by weight, from about 1% by weight to about 75% by weight, from about 1% by weight to about 70% by weight, from about 1% by weight to about 65% by weight, from about 1% by weight to about 60% by weight, from about 1% by weight to about 55% by weight, from about 1% by weight to about 50% by weight, from about 1% by weight to about 45% by weight, from about 1% by weight to about 40% by weight, from about 1% by weight to about 35% by weight, from about 1% by weight to about 30% by weight, from about 1% by weight to about 25% by weight, from about 1% by weight to about 20% by weight, from about 1% by weight to about 15% by weight, from about 1% by weight to about 10% by weight, and/or from about 1% by weight to about 5% by weight compared with the weight of the polymer.

### Drug Molecules

Drug molecules include small molecules and biomolecules. Small molecules are low molecular weight organic compounds (typically less than about 2000 daltons). In some embodiments, the molecular weight of the drug molecule is in the range from about 300 to about 2000, from about 300 to about 1800, from about 300 to about 1600, from about 300 to about 1400, from about 300 to about 1200, from about 300 to about 1000, from about 300 to about 800, and/or from about 300 to about 600 daltons. Examples include cytochalasin B, gramicidin D, ethidium bromide, emetine, mitomycin, etoposide, tenoposide, colchicin, daunorubicin, dihydroxy anthracin dione, mithramycin, actinomycin D, 1-dehydrotestosterone, glucocorticoids, procaine, tetracaine, lidocaine, amphotericin B, propranolol, puromycin, maytansinoids, e.g., maytansinol (see U.S. Pat. No. 5,208,020), CC-1065 (see U.S. Pat. Nos. 5,475,092, 5,585,499, 5,846,545) and analogs or homologs thereof. Non-limiting examples of compositions of the present disclosure with a drug molecule payload are shown in FIGS. 51-50.

Other drug molecules include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), antifungal agents (e.g., butenafine, terbinafine, and naftifine), immunomodulating drugs (e.g., glatiramer acetate, fingolimod, teriflunomide, and dimethyl fumarate), and anti-mitotic agents (e.g., vincristine, vinblastine, paclitaxel, and maytansinoids).

Examples of suitable chemotherapeutic agents include any of: abarelix, aldesleukin, alitretinoin, allopurinol, altretamine, anastrozole, arsenic trioxide, asparaginase, azacitidine, bexarotene, bleomycin, bortezomib, busulfan, calusterone, capecitabine, carboplatin, carmustine, chlorambucil, cisplatin, cladribine, clofarabine, cyclophosphamide, cytarabine, dacarbazine, dactinomycin, dalteparin, dasatinib, daunorubicin, decitabine, denileukin, dexrazoxane, docetaxel, doxorubicin, dromostanolone, epirubicin, erlotinib, estramustine, etoposide, exemestane, filgrastim, floxuridine, fludarabine, fluorouracil, fulvestrant, gefitinib, gemcitabine, goserelin acetate, histrelin acetate, idarubicin, ifosfamide, imatinib, irinotecan, lapatinib ditosylate, lenalidomide, letrozole, leucovorin, leuprolide, levamisole, lomustine, meclorethamine, megestrol, melphalan, mercaptopurine, methotrexate, methoxsalen, mitomycin C, mitotane, mitoxantrone, nandrolone, nelarabine, nofetumomab, oxaliplatin, paclitaxel, pamidronate, pegaspargase, pegfilgrastim, pemetrexed, pentostatin, pipobroman, plicamycin, procarbazine, quinacrine, rasburicase, ruxolitinib, sorafenib, streptozocin, sunitinib, tamoxifen, temozolomide, teniposide, testolactone, thalidomide, thioguanine, thiotepa, topotecan, toremifene, tretinoin, uracil mustard, valrubicin, vinblastine, vincristine, vinorelbine, vorinostat, and zoledronate, or a pharmaceutically acceptable salt thereof.

Small molecules useful in the compositions and methods described herein bind with high affinity to a biopolymer, such as protein, nucleic acid, or polysaccharide, or other biological target. In one aspect, useful small molecules are capable of being functionalized by condensation with a carboxylic acid. For example, a small molecule can be an agent such as paclitaxel, which binds specifically to microtubules and is capable of being functionalized, e.g., with a carboxylic acid for attachment as an ester via a linker at the R¹ and/or R² position of Formula (I). Other examples include small molecules that bind specifically to receptors for hormones, cytokines, chemokines, or other signaling molecules, that may be encapsulated by a polymer of Formula (I). Small molecules include peptides.

The term “linker” as used herein refers to a group of atoms, e.g., 0-500, 0-10,000 atoms, and may be comprised of the atoms or groups such as, but not limited to, carbon, amino, alkylamino, oxygen, sulfur, sulfoxide, sulfonyl, carbonyl, and imine. The linker chain may also comprise part of a saturated, unsaturated or aromatic ring, including polycyclic and heteroaromatic rings wherein the heteroaromatic ring is an aryl group containing from one to four heteroatoms, N, O or S. Specific examples include, but are not limited to, unsaturated alkanes, polyethylene glycols, and dextran polymers. The linker must not interfere with binding of the ligand to the target.

In its simplest form, a linker can be a covalent chemical bond. In other embodiments, the linker can be a chemical group. Since the function of the linking group is merely to provide a physical connection, a wide variety of chemical groups can serve as linking groups. A linker is typically a divalent organic linking group where one valency represents the point of attachment to ligand or payload molecule and one valency represents the attachment to the polymer. The only requirement for the linker is to provide a stable physical linkage that is compatible with maintaining the function of the ligand or payload molecule and is compatible with the chemistry.

Examples of suitable linking groups include, e.g.: —O—, —S—, —S(O)—, —(O)₂—, —(O)—, —NH—, —N(C₁₋₆)alkyl, —NHC(O)—, —C(O)NH—, —O(CO)—, —C(O)O—, —O(CO)NH—, —NHC(O)O—, —O(CO)O—, —NHC(O)—NH—, —O(C₁₋₆)alkylene-, —S(C₁₋₆)alkylene-, —S(O)(C₁₋₆)alkylene-, —S(O)₂(C₁₋₆)alkylene-, —C(O)(C₁₋₆)alkylene-, —NH(C₁₋₆)alkylene)C(O)—, —C(O)(C₁₋₆)alkylene)C(O)—, —C(O)(C₁₋₆)alkylene)NH—, —O(CO)—, —C(O)O—, —O(CO)NH—, —NHC(O)—O—, —O(CO)O—, —NHC(O)—NH—, unsubstituted-(C₁₋₁₀)alkylene-, unsubstituted-(C₁₋₁₀)heteroalkylene, or —(C₁₋₁₀)alkylene or —(C₁₋₁₀)heteroalkylene substituted with one or more (e.g., 1, 2, 3, 4 or 5 substituents) independently selected from the group consisting of (C₁₋₆)alkyl, (C₂₋₆)alkenyl, (C₂₋₆)alkynyl, halogen, (C₁₋₆)haloalkyl, —CN, —NO₂, —C(═O)R, —OC(═O)Ar, —C(═O)OR, —C(═O)NR₂, —C(═NR)NR₂, —OR, —Ar, —OAr, —((C₁₋₆)alkylene)Ar, —O((C₁₋₆)alkylene)Ar, —OC(═O)C₁₋₆)alkyl, —OC(═O)OC₁₋₆)alkyl, —OC(═O)NR₂, —NR₂, —NRAr, —NR(C₁₋₆)alkylene)Ar, —NRC(═O)R, —NRC(═O)Ar, —NRC(═O)O(C₁₋₆)alkyl, —NRC(═O)NR₂, —NRSO₂R, —SR, —S(O)R, —SO₂R, —OSO₂(C₁₋₆)alkyl, —SO₂NR₂, (C₁₋₈)perfluoroalkyl, —(C₂₋₆)alkylene—OR, —O(C₂₋₆)alkylene—N(C₁₋₆)alkyl)₂, —P(═O)(OR)₂, —OP(═O)(OR)₂, oxo and sulfido, wherein each R group is hydrogen or (C₁₋₆alkyl), e.g., methyl and wherein each Ar is independently unsubstituted aryl or heteroaryl or aryl or heteroaryl substituted with one or more of (C₁₋₆)alkyl, (C₂₋₆)alkenyl, (C₂₋₆)alkynyl, halogen, (C₁₋₆)haloalkyl, —CN, —NO₂, —C(═O)R, —C(═O)OR, —C(═O)NR₂, —C(═NR)NR₂, —OR, —OC(═O)C₁₋₆)alkyl, —OC(═O)O(C₁₋₆ )alkyl, —OC(═O)NR₂, —NR₂, —NRC(═O)R, —NRC(═O)O(C₁₋₆)alkyl, —NRC(═O)NR₂, —NRSO₂R, —SR, —S(O)R, —SO₂R, —OSO₂(C i-C6)alkyl, —SO₂NR₂, (C₁₋₈)perfluoroalkyl, —(C₂₋₆)alkylene—OR, —O(C₂₋₆)alkylene—N(C₁₋₆)alkyl)₂, —P(═O)(OR)₂, —OP(═O)(OR)2 wherein each R group is hydrogen or (C₁₋₆alkyl). In addition, —(C₁₋₁₀)alkylene- and —(C₁₋₁₀)heteroalkylene can be substituted by one or more oxo groups (C═O) and the nitrogen and sulfur atoms of a heteroalkylene group can optionally be oxidized (e.g., to form S(O), —(O)₂—, or N-oxide). Suitable heteroalkylene groups can include one or more 1,2-dioxyethylene units —(O—CH₂CH₂)ₙO—, where n is an integer, e.g., 1, 2, 3, 4 or 5). The —(C₁₋₁₀)alkylene- and —(C₁₋₁₀)heteroalkylene also include —(C₁₋₆)alkylene- and —(C₁₋₆)heteroalkylene; and —(C₁₋₃)alkylene- and —(C₁₋₃)heteroalkylene.

Biomolecules are organic molecules produced by living organisms, including large polymeric molecules such as polypeptides, proteins, polysaccharides, and nucleic acids as well as small molecules such as primary metabolites, secondary metabolites, and natural products. Specific biomolecule examples include, but are not limited to, estradiol, testosterone, cholesterol, and phosphatidylserine.

### Detectable Agents

Examples of detectable agents include various organic small molecules, inorganic compounds, nanoparticles, enzymes or enzyme substrates, fluorescent materials, luminescent materials, bioluminescent materials, chemiluminescent materials, radioactive materials, and contrast agents. Examples of suitable enzymes include horseradish peroxidase, alkaline phosphatase, β-galactosidase, or acetylcholinesterase; examples of suitable fluorescent materials include boron-dipyrromethene (BODIPY®), 4,4-difluoro-5,7-dimethyl-4-bora-3a,4a-diaza-s-indacene-3-propionic acid (BODIPY® FL), 6—((4,4-difluoro-1,3-dimethyl-5-(4-methoxyphenyl)-4-bora-3a,4a-diaza-s-indacene-2-propionyl)amino)hexanoic acid, succinimidyl ester (BODIPY® TRM-X), Oregon Green 88, 6—(((4,4-difluoro-5-(2-pyrrolyl)-4-bora-3a,4a-diaza-s-indacene-3-yl)styryloxy)acetyl)aminohexanoic acid, Nile red (9-diethylamino-5-benzo[a]phenoxazinone), succinimidyl ester (BODIPY® 650/665-X), 7-N,N-diethylaminocoumarin, VIVOTAG 680 (an amine-reactive near-infra-red fluorochrome, from VisEn Medical), umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹⁸F, ⁶⁷Ga, ^{81m}Kr, ⁸²Rb, ¹¹¹In, ¹²³I, ¹³³Xe, ²⁰¹Tl, ¹²⁵I, ³⁵S, ¹⁴C, or ³H, ^{99m}Tc (e.g,. as pertechnetate (technetate(VII), TcO₄) either directly or indirectly, or other radioisotope detectable by direct counting of radioemission or by scintillation counting. In some embodiments, the molecular weight of the detectable agent is in the range from about 300 to about 2000, from about 300 to about 1000, and/or from about 300 to about 600 daltons. In addition, contrast agents, e.g., contrast agents for MRI or NMR, for X-ray CT, Raman imaging, optical coherence tomography, absorption imaging, ultrasound imaging, or thermal imaging can be used. Exemplary contrast agents include gold, gadolinium (e.g., chelated Gd), iron oxides (e.g., superparamagnetic iron oxide (SPIO), monocrystalline iron oxide nanoparticles (MIONs), and ultrasmall superparamagnetic iron oxide (USPIO)), manganese chelates (e.g., Mn-DPDP), barium sulfate, iodinated contrast media (iohexol), microbubbles, or perfluorocarbons. In some embodiments, the detectable agent comprises gadolinium.

For example, a composition of the present disclosure may be used to deliver a payload of chelated Gd (e.g., Gd-DTPA) to a cancer tumor for MRI imaging. The payload may allow for greater uptake of the payload and imaging of, e.g., different areas within a cancer tumor, since the uptake of the composition would not be mediated by passive transport. A composition of the present disclosure may be taken up via active transport mechanisms (e.g., phagocytosis, pinocytosis) and thus may avoid issues of Pgp efflux, commonly exhibited by cancer cells, that would limit uptake of the chelated Gd.

### Metal Ions

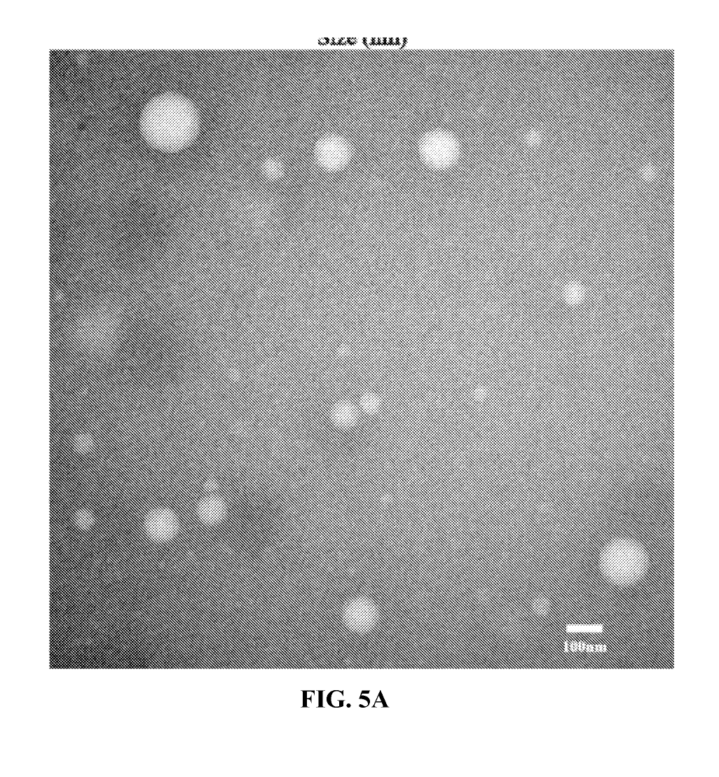

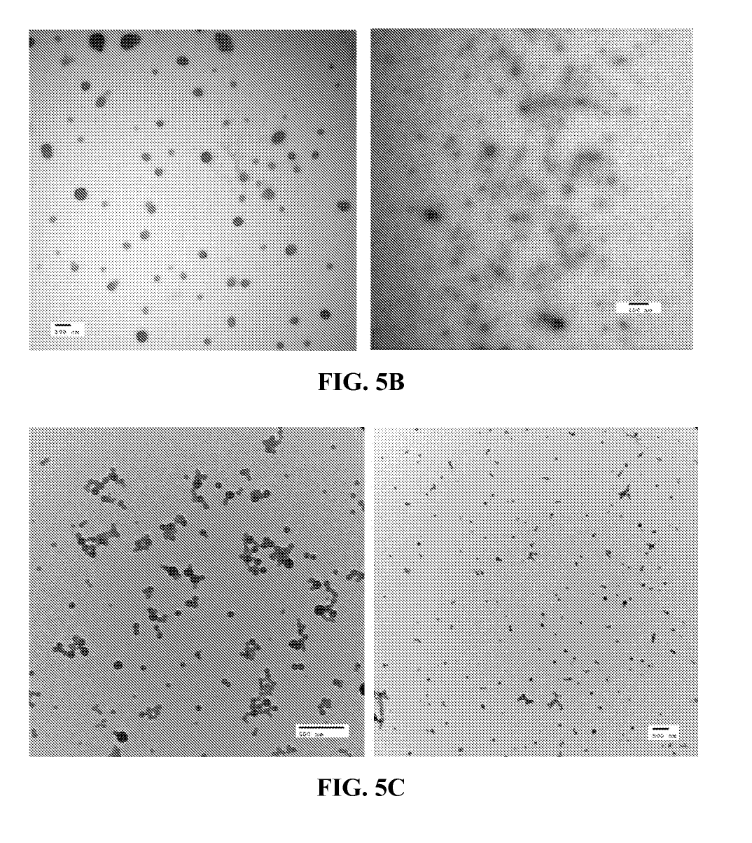

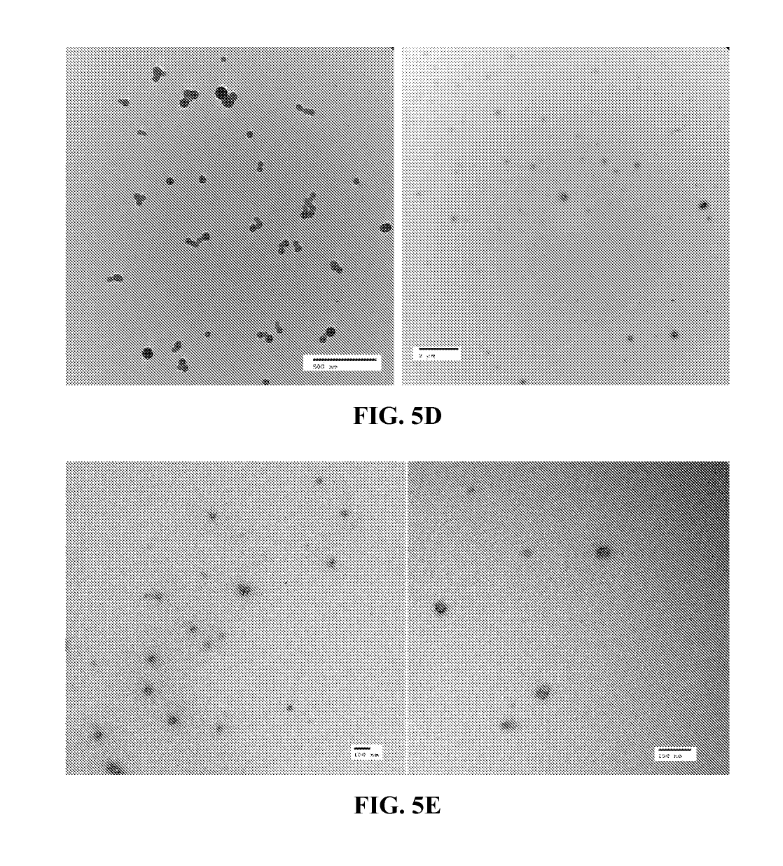

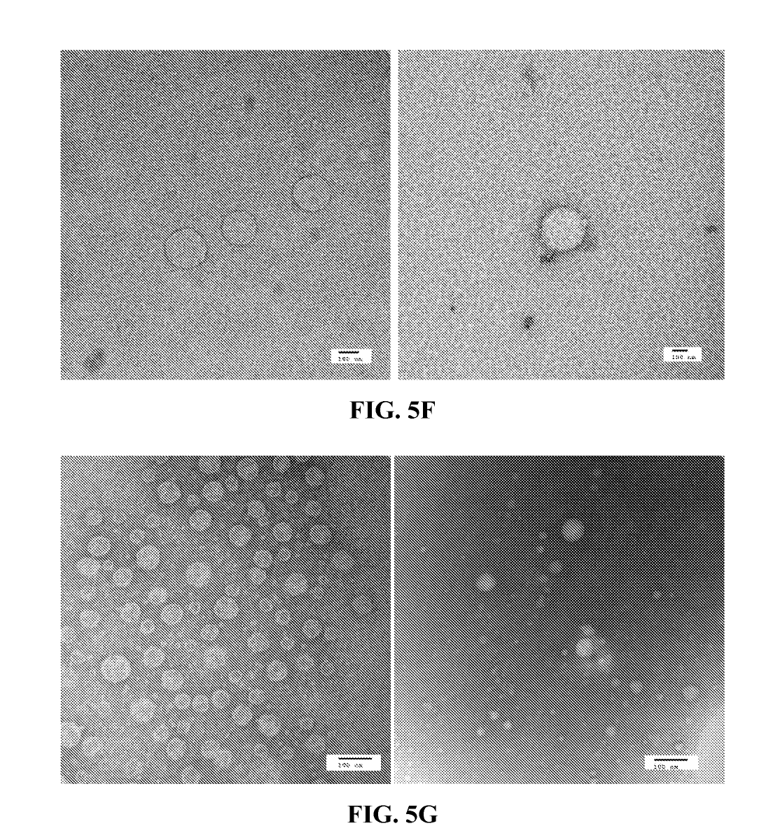

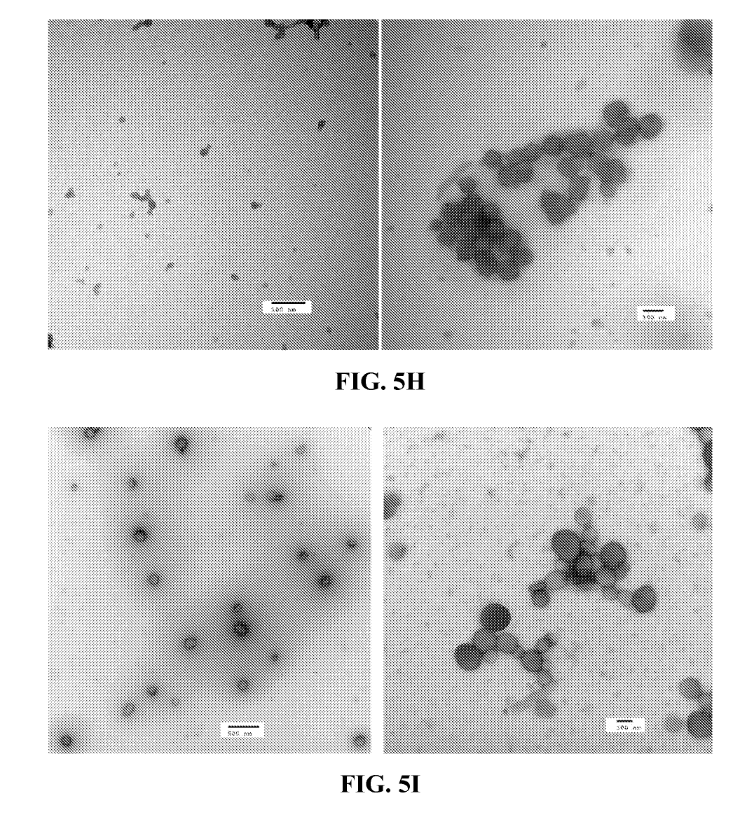

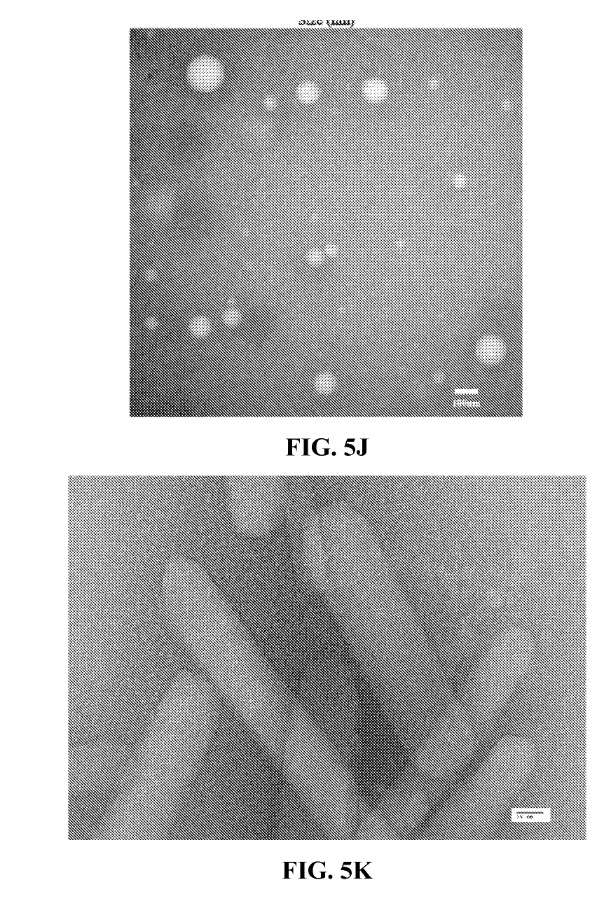

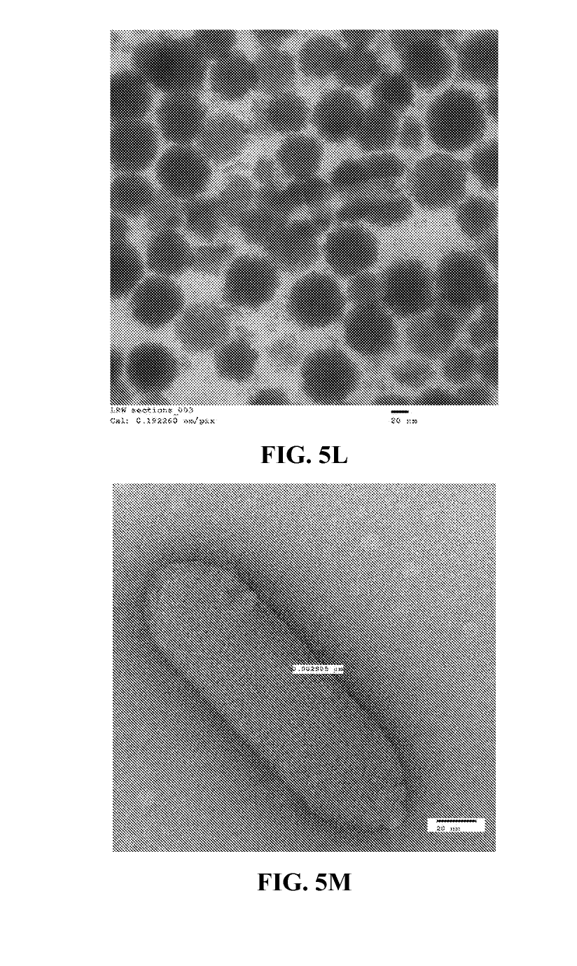

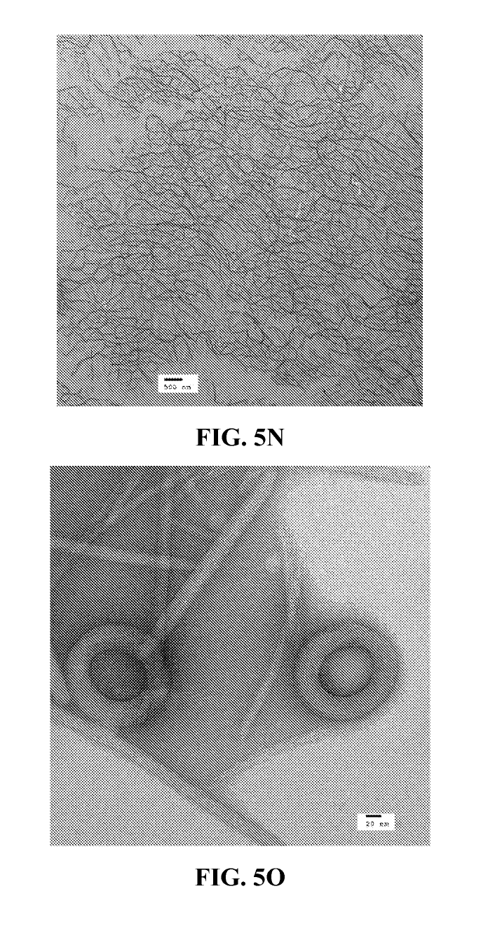

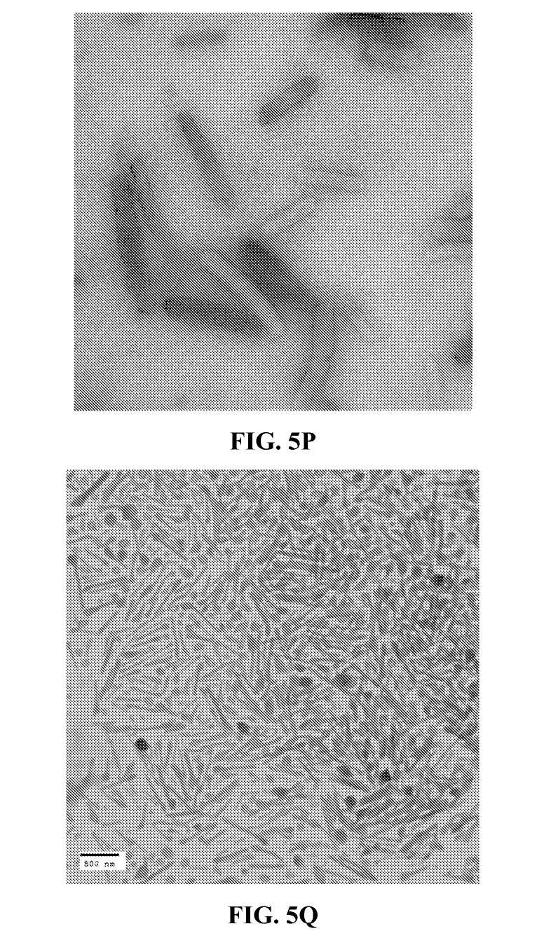

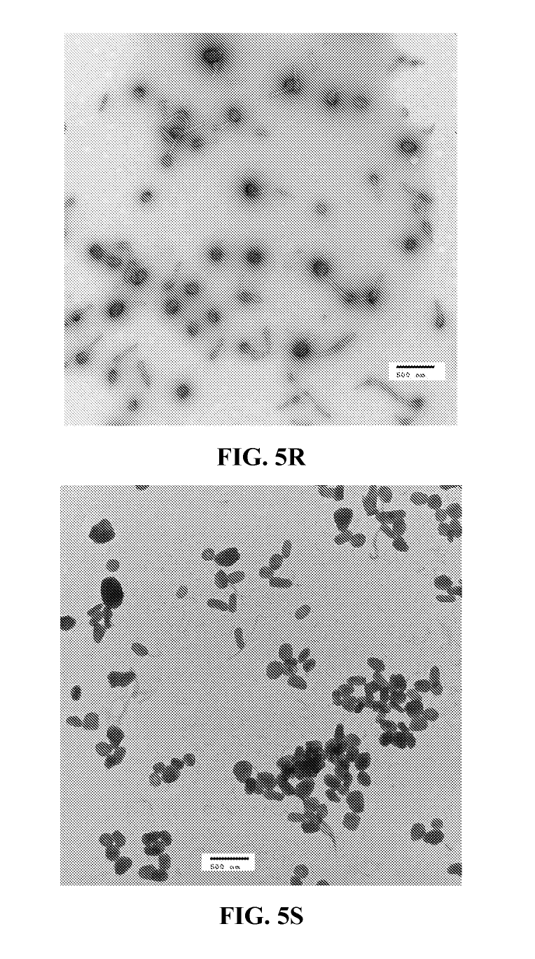

In one aspect, the payload is an inorganic salt, which can include a salt of an alkali metal, such as Li⁺, Na⁺, and K⁻; an alkaline earth metal, such as Mg²⁺, Ca²⁺, and Ba²⁺; a transition metal, such as Ag⁺, Cu⁺, Cu²⁺, Cr³⁺, Cr⁶⁺, Fe²⁺, and Fe³⁺; and other metal, such as Pb²⁺, La³⁺, Eu³⁻, and Gd³⁺. Examples of useful inorganic salts include GdCl₃, LiCl, FeCl₂, FeCl₃, CuCl₂, and AgNO₃. Some examples of compositions of the present disclosure with a metal ion payload are shown in FIGS. 5C-5H. In some embodiments, the payload is encapsulated.

In some embodiments, payload metal ion can be included in the polymers disclosed herein as a complex or chelate. For example, the payload can be included in a complex or chelate of a metal ion such as a transition metal, e.g., Ag⁺, with carboxylate groups of a polymer of Formula (I). In a non-limiting example, Gd⁺ can be useful for MRI imaging. In another non-limiting example, the payload can be included in a complex or chelate of a metal ion such as a transition metal, e.g., Cu²⁺, with the exposed amines of a polymer of Formula (II).

The ability to deliver a payload of metal ions across barriers, e.g., cell membranes, would be useful for a number of applications. For example, Ag⁺ or Cu⁺ can be useful for antibacterial applications. In another non-limiting example, Li⁺ ion transport would be useful in the development of alternative batteries. Further, the ability to entrap or collect metal ions such as Cr⁶⁺ or Pb²⁺would be useful for pollution abatement.

### Controlled Release

In some embodiments, the compositions are formulated for controlled-release. In some embodiments, controlled release can be achieved by implants. Nanoparticle compositions can release the active agent (e.g., a drug molecule or detectable agent) through surface or bulk erosion, diffusion, and/or swelling followed by diffusion, in a time- or condition-dependent manner. The release of the active agent(s) can be constant over a long or short period, it can be cyclic over a long or short period, or it can be triggered by the environment or other external events (see, e.g., Langer and Tirrell, Nature 428:487-492, 2004). For therapeutic uses, controlled-release polymer systems can provide drug levels in a specific range over a longer period of time than other drug delivery methods, thus increasing the efficacy of the drug and maximizing patient compliance.

### Formulations

### Stabilizers

To provide additional stability, in some embodiments, a polymer, a nanoparticle, or a composition of the present disclosure may be protected with a stabilizer, such as a coating, for example, PVA (MW), Tween® 80, Pluronic® (e.g., F127, F68, etc.), PEG, MYRJ™, lipid, or lipid-PEG. Specific coating stabilizers include DSPE-PEG3000/Lipid and PLGA50K/DSPE-PEG3000, and are well-known in the art. Non-limiting examples of compositions of the present disclosure with lipid coating are shown in FIGS. 5P and 5Q.

### Additional Ingredients

A composition may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars and the like into the compositions. In addition, prolonged absorption of the injectable pharmaceutical form may be brought about by the inclusion of agents which delay absorption such as aluminum monostearate and gelatin.

Compositions containing a polymer of Formula (I), Formula (Ia), and/or Formula (II) and a drug molecule or a detectable agent and prepared as described herein can be administered in various forms, depending on the disease or disorder to be treated and the age, condition, and body weight of the subject, as is well known in the art. For example, where the compositions are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These formulations can be prepared by conventional means in conjunction with the methods described herein, and, if desired, the active ingredient may be mixed with any conventional additive or excipient, such as a binder, a disintegrating agent, a lubricant, a corrigent, a solubilizing agent, a suspension aid, an emulsifying agent, or a coating agent.

A tablet may be made by compression or molding, optionally with one or more accessory ingredients. Compressed tablets may also be prepared using inert diluent, preservative, disintegrant (for example, sodium starch glycolate or cross-linked sodium carboxymethyl cellulose), surface-active or dispersing agent. Molded tablets may be made by molding in a suitable machine a mixture of a powdered compound moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes.

Liquid dosage forms for oral administration include pharmaceutically acceptable emulsions, microemulsions, solutions, suspensions, syrups, and elixirs. In addition to the polymer of Formula (I), Formula (Ia), and/or Formula (II) and a drug molecule or a detectable agent, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Suspensions, in addition to the polymer and a drug molecule or a detectable agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for parenteral administration can include a polymer and a drug molecule or a detectable agent as provided herein in combination with one or more pharmaceutically acceptable sterile aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions provided herein include water for injection (e.g., sterile water for injection), bacteriostatic water, ethanol, polyols (such as glycerol, propylene glycol, polyethylene glycol such as liquid polyethylene glycol, and the like), sterile buffer (such as citrate buffer), and suitable mixtures thereof, vegetable oils, such as olive oil, injectable organic esters, such as ethyl oleate, and Cremophor EL™ (BASF, Parsippany, N.J.). In all cases, the composition must be sterile and should be fluid to the extent that easy syringability exists. Proper fluidity can be maintained, for example, by the use of coating materials, such as lecithin, by the maintenance of the required particle size in the case of dispersions, and by the use of surfactants.

The composition should be stable under the conditions of manufacture and storage and must be preserved against the contaminating action of microorganisms such as bacteria and fungi. Prolonged absorption of the injectable compositions can be brought about by including in the composition an agent that delays absorption, for example, aluminum monostearate and gelatin.

Sterile injectable solutions can be prepared by incorporating the polymer and drug molecule or detectable agent in the required amounts in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation is freeze-drying (lyophilization), which yields a powder of the active ingredient plus any additional desired ingredient from a previously sterile-filtered solution thereof.

For administration by inhalation, the polymer and drug molecule or detectable agent can be delivered in the form of an aerosol spray from a pressured container or dispenser that contains a suitable propellant, e.g., a gas such as carbon dioxide, or a nebulizer. Such methods include those described in U.S. Pat. No. 6,468,798. Additionally, intranasal delivery can be accomplished, as described in, inter alia, Hamajima et al., Clin. Immunol. Immunopathol., 88(2), 205-10 (1998).

Ordinarily, an aqueous aerosol can be made by formulating an aqueous solution or suspension of the agent together with conventional pharmaceutically acceptable carriers and stabilizers. The carriers and stabilizers vary with the requirements of the particular composition, but typically include nonionic surfactants (polysorbates, e.g., TWEEN®; poloxamers, e.g., PLURONIC®; sorbitan esters; lecithin; and polyethoxylates, e.g., CREMOPHOR®), pharmaceutically acceptable co-solvents such as polyethylene glycol, innocuous proteins like serum albumin, sorbitan esters, oleic acid, lecithin, amino acids such as glycine, buffers, salts, sugars, or sugar alcohols. Aerosols generally are prepared from isotonic solutions.

Systemic administration of a composition as described herein can also be by transmucosal or transdermal means. Dosage forms for the topical or transdermal administration of a compound provided herein include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the compositions are formulated into ointments, salves, gels, or creams as generally known in the art.

The ointments, pastes, creams, and gels may contain, in addition to one or more polymers provided herein, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a polymer of Formula (I), Formula (Ia), and/or Formula (II) provided herein, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

The pharmaceutical compositions can also be prepared in the form of suppositories or retention enemas for rectal and/or vaginal delivery. Formulations presented as a suppository can be prepared by mixing one or more compounds provided herein with one or more suitable nonirritating excipients or carriers comprising, for example, cocoa butter, glycerides, polyethylene glycol, a suppository wax or a salicylate, which is solid at room temperature, but liquid at body temperature and, therefore, will melt in the rectum or vaginal cavity and release the active agent. Formulations which are suitable for vaginal administration also include pessaries, tampons, creams, gels, pastes, foams, or spray formulations containing such carriers as are known in the art to be appropriate.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection, and infusion.

The phrases “systemic administration”, “administered systemically”, “peripheral administration”, and “administered peripherally” as used herein mean the administration of a composition via route other than directly into the central nervous system, such that it enters the subject's system and thus, is subject to metabolism and other like processes, for example, subcutaneous administration.

Actual dosage levels of the active ingredients in the compositions provided herein may be varied so as to obtain an amount of the active ingredient which is effective to achieve the desired therapeutic response for a particular subject, composition, and mode of administration, without being toxic to the subject.

The concentration of a drug molecule or detectable agent provided herein in a pharmaceutically acceptable composition will vary depending on several factors, including the dosage of the compound to be administered, the pharmacokinetic characteristics of the drug molecule(s) employed, and the route of administration. In some embodiments, the compositions provided herein can be provided in an aqueous solution containing about 0.1-10% w/v of a drug molecule or detectable agent disclosed herein, among other substances, for parenteral administration. Typical dose ranges can include from about 0.01 to about 50 mg/kg of body weight per day, given in 1-4 divided doses. Each divided dose may contain the same or different drug molecules. The dosage will be a therapeutically effective amount depending on several factors including the overall health of a subject, and the formulation and route of administration of the selected drug molecule(s).

Dosage forms or compositions containing a drug molecule or detectable agent as described herein in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%. Although the dosage will vary depending on the symptoms, age and body weight of the subject, the nature and severity of the disorder to be treated or prevented, the route of administration and the form of the drug or agent, in general, a daily dosage of from 0.01 to 2000 mg of the compound is recommended for an adult human subject, and this may be administered in a single dose or in divided doses. The amount of active ingredient which can be combined with a carrier material to produce a single dosage form will generally be that amount of the compound which produces a therapeutic effect.

The pharmaceutical composition may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

The precise time of administration and/or amount of the composition that will yield the most effective results in terms of efficacy of treatment in a given subject will depend upon the activity, pharmacokinetics, and bioavailability of a particular drug molecule, physiological condition of the subject (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the subject and adjusting the dosage and/or timing.

The compositions can be included in a container, pack, or dispenser together with instructions for administration.

### III. Methods of Use

Provided in the disclosure is a method of transporting a drug molecule in a cell, comprising contacting the cell with a composition as described herein. In some embodiments, the contacting is in vitro, for example, in a diagnostic assay. In some embodiments, the contacting is in vivo. In some embodiments, the cell is an ex vivo culture. In some embodiments, the cell is grown in a tissue culture medium. In some embodiments, the cell is a mammalian cell. For example, the mammalian cell can be a human cell. In some embodiments, the cell is a cancer cell.

In some embodiments, the cancer cell is selected from a breast cancer cell, a colon cancer cell, a leukemia cell, a bone cancer cell, a lung cancer cell, a bladder cancer cell, a brain cancer cell, a bronchial cancer cell, a cervical cancer cell, a colorectal cancer cell, an endometrial cancer cell, an ependymoma cancer cell, a retinoblastoma cancer cell, a gallbladder cancer cell, a gastric cancer cell, a gastrointestinal cancer cell, a glioma cancer cell, a head and neck cancer cell, a heart cancer cell, a liver cancer cell, a pancreatic cancer cell, a melanoma cancer cell, a kidney cancer cell, a laryngeal cancer cell, a lip or oral cancer cell, a lymphoma cancer cell, a mesothioma cancer cell, a mouth cancer cell, a myeloma cancer cell, a nasopharyngeal cancer cell, a neuroblastoma cancer cell, an oropharyngeal cancer cell, an ovarian cancer cell, a thyroid cancer cell, a penile cancer cell, a pituitary cancer cell, a prostate cancer cell, a rectal cancer cell, a renal cancer cell, a salivary gland cancer cell, a sarcoma cancer cell, a skin cancer cell, a stomach cancer cell, a testicular cancer cell, a throat cancer cell, a uterine cancer cell, a vaginal cancer cell, and a vulvar cancer cell.

Also provided is a method of treating a disease or condition in a subject in need thereof, comprising administering to the subject a therapeutically effective amount of a polymer, a nanoparticle, or a composition as described herein.

Also provided is a method of imaging a disease or condition in a subject, comprising administering to the subject a composition as described herein.

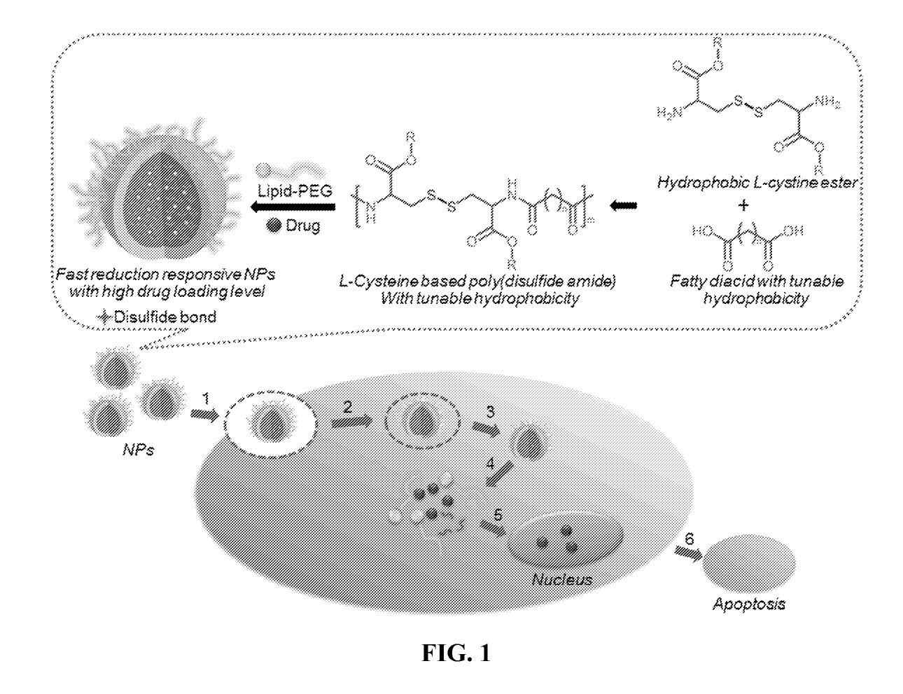

While not being limited by any theory, it is believed that the compositions described herein can be useful for the treatment of conditions characterized by hypoxic cellular conditions such as cancer. Hypoxia is a feature of cancer cells and significantly alters local redox microenvironments within tumor tissue. The intracellular levels of glutathione can be 1001000 fold higher in cancer cells than in normal tissue, and redox-sensitive drug release may be useful for delivery of therapeutic molecules to cancer cells. Further, hypoxic characteristics play a critical role in tumor progression in such areas as tumor invasion, metastasis, and therapeutic resistance, so redox-sensitive drug release can provide added therapeutic benefits. While not being limited by any theory, the disulfide based nanoparticle systems described herein can be useful for drug delivery. The compositions can be stable in the blood stream but can allow for rapid drug release upon reduction of disulfide bonds after cell uptake. A scheme showing delivery of a drug, e.g., docetaxel, for the treatment of a hypoxic condition, e.g., cancer using according to the invention is shown in FIG. 1. For example, cysteine-based copolymer with tunable hydrophobicity as described herein can be used to prepare reduction-responsive nanoparticles which can be loaded with a drug, e.g., docetaxel. The ability to tune the hydrophobicity and degradability of the nanoparticles (e.g., by altering the diacid repeating unit) allows designing nanoparticle systems suitable for drug delivery. When nanoparticles are taken up by hypoxic cells (e.g., cancer cells), the nanoparticle structure can be degraded through reduction of the disulfide bonds, leading to drug release into the cell where the drug can exert its therapeutic effect such as causing apoptosis of cancer cells.

In some embodiments, the subject is a mammal. The mammal can be a farm animal, such as a donkey, a goat, a sheep, a cow, or a pig; a veterinary animal, such as a cat, a dog, a rat, a mouse; or a primate, such as a monkey (e.g., a cynomolgus monkey), a baboon, or a human.

In some embodiments, the disease or condition is a cancer. For example, the cancer can be selected from kidney, ovarian, breast, prostate, brain, esophageal, stomach, pancreatic, adrenal, skin, uterine, cervical, bladder, testicular, colon, anal, liver, bile duct, lung, thyroid cancers, head and neck cancer, sarcoma, leukemia, lymphoma, and myeloma.

Also provided is a method of hair restructuring in a subject, comprising administering to the subject a polymer, a nanoparticle, or a composition as described herein.

Also provided is a method of skin restructuring in a subject, comprising administering to the subject a polymer, a nanoparticle, or a composition as described herein for use as a skin adhesive.

Also provided is a method of transporting a metal ion. In some embodiments, the metal ion is selected from Cu⁻, Cu²⁺, Fe²⁺, Fe³⁺, Cr³⁺, Cr⁶⁺, Pb²⁺, Ag⁺, Gd³⁺, and Li⁺. In some embodiments, the metal ion is, e.g., a Ag₊ or Cu⁺ ion, for antibacterial use. In some embodiments, the metal ion is, e.g., a Pb²⁺ or Cr⁶⁺ ion, for use in pollution treatment. In some embodiments, the metal ion is, e.g., a Li⁺ ion, for use in a battery. In some embodiments, the metal ion is present as an inorganic salt.

### EXAMPLES

The invention is further described in the following examples, which do not limit the scope of the invention described in the claims.

### Materials

L-Cysteine dimethyl ester dihydrochloride ((H-Cys-OMe)2.2HC1), succinyl chloride, adipoyl chloride, suberoyl chloride, sebacoyl chloride, dodecanedioyl dichloride, p-nitrophenol, triethylamine, and alamar Blue were purchased from VWR Scientific (West Chester, Pa.) and used without further purification. Pluronic® F-127, poly(vinyl alcohol) (PVA, MW 13,000-23,000), Tween® 80, poly(oxyethylene stearate) (MYRJ™) and dithiothreitol (DTT), tris(2-carboxyethyl)phosphine (TCEP), glutathione tripeptide (γ-glutamyl-cysteinyl-glycine, GSH), and fluorescence dyes (Nile red and Coumarin 6) were all purchased from Sigma-Aldrich (St. Louis, Mo.) and used without further purification. Lipid (such as lecithin) and lipid-PEG (such as 1,2-distearoyl-sn-glycero-3-phosphoethanolamine-poly(ethylene glycol) (DSPE-PEG)) were purchased from Avanti Polar Lipids. 50:50 Poly(DL-lactide-co-glycolide)(PLGA, B6013-2) was purchased from LACTEL absorbable polymers (Birmingham, Ala.). Docetaxel (Dtxl) was purchased from LC laboratories. Organic solvents like acetone, dimethyl sulfoxide (DMSO), ethyl acetate, ethyl ether, methanol, 2-propanol, and toluene were purchased from Sigma-Aldrich (St. Louis, Mo.) and purified by standard methods before use. Other chemicals and reagents if not otherwise specified were purchased from VWR Scientific (West Chester, Pa.).

### Measurements

The physicochemical properties of the polymers and nanoparticles were characterized by various standard methods. ¹H NMR spectra were recorded with a Varian Unity Inova 400-MHz spectrometer (Palo Alto, Calif.). Deuterated dimethyl sulfoxide (DMSO-d6) (Cambridge Isotope Laboratories, Andover, Mass.) with tetramethylsilane as an internal standard was used as the solvent. MestReNova software was used for data analysis. Elemental analyses of the polymers/nanoparticles were performed with a PE 2400 CHN elemental analyzer by Atlantic Microlab (Norcross, Ga.). The thermal property of the polymers was characterized with a DSC 2920 (TA Instruments, New Castle, DE). The measurements were carried out from -30.0 to 150.0° C. at a heating rate of 10.0° C·min.⁻¹ and at a nitrogen gas flow rate of 25.0 mL·min.⁻¹ TA Universal Analysis software was used for thermal data analysis. The solubility of polymers in common organic solvents at room temperature was assessed by using 1.0 mg·mL⁻¹ as the solubility criterion. For molecular weight (Mₙ) measurement, polymers were dissolved at a concentration of 1.0 mg·mL⁻¹ in THF solution and measured by gel permeation chromatography (GPC). The nanoparticle sizes and ζ-potentials were obtained by quasi-electric laser light scattering using a ZetaPALS dynamic light-scattering detector (15 mW laser, incident beam ¼ 676 nm; Brookhaven Instruments). Transmission electron microscopy (TEM) was performed at the Harvard Medical School EM facility on a Tecnai G2 Spirit BioTWIN TEM. A fluorescence microscope (Zeiss Axiovert 200) was used to record the intracellular activity of nanoparticles. Fluorescent dye release was measured by Synergy HT Multi-Mode Microplate Reader (Biotek Instruments). Docetaxel drug release was measured by HPLC (Agilent Technologies, 1200 series).

### Cell Culture

Cells (Hela, DU145, A549, MCF-7) were purchased from American Tissue Culture Collection (ATCC). The cells were grown by recommended protocol from the manufacturer. For example, Hela cells were grown at 37° C. in 5% CO₂ in Dulbecco's minimal essential medium (DMEM) supplemented with 10% FBS (Invitrogen, Carlsbad, Calif.) and antibiotics. Hela cells were used from passages 6-12, and medium was changed every two days. Cells were grown to 70% confluence before splitting or harvesting.

### Statistics

When appropriate, data are presented as mean± standard error of the mean calculated over at least three data points. Significant differences compared to control groups were evaluated by unpaired Student's t-test or Dunnet test at p<0.05, and between more than two groups by Tukey's test with or without one-way ANOVA analysis of variance. JMP software (version 8.0, from SAS Company) was used for data analysis.

### Example 1

### Synthesis of Cysteine-based Poly(Disulfide Amide)s (Cys-PDSA)

Cys-poly(disulfide amide) (Cys-PDSA) polymers were prepared by one-step polycondensation of (H-Cys-OMe)₂·2HCl and bis-fatty acid nitrophenol ester or dichloride of fatty acid in a variety of combinations (Scheme 3). Prepared PDSAs are labeled as Cys-OMe-x or, equivalently Cys-xE, where x represents the number of methylene groups in the diacid repeating unit. These notations are used interchangeably throughout the present disclosure. Accordingly, the cysteine dimethyl ester copolymer with the respective blocks are coded as follows: succinyl chloride (Cys-OMe-2 or Cys-2E), adipoyl chloride (Cys-OMe-4 or Cys-4E), suberoyl chloride (Cys-OMe-6 or Cys-6E), sebacoyl chloride (Cys-OMe-8, or Cys-8E), and dodecanedioyl dichloride (Cys-OMe-10 or Cys-10E). The corresponding carboxylic acid polymers are coded with the cysteine carboxylic acid copolymer with the respective blocks as follows: succinyl chloride (Cys-OH-2), adipoyl chloride (Cys-OH-4), suberoyl chloride (Cys-OH-6), sebacoyl chloride (Cys-OH-8), and dodecanedioyl dichloride (Cys-OH-10).

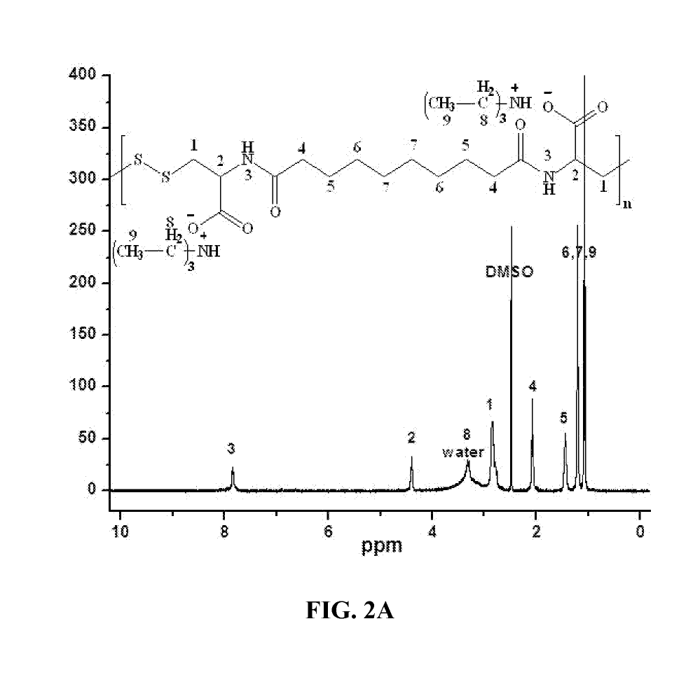

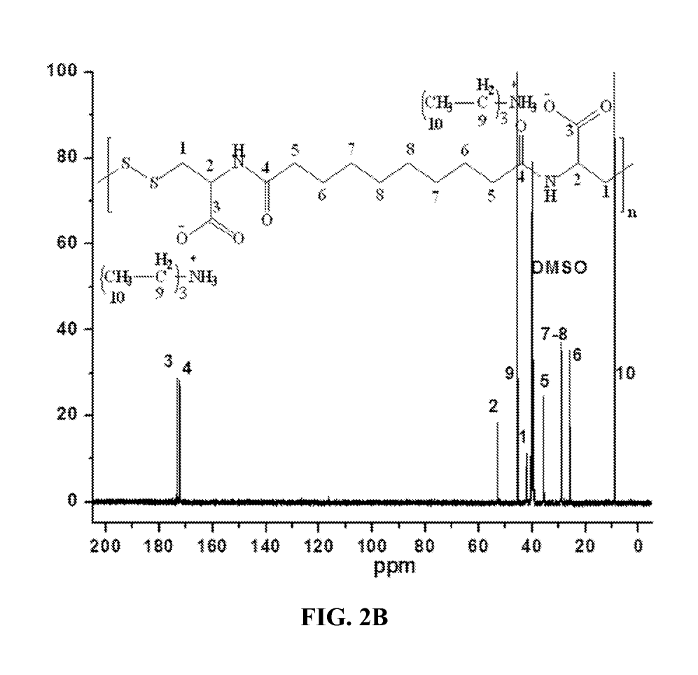

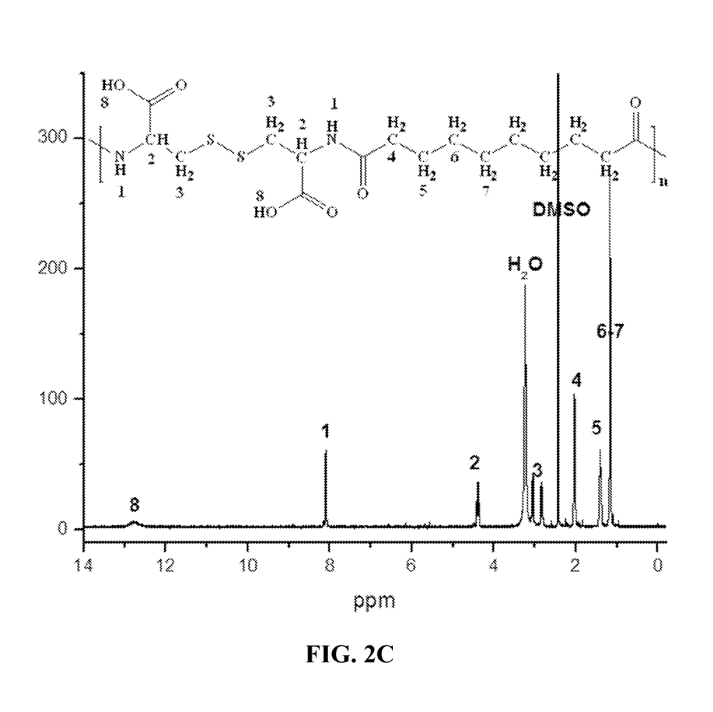

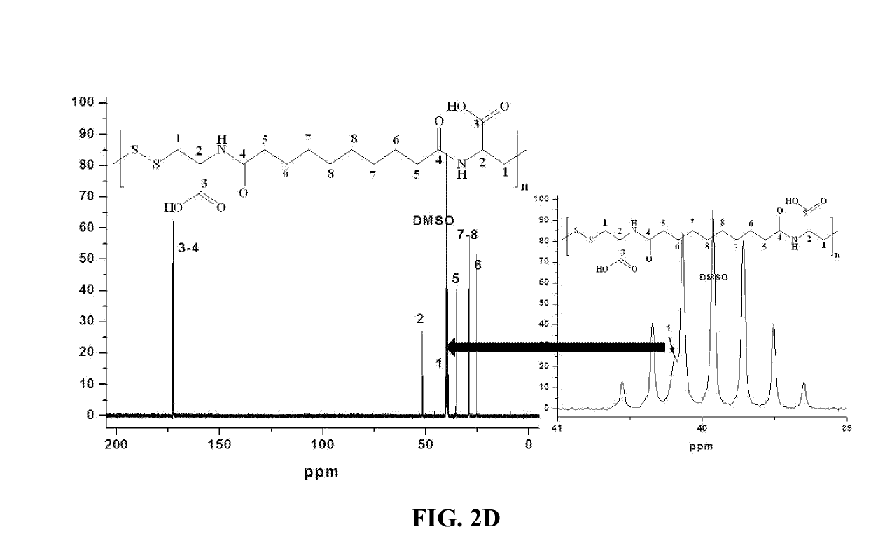

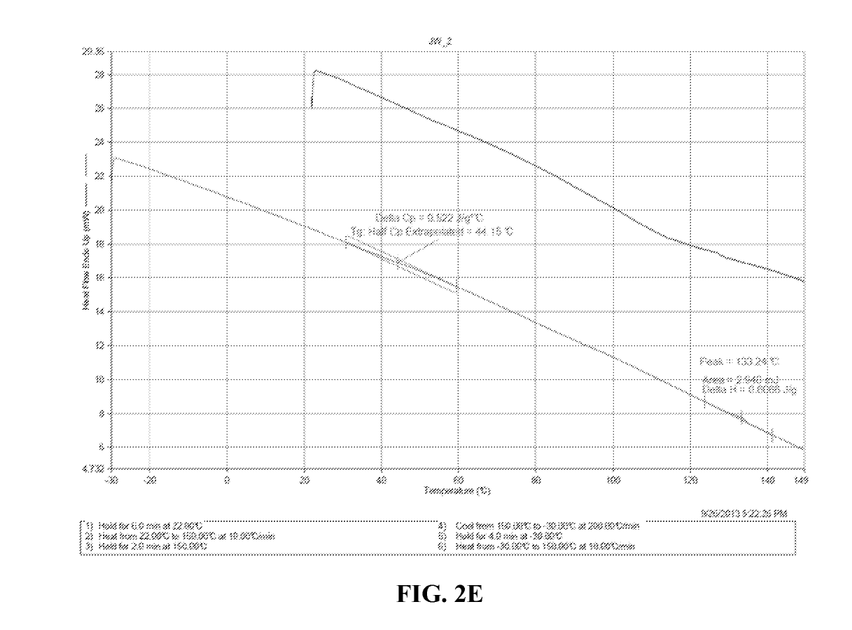

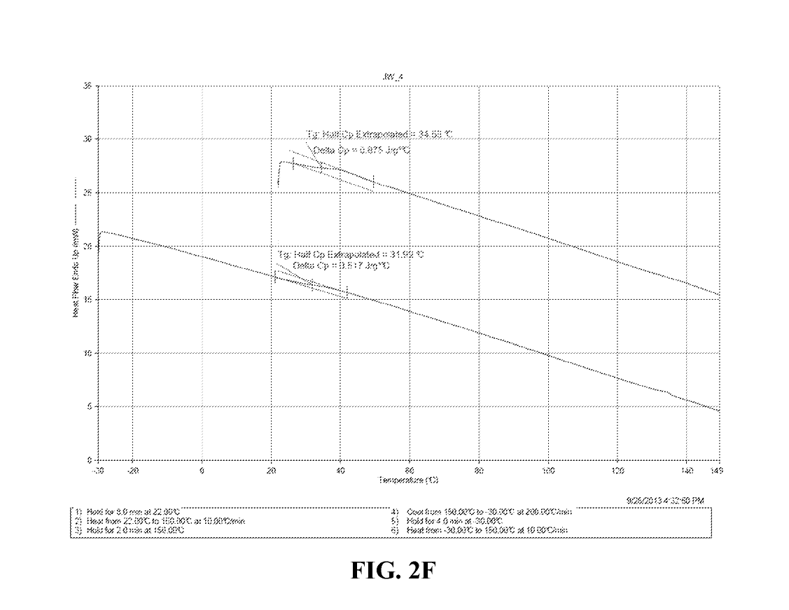

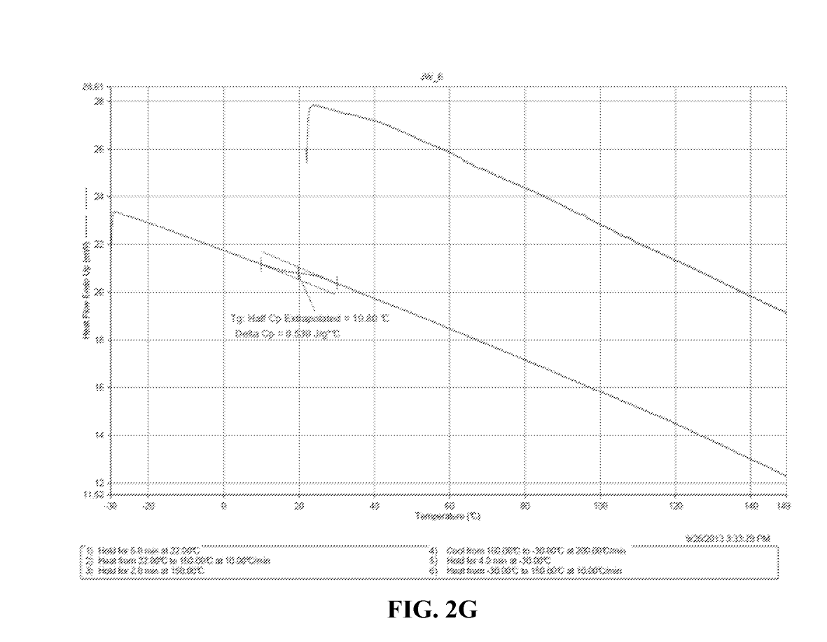

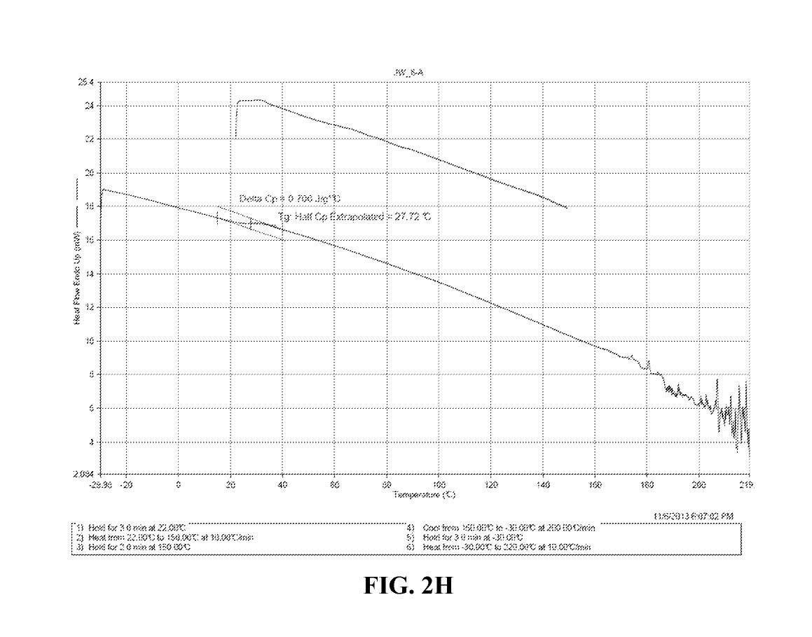

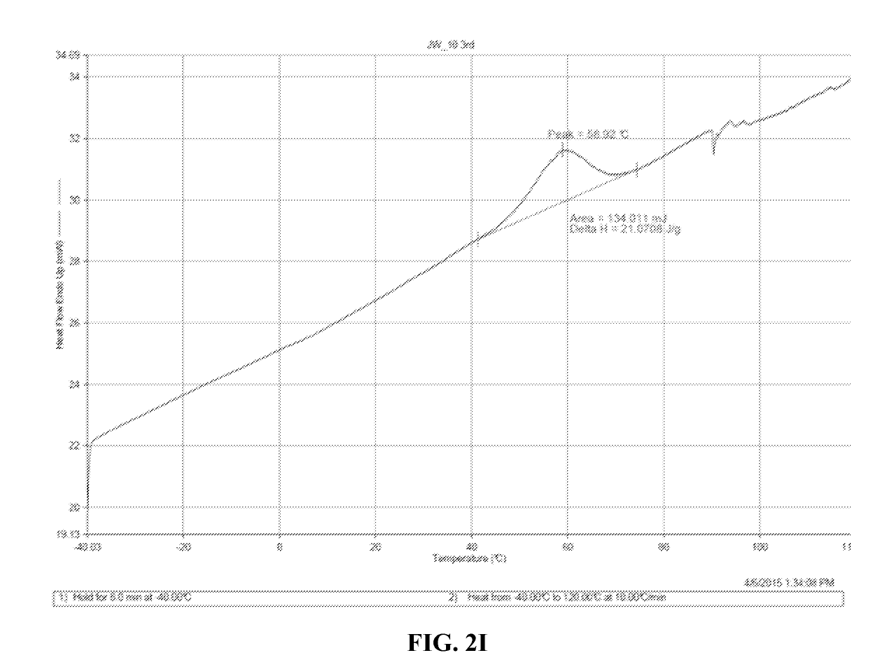

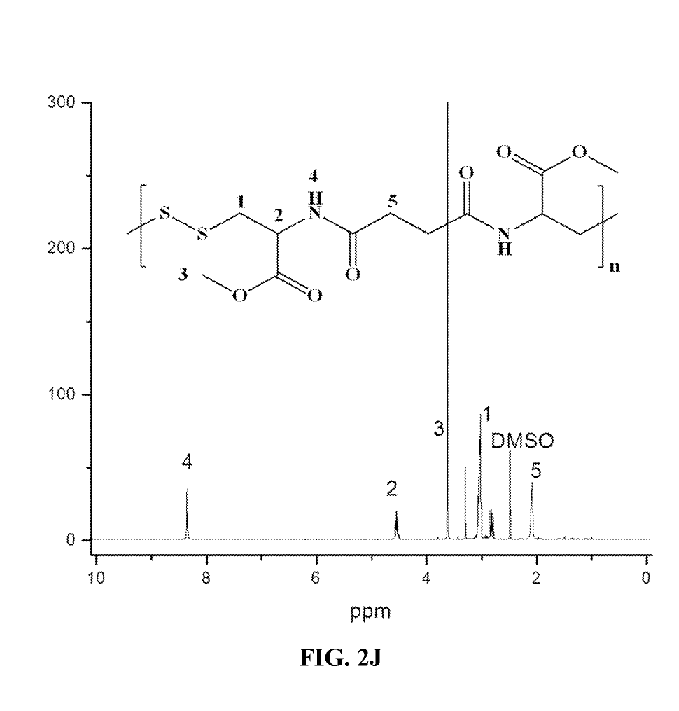

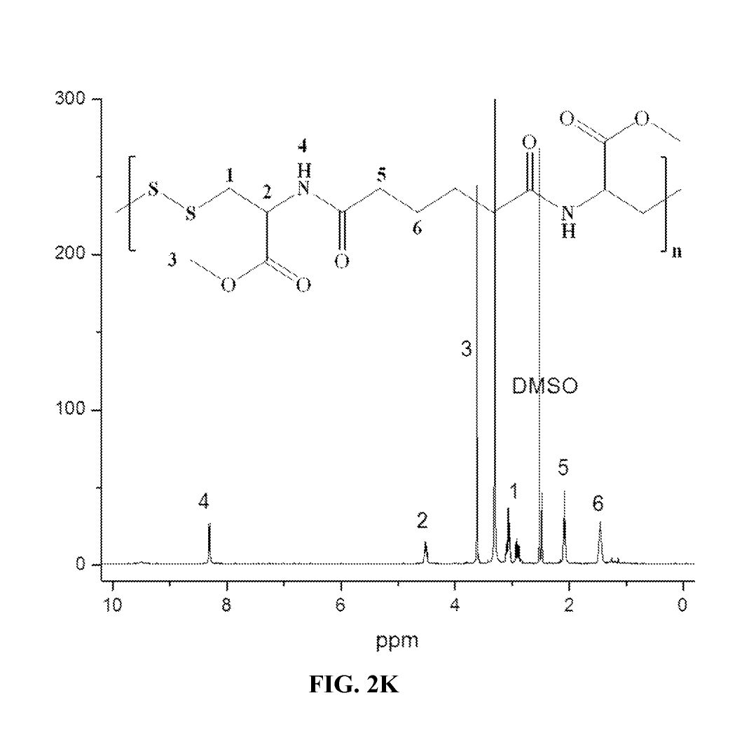

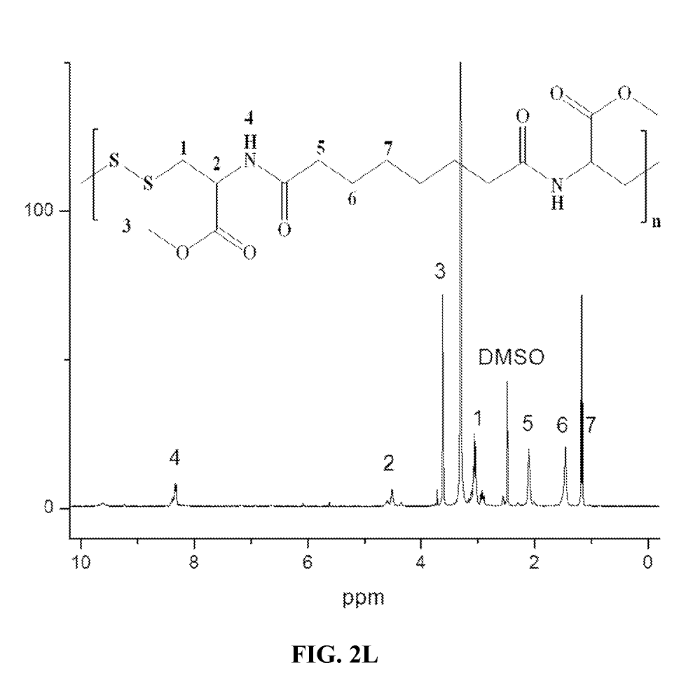

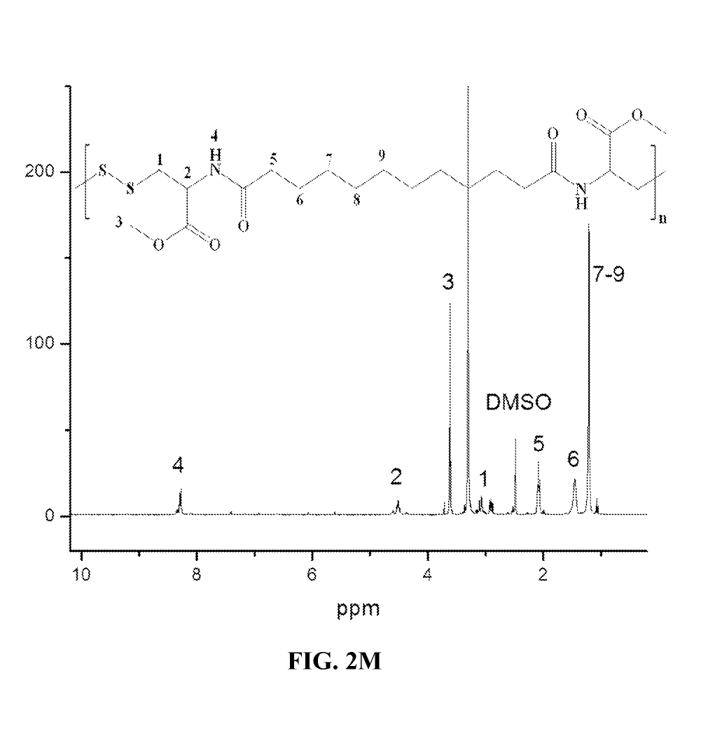

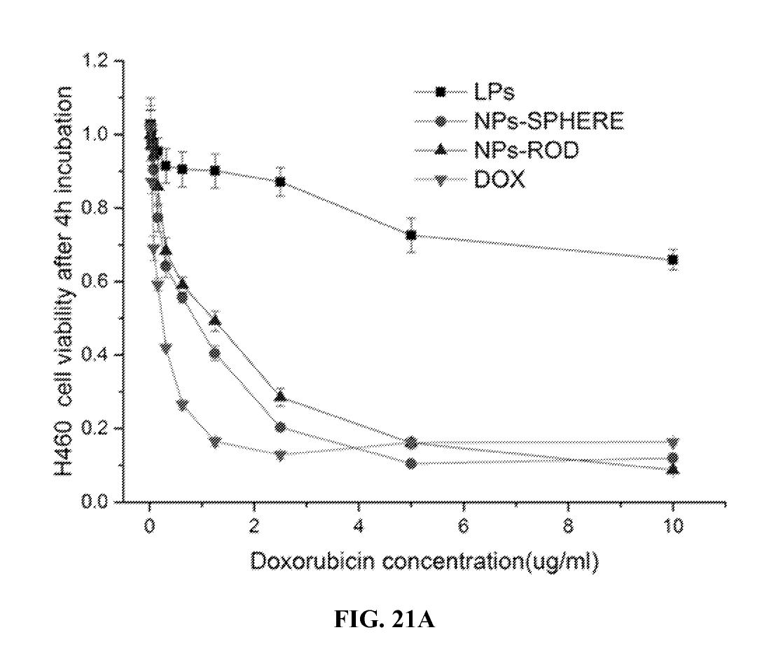

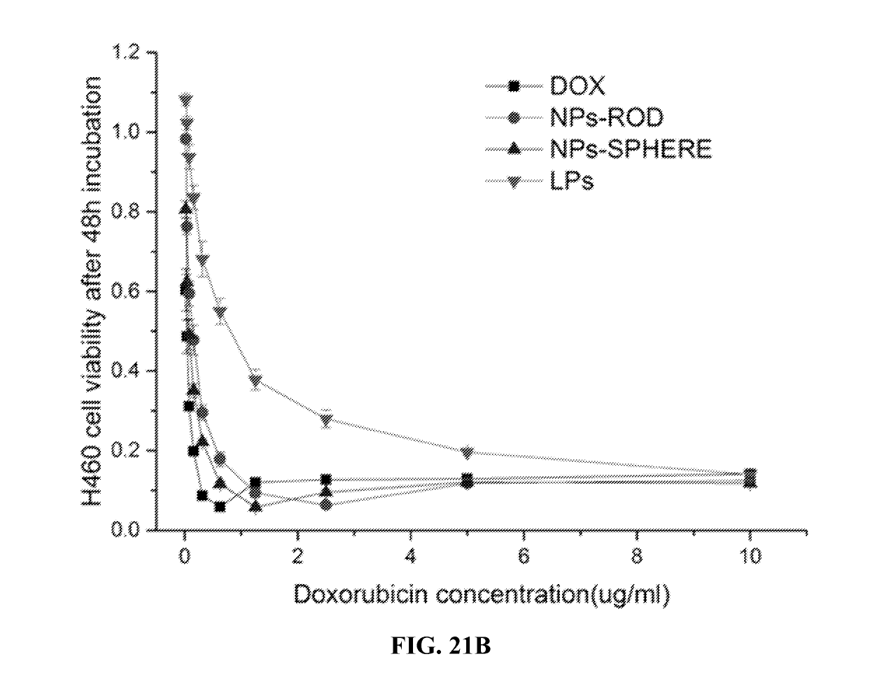

The synthesis procedure was as follows: (H-Cys-OMe)₂·2HC1 (10.0 mmol) and bis-fatty acid ester (10.0 mmol) were dissolved in 10.0 mL DMSO, then triethylamine (15 mmol) was added. The solution was stirred for 15 min. to obtain a uniform mixture, precipitated in 250 mL of cold ethyl ether, decanted, dried, re-dissolved in methanol, and re-precipitated in cold ethyl ether for further purification. The purification was repeated twice before drying in a vacuum at room temperature. The final product was a yellow or brown yellow solid powder. The chemical structure of polymer was confirmed by ¹H NMR. Exemplary ¹H and ¹³C NMR spectra and DSC plots are shown in FIGS. 2A to 2M. FIG. 2A is the ¹H NMR spectrum of cysteine dicarboxylate copolymer with sebacoyl chloride (Cys-OH-8) as the triethylamine salt in DMSO-d6. FIG. 2B is the ¹³C NMR spectrum of Cys-OH-8 as the triethylamine salt in DMSO-d6. FIG. 2C is the ¹H NMR spectrum of Cys-OH-8 as the free acid in DMSO-d6. FIG. 2D is the ¹³C NMR spectrum of Cys-OH-8 as the free acid in DMSO-d6. FIGS. 2E to 2I are typical DSC plots for the cysteine polymers. FIG. 2E is a DSC plot for Cys-OMe-2/Cys-2E polymer. FIG. 2F is a DSC plot for Cys-OMe-4/Cys-4E polymer. FIG. 2G is a DSC plot for Cys-OMe-6/Cys-6E polymer. FIG. 2H is a DSC plot for Cys-OMe-8/Cys-8E polymer. FIG. 21 is a DSC plot for Cys-OMe-10/Cys-10E polymer. FIG. 2J is the ¹H NMR spectrum of Cys-OMe-2/Cys-2E polymer in DMSO-d6. FIG. 2K is the ¹H NMR spectrum of Cys-OMe-4/Cys-4E polymer in DMSO-d6. FIG. 2L is the ¹H NMR spectrum of Cys-OMe-6/Cys-6E polymer in DMSO-d6. FIG. 2M is the ¹H NMR spectrum of Cys-OMe-8/Cys-8E polymer in DMSO-d6.

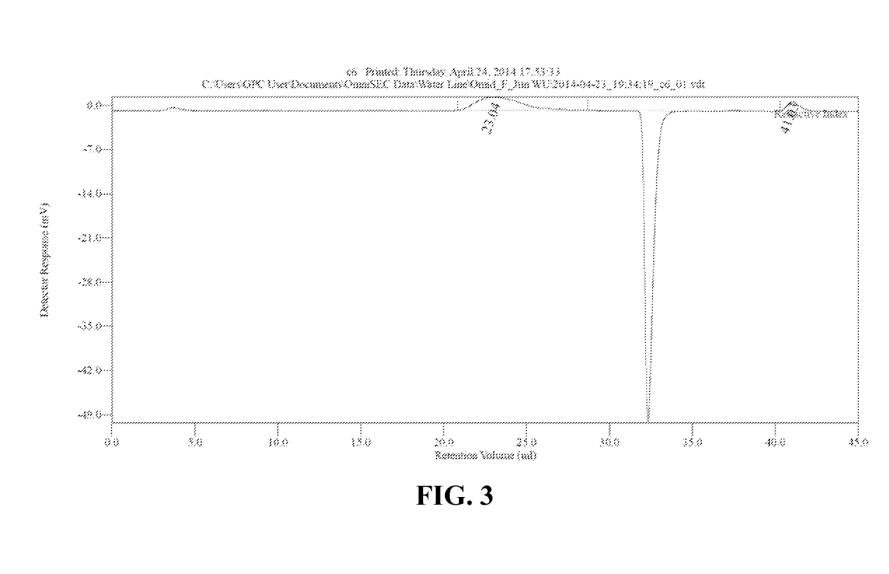

Polymer properties were characterized via GPC, DSC, elemental analysis, and solubility determinations. FIG. 3 is an exemplary chromatogram of a cysteine polymer made with these methods.

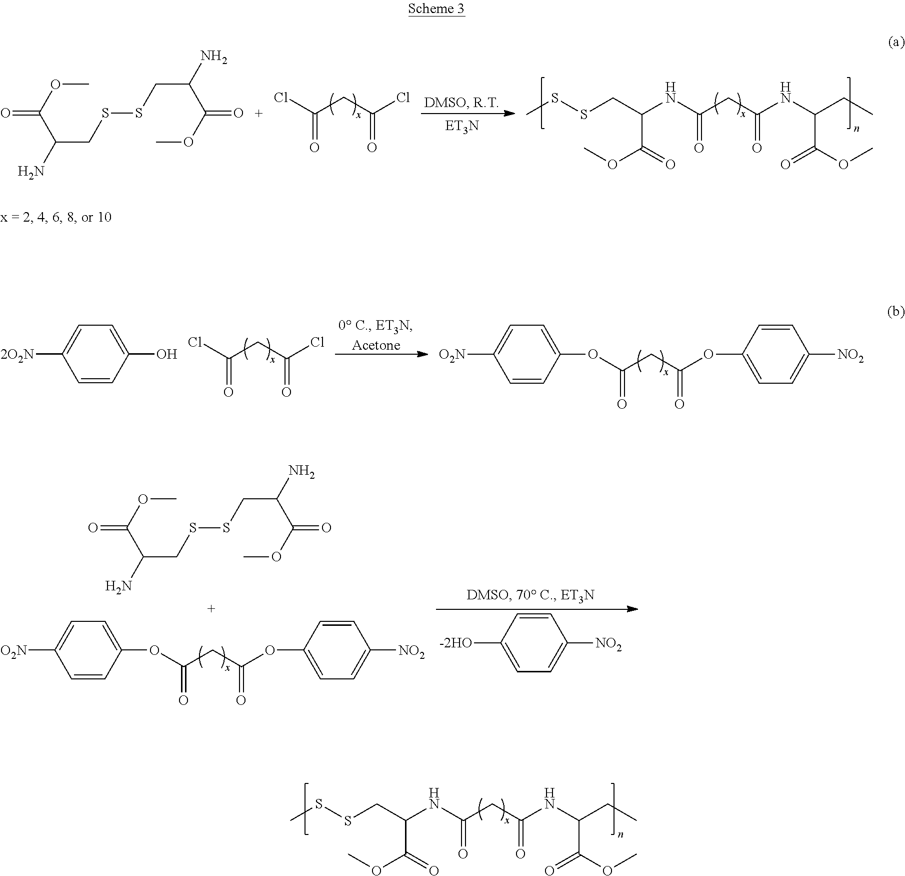

The preparation of linear hydrophobic L-cysteine-based poly disulfides involved the following synthesis parameters: 25° C. reaction temperature, 5-10 min. reaction time, DMSO as solvent, with the molar ratio of two monomers depending on the desired molecular weight and end functional groups. Chloroform or other suitable solvents can also be used. Triethylamine or other bases such as N,N-diisopropylethylamine can be used to catalyze the reaction. After reaction, the mixture was precipitated in ether, and the precipitated products were purified by repeating the precipitation three times. The final purified products were water-insoluble yellow powders, and the observed yields were >70%. The polymers were characterized by standard methods with yields, solubility, glass transition temperature (T_{g}), and molecular weight. The chemical structures of poly disulfide amides were confirmed by ¹H NMR.

Other strategies were also used to prepare the Cys-PDSA polymers such as diacid esters to react with cystine ester. An exemplary procedure is as follows. First, Di-p-nitrophenyl esters of dicarboxylic acids were prepared by reacting dicarboxylic acyl chloride varying in methylene length with p-nitrophenol as described by Wu et al., Advanced Functional Materials 2012, 22, 3815. Five esters were prepared: dip-Nitrophenyl Succinate (N2 with x=2); dip-Nitrophenyl Adipate (N4 with x=4); dip-Nitrophenyl Suberate (N6 with x=6); dip-Nitrophenyl Sebacate (N8 with x=8); di-p-Nitrophenyl Dodecanedionate (N10 with x=10). The “x” indicates the numbers of methylene groups in the diacid, and N10 is the first time reported. An example of the diacid monomer synthesis is given below. Di-p-nitrophenyl adipate (N4) was by the reaction of the adipoyl chloride (0.15 mol) with p-nitrophenol (0.31 mol) in acetone in the presence of triethylamine (0.32 mol). An ice/water bath was used to keep the p-nitrophenol and triethylamine mixed acetone solution (400 mL) at 0° C. Adipoyl chloride was diluted in 100 mL of cold acetone before being added dropwise into the above chilled solution with stirring for 2 h at 0° C. and overnight at room temperature. The resulting di-p-nitrophenyl adipate was precipitated in distilled water, washed completely, and then dried in vacuo at room temperature before final recrystallization in ethyl acetate. This purification process was performed three times. The final product is a brown-colored crystal.

Polymer solubility was determined at 1.0 mg/mL concentration at room temperature. Due to the hydrophobicity of cysteine ester and fatty diacid monomers, the Cys-OMe polymers were insoluble in buffers, distilled water, and in nonpolar organic solvents such as benzene, toluene, and ethyl ether; but soluble in more polar organic solvents such as DMSO, DMF, THF, methanol, acetonitrile, chloroform, and dichloromethane. The Cys-OH polymers were more soluble in water, but the polymer hydrophobicity still showed a dependence on the length of the diacid linker, as shown in Table 1.

TABLE 1
Solubility of Cys-OH triethylamine salt polymers in distilled water

| Polymer | Cys-OH-2 | Cys-OH-4 | Cys-OH-6 | Cys-OH-8 | Cys-OH-10 |
|---|---|---|---|---|---|
| Solubility | >200 | >200 | 100 | 5-10 | 5-10 |
| (mg/mL) | | | | | |

For many Cys-PDSAs, the melting point (Tm) was not identified. Cys-OMe-10/Cys-10E showed a Tₘ peak around 60° C. For other Cys-PDSAs (Cys-OMe-x/Cys-xE, with x from 2 to 8), the glass transition temperatures (T_{g}) were in the range of 0-45° C. An examination of the effect of the number of methylene groups in the diacid (x) part of the Cys-PDSAs revealed that an increase in x led to a lower T_{g} for x=2, 4, and 6. T_{g} decreased from 42 to 10° C. when the x value was increased from 2 to 6. For x=8, T_{g} was about 26° C.

The Mₙ of the Cys-OMe polymers were affected by synthesis conditions and could be adjusted accordingly. The typical Mₙ was between 3.0 kg·mol⁻¹ and 6.0 kg·mol⁻¹ with a polydispersity of 1.10-1.20. The x value did not have any significant impact on the Mn or polydispersity of Cys-PDSAs. Other properties, such as hydrophobicity, are also affected by the polymer structure. For hydrophobicity, an increase in x value enhanced it significantly, confirmed by the following NP characterization results.

In a similar manner, the cysteine carboxylic acid (Cys-OH) polymers were synthesized by the above method, and isolated as the triethylamine salt. The analysis of the Mn of the corresponding polymers are summarized in Table 2.

TABLE 2
Molecular weights of cysteine carboxylic
acid polymers, as triethylamine salts.

| Polymer | Cys-OH-2 | Cys-OH-4 | Cys-OH-6 | Cys-OH-8 | Cys-OH-10 |
|---|---|---|---|---|---|
| Molecular | 13,700 | 22,900 | 23,1300 | 18,300 | 18,200 |
| Weight | | | | | |
| (Mₙ, | | | | | |
| daltons) | | | | | |

The molar ratio of the monomers affects the molecular weight of the resulting polymer (Table 3).

TABLE 3
Effects of molar ratio of monomers on the size of
resulting polymer for Cys-OH-8 triethylamine salt.

| | 2:1 | 3:1 |
|---|---|---|
| Cys-OH-8 | (Cysteine:copolymer) | (Cysteine:copolymer) |
| Molecular Weight | 18,300 | 9,100 |
| (Mₙ, daltons) | | |

The cysteine polymers are sensitive to reductive conditions. Table 4 shows the effects of DTT treatment on Cys-OH-8 triethylamine salt after 4 hours. Increasing the concentration of DTT reduced the measured Mₙ of the polymer.

TABLE 4
Effects of DTT on Cys-OH-8 triethylamine
salt after 4 hour incubation.

| | Polymer | | | | |
|---|---|---|---|---|---|
| | | DTT/ | DTT/ | DTT/ | DTT/ |
| | Cys-OH-8 | Cys-OH-8 | Cys-OH-8 | Cys-OH-8 | Cys-OH-8 |
| | only | (0.1/1) | (0.5/1) | (1/1) | (10/1) |
| Molecular | 18,300 | 10,700 | 8,300 | 6,400 | 3,000 |
| Weight | | | | | |
| (Mₙ, daltons) | | | | | |

Table 5 shows the properties of various L-cysteine-based poly(disulfide amide) (Cys-PDSA) prepared by different combinations of (H-Cys-OMe)2.2HCl and dioyl chloride.

TABLE 5
L-cysteine-based poly(disulfide amide) (Cys-PDSA) prepared by
different combinations of (H-Cys-OMe)2.2HCl and dioyl chloride.

| | succinyl | adipoyl | suberoyl | sebacoyl | dodecanedioyl |
|---|---|---|---|---|---|
| | chloride | chloride | chloride | chloride | dichloride |
| | x = 2 | x = 4 | x = 6 | x = 8 | x = 10 |
| (H-Cys-OMe)₂•2HCl | Cys-2E | Cys-4E | Cys-6E | Cys-8E | Cys-10E |
| T_{g} or Tₘ (° C. ) | 44.15 | 31.92 | 19.80 | 27.72 | 58.92 |
| Mₙ (kg · mol⁻¹) | 12.1 | 17.2 | 16.4 | 18.5 | 20.9 |

### Example 2

### Preparation and Characterization of Cysteine Nanoparticles

### A. Cys-OMe Preparation

Cys-PDSA nanoparticles were prepared via the nanoprecipitation method. As an example, nanoparticles comprising cysteine dimethyl ester copolymer with sebacoyl chloride (x=8) (Cys-OMe-8 NPs) were prepared by first dissolving the polymer in DMSO to obtain a polymer solution with desired concentration (5.0-10.0 mg/mL). The polymer solution was then added dropwise to 20.0 volumes of distilled water and stirred with or without stabilizer, giving a final polymer concentration of 0.5-1.0 mg/mL. The following stabilizers were used: Pluronic® F-127, PVA, Tween® 80, MYRJ™, lipid, and lipid-PEG.

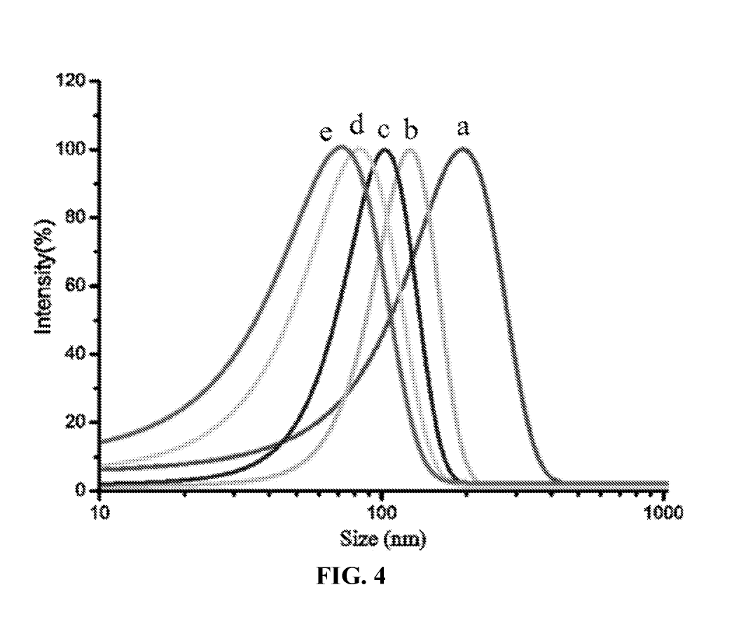

The particle size and distribution were measured by dynamic light scattering (DLS) at 25° C., with a scattering angle of 90°, and nanoparticle concentration of 0.1 to 0.5 mg/mL. The size of the nanoparticles was examined as a function of the x parameter (number of carbons in the alkyl linker) in deionized water at room temperature (FIG. 4). FIG. 4 is a plot of the size distribution of cysteine methyl ester nanoparticles as determined by DLS in deionized water at room temperature: y-axis is DLS % intensity; x-axis is nanoparticle size (nm). Curves: (a) Cys-OMe-2/Cys-2E NPs; (b) Cys-OMe-4/Cys-4E NPs; (c) Cys-OMe-6/Cys-6E NPs; (d) Cys-OMe-8/Cys-8E NPs; (e) Cys-OMe-10/Cys-10E NPs. DLS was used to compare the size of different nanoparticles. The results showed that the Cys-OMe nanoparticles have a median size around 100 nm, with the median size increasing as alkyl linker length of Cys-PDSAs decreased.

Transition electron microscopy (TEM) was used to confirm structure of the nanoparticles (FIGS. 5A-5S). A solution of nanoparticles in distilled water (0.1-0.5 mg/mL) was absorbed on a grid and negatively stained for 15 seconds. For each sample, 5-6 grids were prepared and viewed. Images were normally taken at 13-49,000× magnification. TEM with negative staining by uranyl acetate confirmed the median nanoparticle size and polydispersity determined by DLS. FIG. 5A shows an example of TEM images for the Cys-OMe-8/Cys-8E NPs. In some cases, TEM data indicated that the Cys-PDSA nanoparticles showed a spherical morphology, for example, as shown in FIGS. 5A-5J. In some cases, the nanoparticles showed a nanorod structure, as in FIGS. 5K-5M. Still others exhibited a nanofiber structure, as shown in FIG. 5N; a nanoring, as shown in FIG. 50; a bell-rod structure, as seen in FIG. 5R; or a nano round column, as shown in FIG. 5S.

The nanoparticle surface zeta potential was measured and recorded as the average of five measurements, using a nanoparticle concentration of approximately 0.1 to 0.5 mg/mL. The surface charges indicated that all the Cys-PDSA nanoparticles exhibited a negatively charged surface with the nanoparticle zeta potential in the range of −20˜−30 mV.

### B. Metal Ion Loaded Nanoparticles

A cysteine polymer stock solution (50 mg/mL) and a metal ion stock solution (25 mg/mL) were each prepared in DMSO. The polymer solution and metal ion solution were added to a centrifuge tube to result in the desired molar ratio, e.g., about 2:1, 3:1, 4:1, 5:1, 6:1, or 7:1, per binding site (assuming two binding sites per molecule polymer for Cys-OH polymer) followed by an appropriate amount of DMSO to give a desired concentration. In one example, 50 μL of polymer solution (2.5 mg) and an amount of metal ion solution was added to a centrifuge tube to result in a 6:1 ratio of polymer:metal ion. DMSO was added to give a total volume of 250 μL. The solution was mixed by vortexing for 5 s. Alternatively, the solutions could be mixed by sonication.

In a separate vial, 4.75 mL distilled water was stirred. The polymer-metal solution was added dropwise to the water slowly (2-3 s per drop) by pipette to give a final concentration of the polymer of about 0.5 mg/mL.

### C. Lipid Coating Protocol

Exemplary lipid coating protocols for nanorod, nanosphere, or nanoring structures are as follows. 14:0 TAP (1,2-dimyristoyl-3-trimethylammonium-propane (chloride salt) from Avanti Polar Lipids, Inc.), 18:0 PC (phosphatidylcholine, DSPC), DSPE-PEG3000, and cholesterol were used in 2:1:1:2 ratio by weight. Alternatively, 14:0 TAP and DSPE-PEG3000 (2:1 ratio) or 18:0 PC and DSPE-PEG3000 (1:1 ratio) were used.

The above lipid mixtures were made in ethanol to give a final concentration of 10 mg/mL. The resulting lipid solution was added to a stock mixture of nanoparticle (around 0.2-0.4 mg/mL) with stirring at 800-1000 rpm in a centrifuge. The mixture was stirred at 200-400 rpm for 2-4 h to evaporate the ethanol. PBS (10×) was then added to obtain the PBS nanoparticle solutions.

### D. Exemplary Protocols for Nanoparticle Fabrication

The following is an example of an protocol for preparing NPs of Cys-PDSA or PLGA in distilled water without stabilizer:

1. Dissolve 20.0 mg of Cys-PDSA or PLGA in 1.0 mL DMSO or DMF.

2. Precipitate the polymer solution dropwise to 19 volumes of stirring distilled water (1000 rpm) to obtain a Cys-PDSA or PLGA NP solution (1.0 mg/mL). DMSO or DMF could be removed by dialysis or centrifuge.

The following is one example of optimized protocol for preparing Cys-PDSA or PLGA NPs PBS solution with stabilizer:

1. Dissolve 20.0 mg of Cys-PDSA or PLGA in 1.0 DMSO or DMF.

2. Precipitate the polymer solution by adding dropwise to 19 volumes of stirring distilled water with stabilizer (such as DSPE-PEG3000, 20 wt % of NP) (1000 rpm) to obtain a Cys-PDSA/DSPE-PEG3000 or PLGA/DSPE-PEG3000 hybrid NP distilled water solution (1.0 mg/mL).

3. The resulting NP solution could be concentrated, and the remaining free molecules or organic solvent were removed by washing the NP solution three times using an Amicon Ultra-4 centrifugal filter (Millipore, Billerica, Mass.) with a molecular weight cutoff of 100,000 Da. Then concentrated PBS buffer was added to obtain a final 1× concentration of PBS.

The following is one example of optimized protocol for preparing Cys-PDSA NPs with 10 wt % docetaxel loaded PBS solution with stabilizer:

1. Dissolve 18.0 mg of Cys-PDSA and 2.0 mg docetaxel in 1.0 DMSO or DMF.

2. Precipitate the polymer drug mixture solution dropwise to 19 volumes of stirring distilled water with stabilizer (such as DSPE-PEG2000, 20 wt % of NP) (1000 rpm) to obtain a 10 wt % docetaxel Cys-PDSA/DSPE-PEG2000 hybrid NP distilled water solution (1.0 mg/mL).

3. The resulting NP solution could be concentrated, and the remaining free molecules or organic solvent were removed by washing the NP solution three times using an Amicon Ultra-4 centrifugal filter (Millipore, Billerica, Mass.) with a molecular weight cutoff of 100,000 Da. Then concentrated PBS buffer was added to obtain a final 1× concentration of PBS.

### Example 3

### Loading of Hydrophobic Small Molecule into Cysteine Nanoparticles

The drug or fluorescent-dye-loaded nanoparticles were prepared by mixing predetermined amounts of polymer and drug/dye in the DMSO, then following the nanoprecipitation procedure.

A cysteine polymer stock solution (50 mg/mL) and a small molecule stock solution (25 mg/mL) were each prepared in DMSO. The polymer solution and small molecule solution were added to a centrifuge tube to result in the desired molar ratio, e.g., about 2:1, 3:1, 4:1, 5:1, 6:1, or 7:1, followed by an appropriate amount of DMSO to give a desired concentration. In one example, 50 uL of polymer solution (2.5 mg) and an amount of small molecule solution was added to a centrifuge tube to result in a 6:1 ratio of polymer: small molecule. DMSO was added to give a total volume of 250 μL. The solution was mixed by vortexing for 5 s. Alternatively, the solutions could be mixed by sonication.

In a separate vial, 4.75 mL distilled water was stirred. The polymer-small molecule solution was added dropwise to the water slowly (2-3 s per drop) by pipette to give a final concentration of the polymer of about 0.5 mg/mL.

As a hydrophobic fluorescent probe, Nile red was used to compare the hydrophobicity of different nanoparticles. Photo images were also utilized to record the color or other appearance change of the nanoparticle solutions. Nile red (1 wt %), was loaded into Cys-OMe-x/Cys-xE nanoparticles during formulation. Nile red is a polarity-sensitive fluorescent probe and shows strong fluorescence in a hydrophobic environment but weak to no fluorescence in hydrophilic environments such as aqueous media. Thus Nile red-loaded nanoparticles would exhibit a different fluorescent intensity (color) compared to Nile red along according to the microenvironment. The difference in fluorescence intensity can be correlated with a hydrophobicity difference among the different Cys-OMe-x/Cys-xE nanoparticles. Fluorescence intensity increased with the x value (number of CH₂ groups) in the repeated linker unit, which indicated that the nanoparticle hydrophobicity increased with increasing x value. Nanoparticles with x values 4, 6, and 8 showed similar fluorescent intensity, and thus exhibited similar hydrophobicity under this evaluation. For Cys-OMe-2/Cys-2E (x=2) and Cys-OMe-10/Cys-10E (x=10) nanoparticles, the weak and strong fluorescent intensity correlated with low and high hydrophobicity, respectively.

### Example 4

### In vitro Stability of Nanoparticles

For the in vitro stability study, nanoparticles were incubated with a variety of solutions at 37° C. at a concentration of 0.5 mg/mL. At each time point, an aliquot of nanoparticle solution was collected to measure nanoparticle size using quasi-elastic laser light scattering. The following solutions were used for the stability determinations: distilled water, 1 x PBS buffer, pH 5 buffer, and 10% FBS DMEM media. The measurements were performed in triplicate at room temperature.

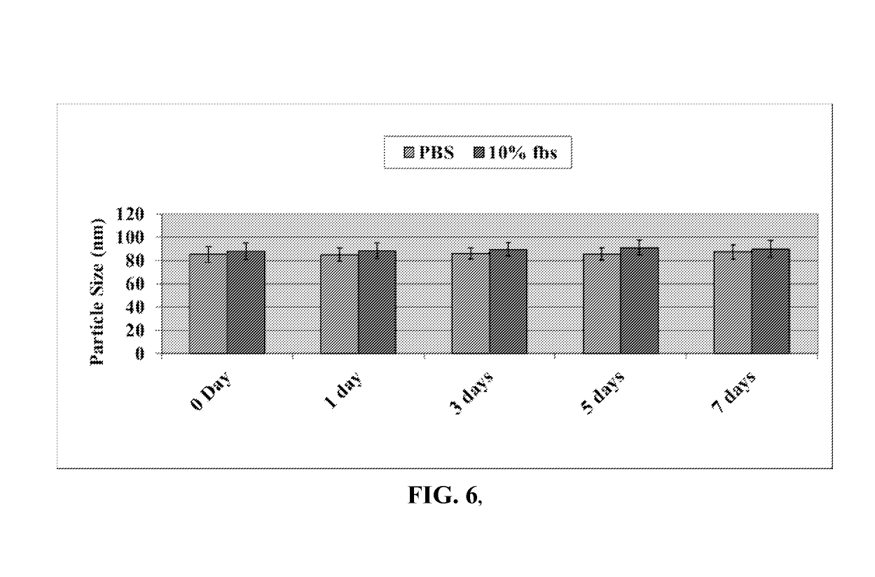

The stabilizers included PVA (MW), Tween® 80, F127, PEG, MYRJ™, lipid, and lipid-PEG. These nanoparticles were synthesized from Cys-PDSA and stabilizer via a single-step nanoprecipitation method: Cys-PDSA self-assembled and precipitated to form a hydrophobic core, while the stabilizer encapsulated the Cys-PDSA core to form a stabilizer monolayer of the nanoparticle. After incorporation of a stabilizer onto the nanoparticle surface, the stability of the resulting nanoparticles was evaluated in PBS buffer. DSPE-PEG (MW =2,000, 3,000 or 5,000 Da) at 20 wt % of nanoparticles were found to effectively stabilize the nanoparticles in PBS buffers. The surface charge of DSPE-PEG stabilized Cys-PDSA NPs was determined to be negative and in the range of -10--30 mV. The results are shown in FIG. 6, which plots particle size of Cys-8E/DSPE-PEG-3000 in 1× PBS buffer and 10% FBS DMEM cell culture medium over a period of one week. The results show that the particles remained stable in vitro.

### Example 5

### Behavior of Cys-PDSA Nanoparticles under Reduction Conditions

Redox-responsive performance of Cys-PDSA nanoparticles were evaluated under DTT reduction conditions. As the fluorescent indicator of hydrophobicity, Nile red was used to monitor Cys-PDSA nanoparticles. Nile red is an environment-sensitive fluorescent dye with very low water solubility, and demonstrates higher fluorescent intensity with increasing hydrophobicity of surrounding microenvironment. Nanoparticles were prepared with 1.0 wt % loaded Nile red. DTT stock solution was freshly prepared and added into different Cys-PDSA nanoparticle solutions to achieve a final concentration of 10.0 mM. The final nanoparticle concentration was fixed at 0.5 mg/mL. The fluorescence intensity changes of the nanoparticle solutions were monitored over a number of time points (up to 4 hours) at 37.0° C. by a Synergy HT Multi-Mode Microplate Reader (Ex=530 nm, Em=645 nm). Cys-PDSA nanoparticle PBS solutions without DTT were used as controls.

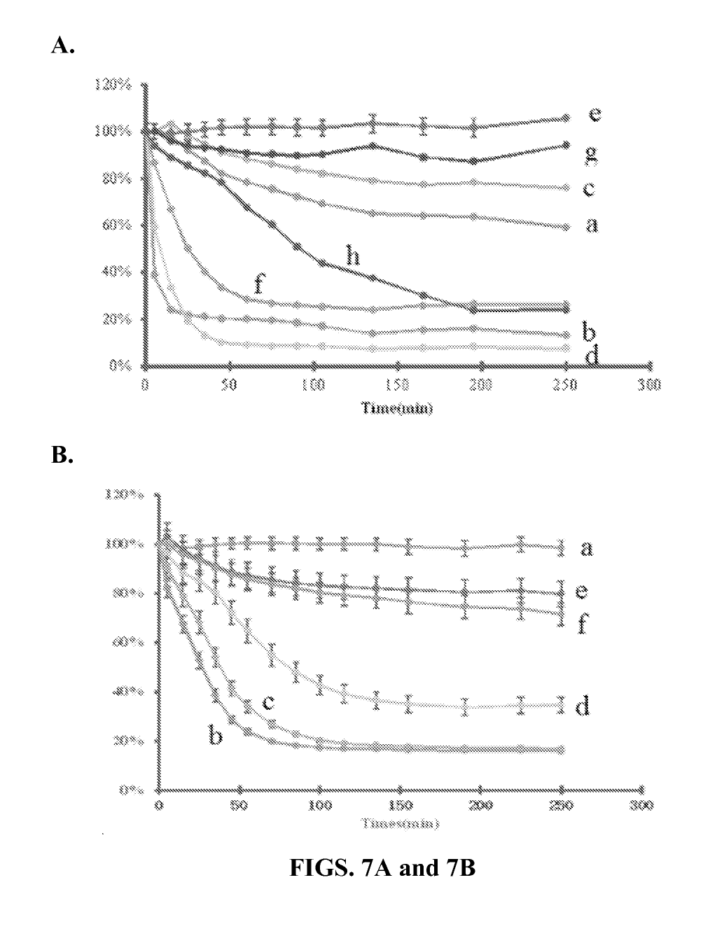

As shown in FIG. 7A, in a four-hour period, for the PBS group (without DTT), Cys-OMe-8/Cys-8E and Cys-OMe-10/Cys-10E nanoparticles did not show significant fluorescence intensity change. Meanwhile, the Cys-OMe-4/ Cys-4E and Cys-OMe-6/Cys-6E nanoparticles showed some decrease in fluorescence intensity, which means there was detectable Nile red released, since the nanoparticle itself was stable in PBS buffer. A 10.0 mM DTT PBS solution was used to mimic intracellular conditions. For the DTT group, the fluorescence intensity decreased for all the nanoparticles, and the rate decreased with increasing x. The fluorescence intensity of nanoparticles varied. For example, Cys-OMe-4/Cys-4E nanoparticles completely disassembled after 20 minutes, while Cys-OMe-6/Cys-6E and Cys-OMe-8/Cys-8E nanoparticles disassembled after about one hour. For the Cys-OMe-10/Cys-10E NPs, the fluorescence intensity decreased over about 3 h.

Reducing agent (DTT) concentration effect was evaluated at 0.1, 1.0, 5.0, and 10.0 mM DTT. Cys-OMe-8/Cys-8E nanoparticles in PBS were selected for this study, and 1.0 wt % Nile red was loaded into the Cys-PDSA nanoparticles as an indicator of disassembly rate. The final nanoparticle concentration was fixed at 0.5 mg/mL. Fluorescent intensity changes of the nanoparticle solutions were monitored using the aforementioned instrument for a number of time points (up to 4 hours) at 37.0° C. As shown in FIG. 7B, the NP disassembling rate increased with increasing DTT concentration.

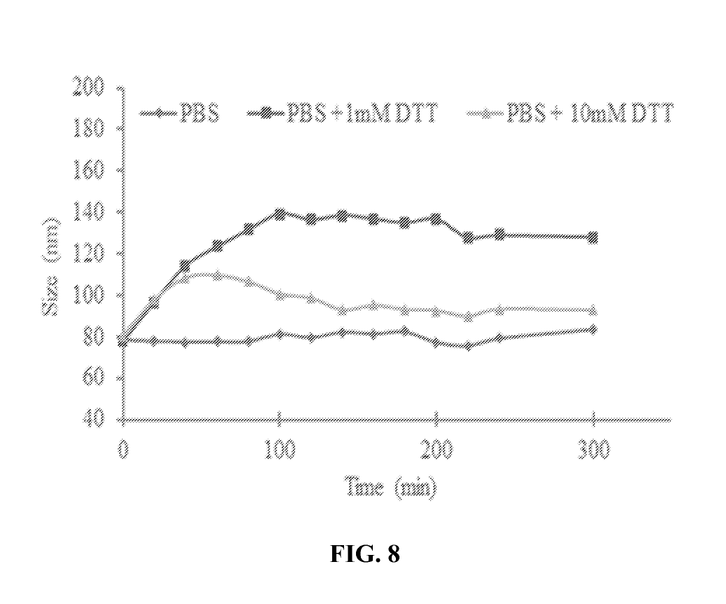

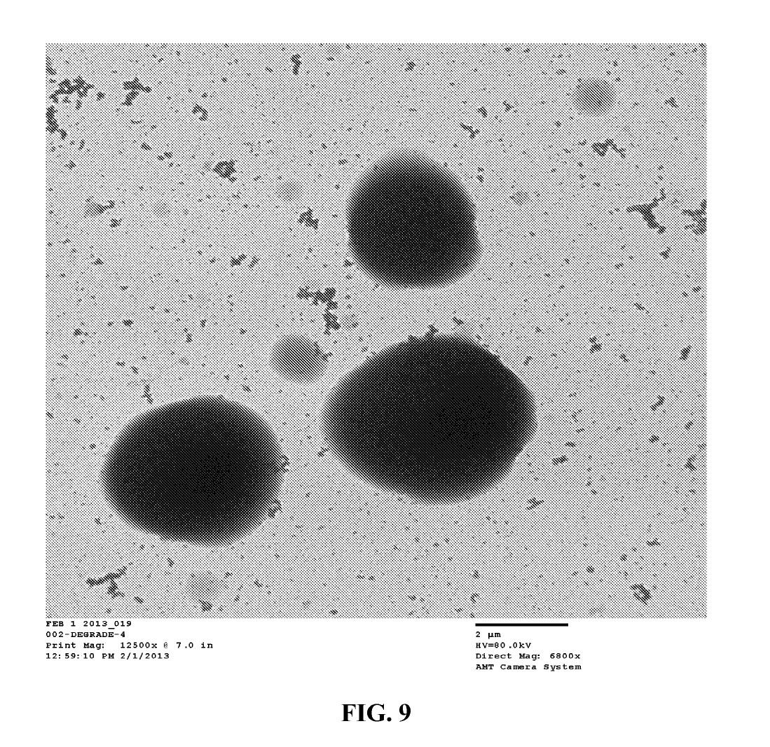

DLS and TEM were used to investigate the particle size and morphology changes of Cys-PDSA nanoparticles during redox-triggered disassembly over a number of time points at 37.0° C. FIG. 8 shows that the particle size as monitored by DLS increased slightly after the addition of DTT. Partial degradation under 1 mM DTT resulted in secondary assembly and large size change compared with the results under 10 mM DTT. TEM images (FIG. 9) indicated two types of structures were formed upon treatment with DTT: degraded, irregularly shaped NP debris from a few to tens of nanometers, and spherical structures ranging from hundreds to thousands of nanometers. This spherical structure was observed for the nanoparticles of x=8 and x=10. FIG. 10 further illustrates changes in morphology and hydrophobic diameter upon redox-triggered disassembly as monitored by TEM and shows images of dissembled Cys-8E NPs indicating second self-assembly.

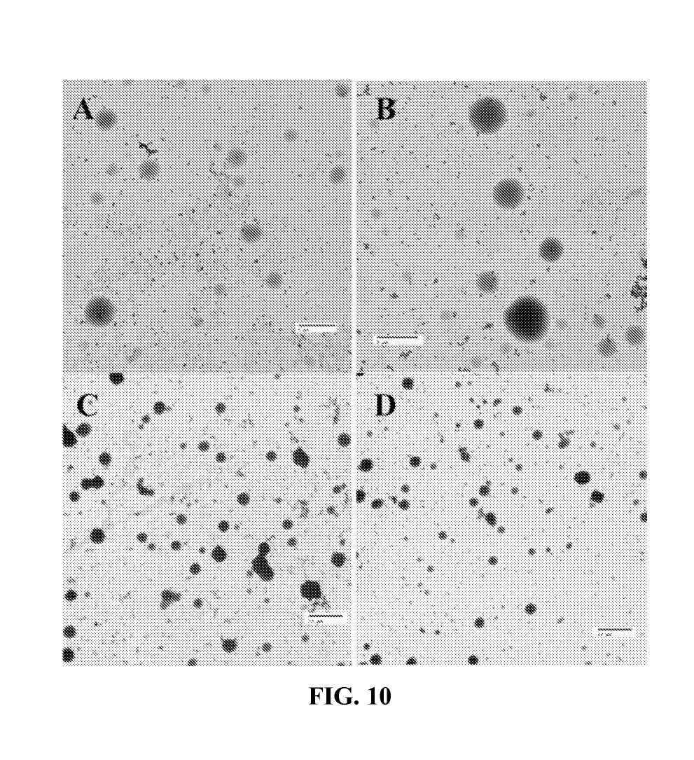

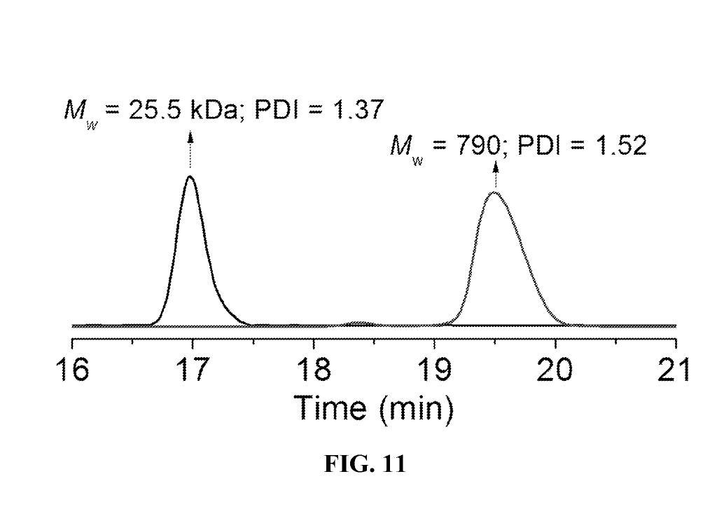

GPC tests of the MW changes of the reduced NPs also confirmed the significant reduction of the MW as shown in FIG. 11, which is a GPC profile of Cys-8E before and after 10 mM GSH treatment.

### Example 6

### In vitro Cytotoxicity of Nanoparticles

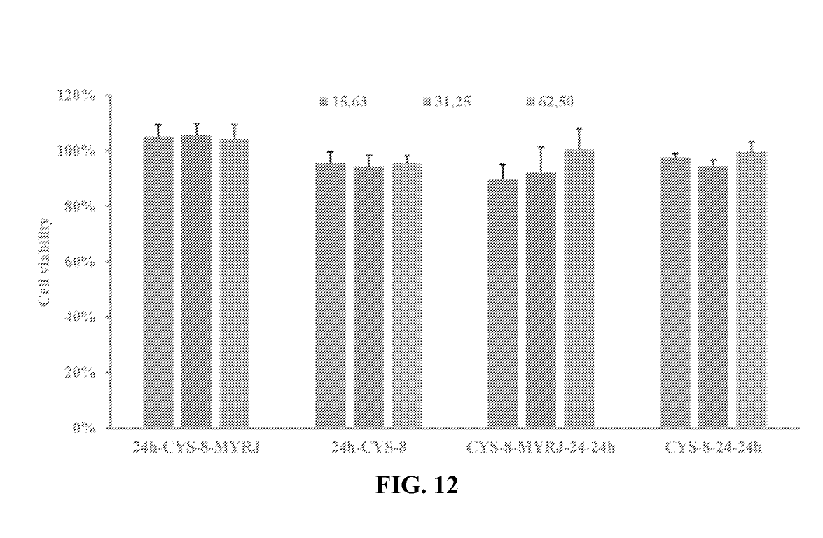

Polymer cytotoxicity was evaluated by Alamar blue assay. Nanoparticles were prepared and protected by 20% DSPE-PEG. Hela cells were used for 48 h tests. Cells (5,000 cells/well) were seeded in 96-well plates the day before use, and cultured according to recommendations from ATCC. 10% 0.1 mg/ml Alamar blue was first added into each well and the plate was incubated for another 2 h. Then the fluorescence intensity was measured by a microplate reader (excitation/emission=530 nm/590 nm). As shown in FIG. 12, which shows cell viability after treatment with various concentrations (15.63 μg/mL, 31.25 μg/mL and 62.5 μg/mL) of Cys-8E nanoparticles, the results indicated that the nanoparticles caused no significant cytotoxicity.

### Example 7

### FRET Effect of Nanoparticles

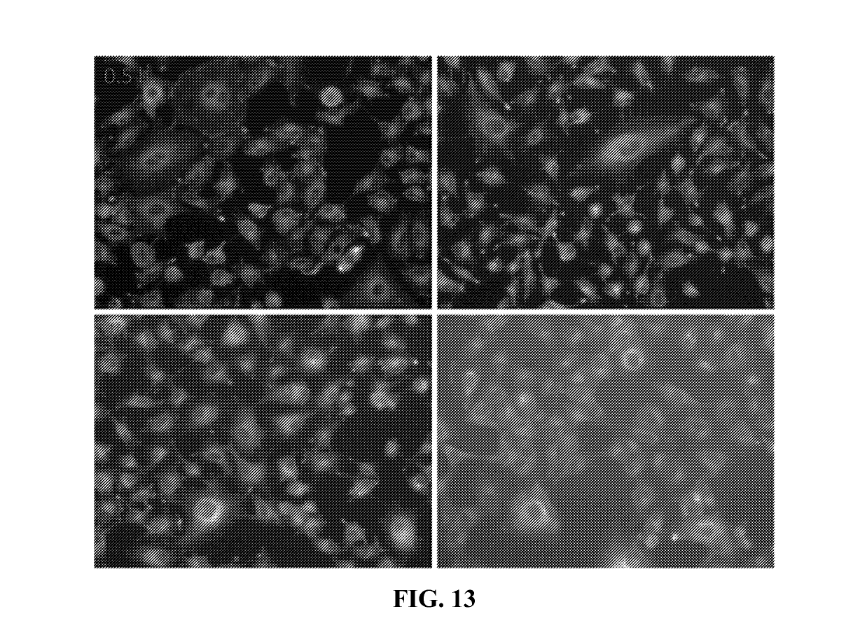

In order to confirm the unique redox sensitivity of the nanoparticles, FRET was used to investigate the redox-triggered mechanism inside cells (FIG. 13). Hydrophobic coumarin 6 and Nile red were selected as the donor-acceptor pair. The ratio of Nile red to coumarin 6 was 10:1. FIG. 13 shows that after 0.5 h treatment, cells showed red-green color under 400 nm excitation. Red showed the emission fluorescence of Nile red under FRET effect. After 1 h, a weak FRET effect was observed, and the green fluorescent intensity of coumarin 6 increased inside the cells. After 4 h, the cells showed a dominant green color, and the red color was significantly reduced. After 18 h, there was no apparent red color inside the cells.

### Example 8

### Loading and Drug Release of Cysteine Nanoparticles

### Example 8a

### Docetaxel

Docetaxel-loaded Cys-PDSA nanoparticles were prepared according to the standard nanoprecipitation procedure described in Example 3. Docetaxel loading ratios ranging from 1 wt % to 30 wt % were tested. After loading, the drug-loaded nanoparticle solution was washed three times to remove the residual DMSO and free drug. Nanoparticle solutions were then concentrated to give a Cys-PDSA concentration ≧10 mg/mL. DLS and TEM were used to investigate the particle size and morphology of drug-loaded Cys-PDSA nanoparticles.

HPLC methods were used to quantitatively measure the drug loading efficiency and release profile. The analytical conditions were: 232 nm monitoring, 1 mL/min. flow rate, 50/50 acetonitrile/water solvent. For in vitro drug release studies, concentrated nanoparticle PBS solutions were transferred into a 0.5 ml Spectrum dialysis kit (20000 MW cutoff). DTT PBS solution (10 mM) and blank PBS solution were freshly prepared. For each sample, six dialysis kits with nanoparticle solution loaded were dialyzed in 200 mL PBS at pH 7.4 under 37° C. At each predetermined time point, 50 μL aliquot of nanoparticle mixture was collected separately from three kits and mixed with an equal volume of acetonitrile to dissolve the nanoparticles. The Docetaxel concentration was determined by HPLC. Considering the nanoparticle stability, the maximun loading range of Cys-PDSA NPs are summarized in Table 6.

TABLE 6
Maximum Docetaxel-loading range of Cys-
PDSA at 0.5 mg/mL by nanoprocepitation

| Polymer | PLGA | Cys-2E | Cys-4E | Cys-6E | Cys-8E | Cys-10E |
|---|---|---|---|---|---|---|
| Maximum | ≦2% | ≦2% | ≦10% | ≦15% | ≦25% | ≦25% |
| Docetaxel | | | | | | |
| loading | | | | | | |

FIG. 5J shows examples of TEM images of Cys-OMe-8/ Cys-8E NPs loaded with 10 wt % docetaxel.

### Example 8b

### Other Drugs

The nanoparticles of the disclosure have also been loaded with other drugs, such as fingolimod as shown in the TEM image of FIG. 4B, and doxorubicin, as shown in the TEM images of FIGS. 5I and 5K to 5O. The doxorubicin examples showed that, in some cases, by modification of the polymer ionic state (i.e., free carboxylic acid or carboxylic acid salt), different shapes such as nanorods or nanospheres may be obtained.

### Example 8c

### Metal Ions

Additionally, the nanoparticles of the disclosure have been formulated in the presence of a variety of metal ion solutions, thus loading the nanoparticle with the metal ion. Examples are shown in FIGS. 5C-5H.

### Example 8d

### Load Release Dependence on pH

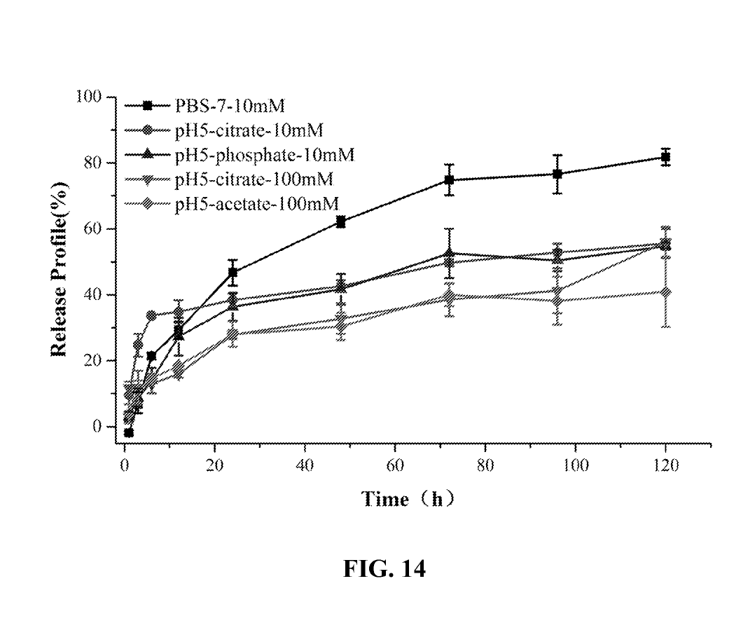

FIG. 14 is a plot showing the pH dependent release of doxorubicin from Cys-OH-8 triethylamine salt nanorod. After 120 h in PBS buffer at pH 7, about 80% of the drug was released from the nanoparticle. Other conditions at pH 5 buffers showed reduced drug release over the same time period.

### Example 8e

### Load Release Dependence on Detergent

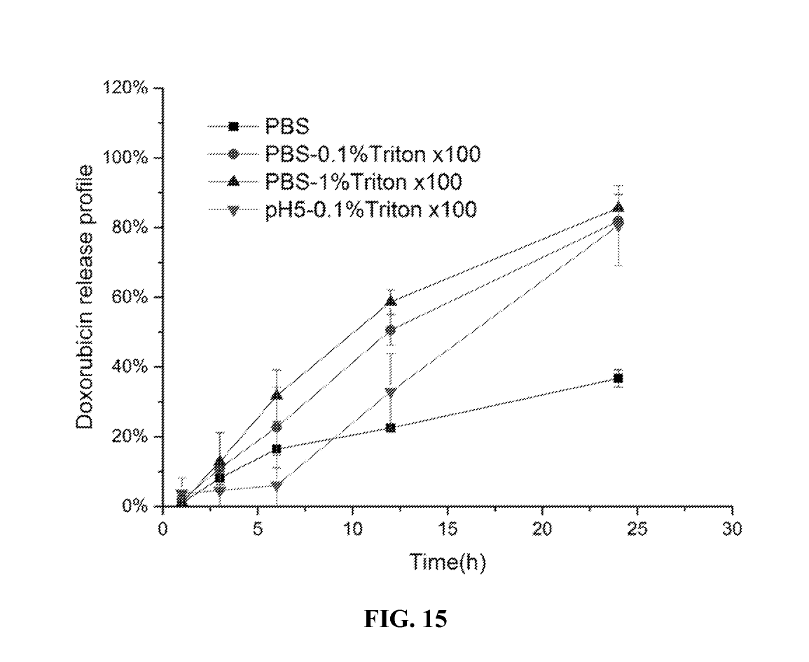

FIG. 15 is a plot doxorubicin release from Cys-OH-8 triethylamine salt nanorod as a function of pH and presence of TRITON® ×100 detergent, which enhanced the release of the drug over 24 h period.

### Example 8f

### Load Release Dependence on Reducing Conditions

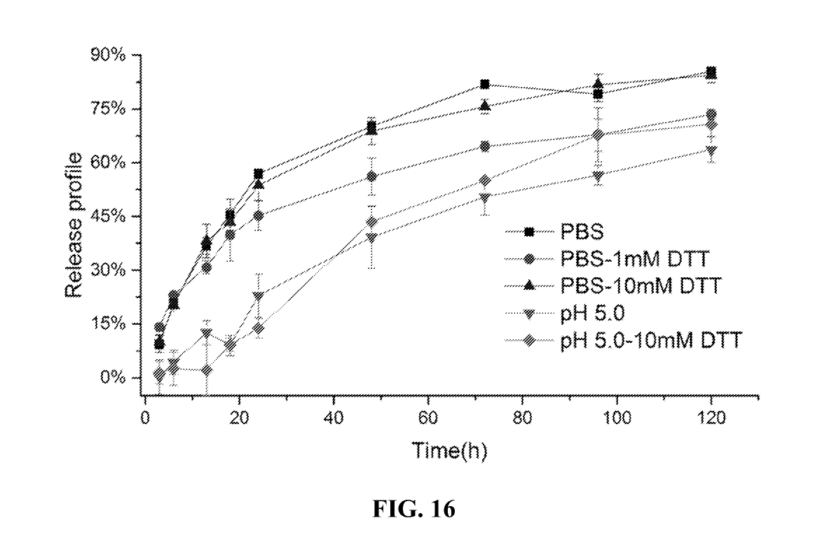

FIG. 16 shows doxorubicin release from Cys-OH-8 triethylamine salt nanorod as a function of DTT concentration.

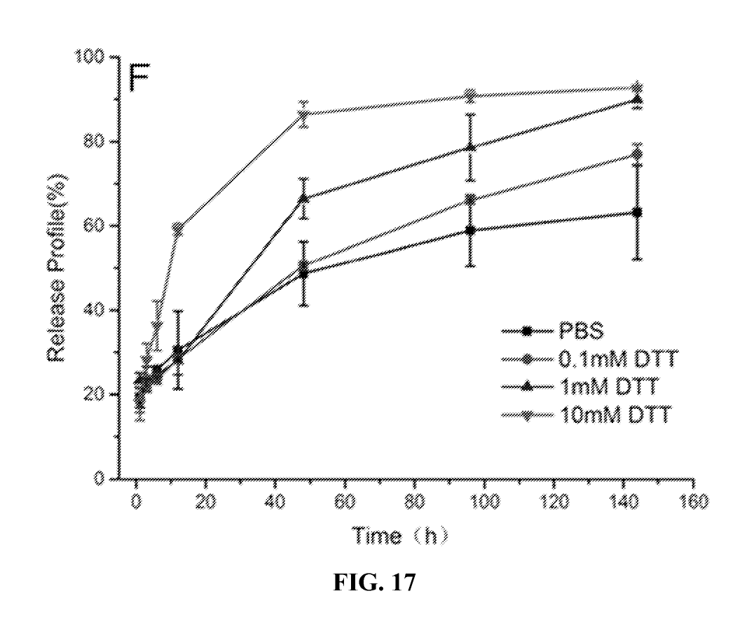

Docetaxel loading amount of Cys-OMe-8 (Cys-8E) NPs at 25 wt % of nanoparticles did not significantly affect the properties of the overall nanoparticle. DTT redox-triggered release was tested in 10 wt % docetaxel-loaded Cys-OMe-8 (Cys-8E) NPs as shown in FIG. 17, which shows the cumulative release of docetaxel from Cys-OMe-8 (Cys-8E) NPs. The results show that in PBS, the NPs release less than 60% of their drug content over 6 days. In contrast, upon addition of 1 or 10 mM DTT, the same quantity was released within 48 and 12 h, respectively. Compared with the PBS group, the 1 mM DTT and 10 mM groups had significantly different Dtxl release performance, confirmed redox-promoted discharge of payload. In 10 mM DTT pH 7.4 conditions, 90% docetaxel was released within 8 hours.

### Example 9

### Intracellular Degradation of Cys-PDSA NPs

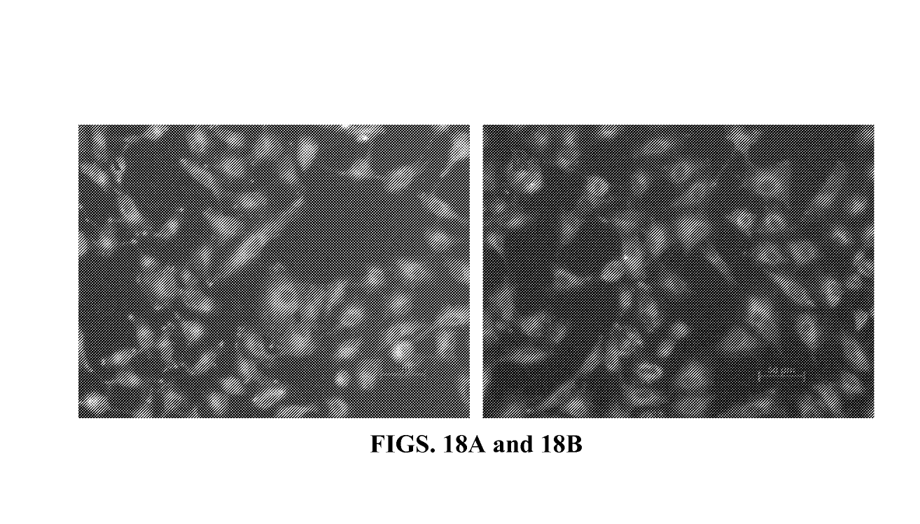

Fluorescence microcopy was employed to visualize the intracellular behavior of Cys-PDSA NPs. Nile red (Ex/Em=530/590nm) and coumarin 6 (Ex/Em=410/520nm) were co-encapsulated within the Cys-PDSA NPs as FRET (Fluorescence Resonance Energy Transfer) pair indicators to monitor the cellular uptake and structural changes of NPs. In this study, NPs loaded with 1.0 wt % fluorescent dyes (0.9% Nile red and 0.1% coumarin 6) were prepared, washed, and concentrated to remove free dyes. A549 cells were seeded into 12-well plates at a cell density of 3*10⁴ per well and pre-cultured for 24 h in 10% FBS F-12K medium. Prior to the internalization study, cells were washed with PBS buffer and incubated with Opti-MEM for 30 minutes before adding the NPs with a final concentration of 5.0 μg/mL (equivalent docetaxel) (N=3). At predetermined time points, cells were washed twice with PBS and imaged using a Zeiss fluorescence microscope (Ex=400 nm, Em=510 and 590 nm). The results are shown in FIG. 18A and 18B, which shows Fluorescence resonance energy transfer (FRET) effect for Cys-8E/DSPE-mPEG3000 hybrid NPs with 0.1 wt % Coumarin 6 and 1 wt % Nile Red. For FIG. 18A, A549 cells were used and pretreated with 50 μM nethylmaleimide (NEM) for 1 h to inhibit GSH and the mage was recorded at 4 h, while FIG. 18B shows an image recorded without NEM treatment with intracellular performance recorded after 4 h cell-uptake of NPs.

### Example 10

### Confocal Laser Scanning Microscopy of Nanoparticles (CLSM)

A549 cells were seeded in disc and incubated in 1 mL of F-12K medium containing 10% FBS for 24 h. Subsequently, the Dil-loaded NPs dispersed in F-12K medium (0.1 mg/mL) were added and the cells were allowed to incubate for another 1 h or 4 h. After removing the medium and subsequently washing with PBS (pH 7.4) solution several times, the endosomes and nuclei were stained with lysotracker green and Hoechst 33342, respectively. The cells were then viewed under CLSM (FV1000, Olympus, Japan) with DAPI channel for the nuclei and Alexa Fluor 488 channel for the endosomes. The results are shown in FIG. 19, which shows CLSM images of the A549 cells incubated with Cys-8E NPs for 1 h (A₁-A₄) and 4 h (B₁-B₄). The nuclei, endosomes, and NPs were stained using Hoechst 33342 (A₁-B₁), lysotracker green (A₂, B₂), and Dil (A₃, B₃), respectively.

### Example 11

### Cellular Effects of Doxorubicin Drug-loaded Cysteine

### Nanoparticles

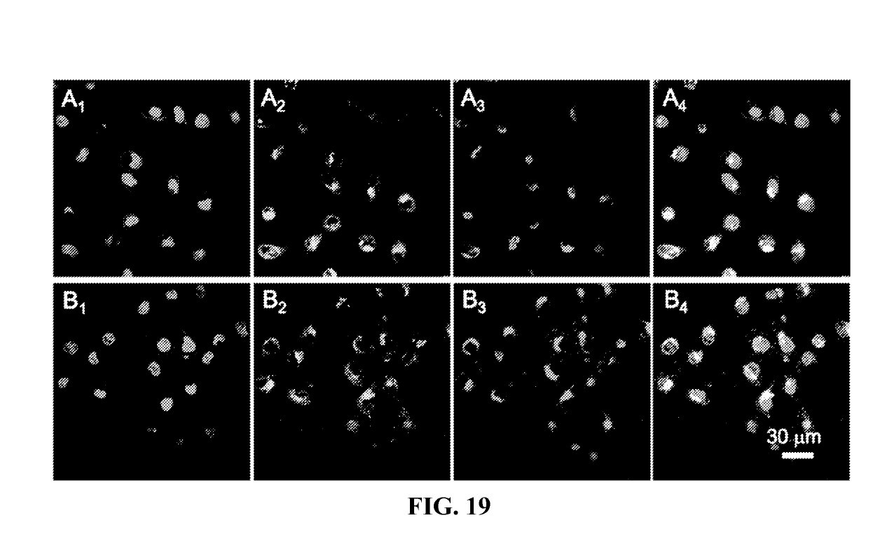

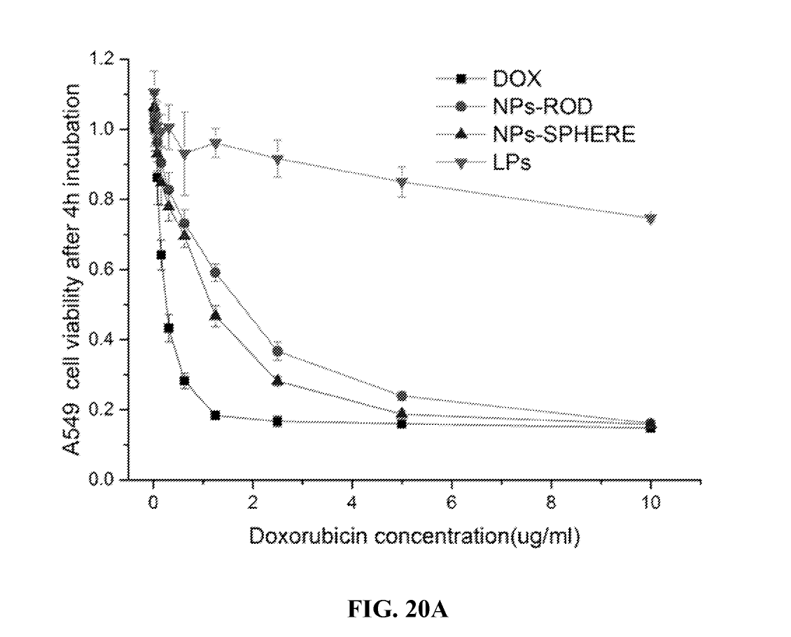

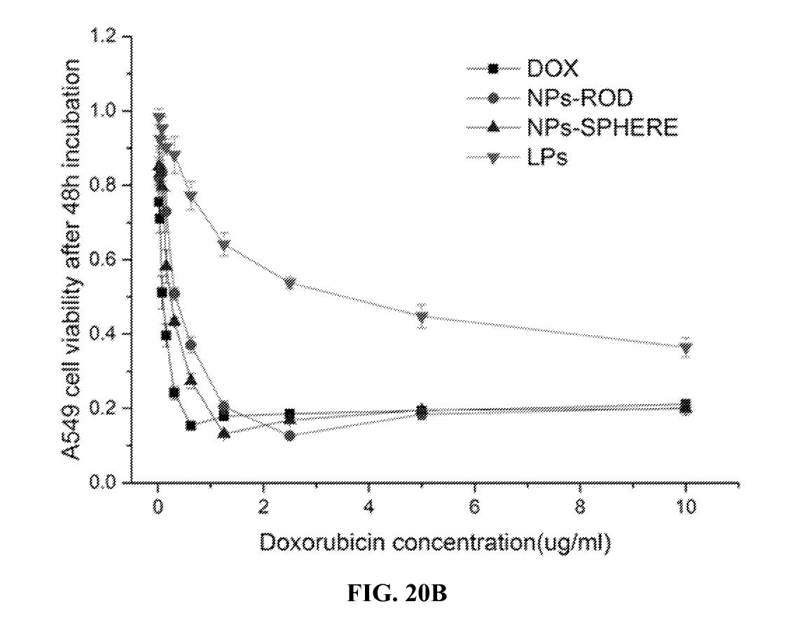

FIGS. 20A and 20B are plots showing the effects on cell viability of doxorubicin-loaded Cys-OH-8 triethylamine salt nanorods on A549 cells after 4 h incubation (FIG. 20A) and after 48 h incubation (FIG. 20B). FIGS. 21A and 21B show the effects on cell viability of doxorubicin-loaded Cys-OH-8 triethylamine salt nanorods on H460 cells after 4 h incubation (FIG. 21A) and after 48 h incubation (FIG. 21B).

Both doxorubicin-loaded nanorods and nanospheres showed anticancer properties against the cell lines tested.

### Example 12

### In Vivo Pharmacokinetics of Cysteine Nanoparticles

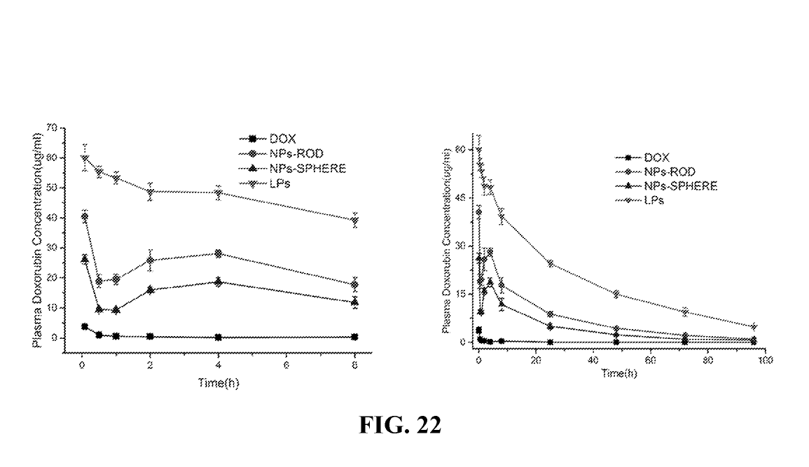

FIG. 22 is a pair of plots showing the in vivo pharmacokinetics of Cys-OH-8 NPs containing doxorubicin in A549 tumor mice for a single 5 mg/kg iv dose (y-axis: plasma doxorubicin concentration; x-axis: time). Both nanorods and nanospheres show a higher concentration of the drug in vivo than if the drug alone was intravenously dosed.

### Example 13

### Biodistribution of Cysteine Nanoparticles

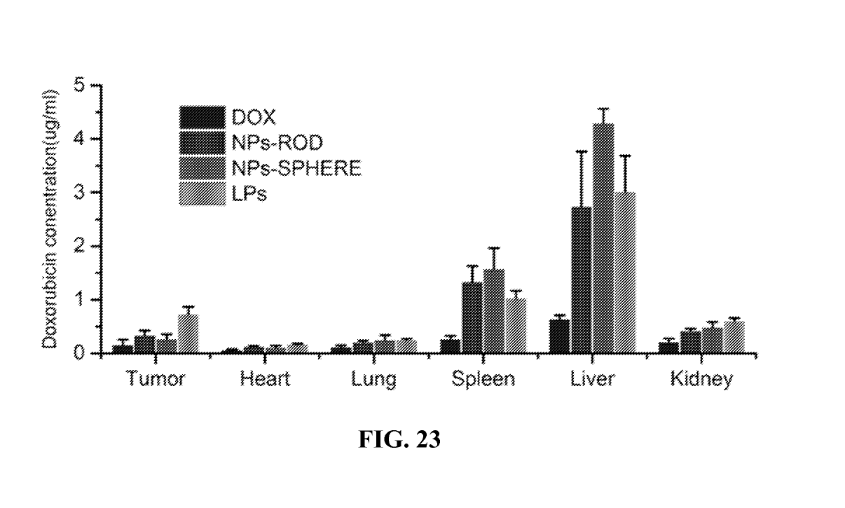

FIG. 23 is a plot showing the in vivo biodistribution data for Cys-OH-8 NPs containing doxorubicin in A549 tumor bearing mice for a single 5 mg/kg iv dose 48 h post-injection. Both nanorods and nanospheres showed higher concentration of doxorubicin in spleen and liver compared to when doxorubicin alone was dosed.

Cys-OMe-8/Cys-xE NPs loaded with docetaxel was also analyzed. To determine nanoparticle biodistribution, 0.1% DiR dye (Ex/Em: 748/780 nm) was loaded into Cys-PDSA docetaxel nanopaticles during nanoprecipitation. Nanoparticle solution was washed and concentrated to remove free drug or dye. DiR-labeling NPs (10 mg/kg equivalent docetaxel) was injected via the tail vein of tumor-bearing A549 xenograft bearing mice (20 days post inoculation). At 2, 24 and 48 hours after administration, mice were respectively euthanized and major organs were collected. Ex vivo NIR imaging was performed using an IVIS small animal in vivo imaging system (Caliper Life sciences).710ex/760em filter set was used as recommendation. All tissues were kept in −80° C. before test. Untreated control tissues were similarly obtained to correct for autofluorescence. All intensity were quantified by ImageJ software.

### Example 14

### Evaluation of Polymer Cytotoxicity and Anticancer Performance of Docetaxel-Loaded Cys-PDSA NPs

Hela, MCF-7, A549, and DU145 cells were used to evaluate in vitro anticancer performance of docetaxel-loaded NPs (Cys-OMe-8/Cys-8E NPs with 10 wt % docetaxel loaded with docetaxel concentration ranging from 1 ng/ml to 500 ng/mL). Hela, MCF-7, A549, and DU145 cells were used. Cells (5,000 cells/well) were seeded in 96-well plates the day before use, and cultured according to recommendations from ATCC. Cells were treated with different concentrations of free docetaxel or docetaxel loaded NP for 4 h or 48 h (N=5). For the 4 h experiment, treated cells were washed twice with PBS buffer and the plate was incubated for another 44 h after replacement of fresh medium. Cell viability at 48 h was determined by alamar blue assay. As a control, the polymer cytotoxicity on Hela cells was ascertained using empty NPs.

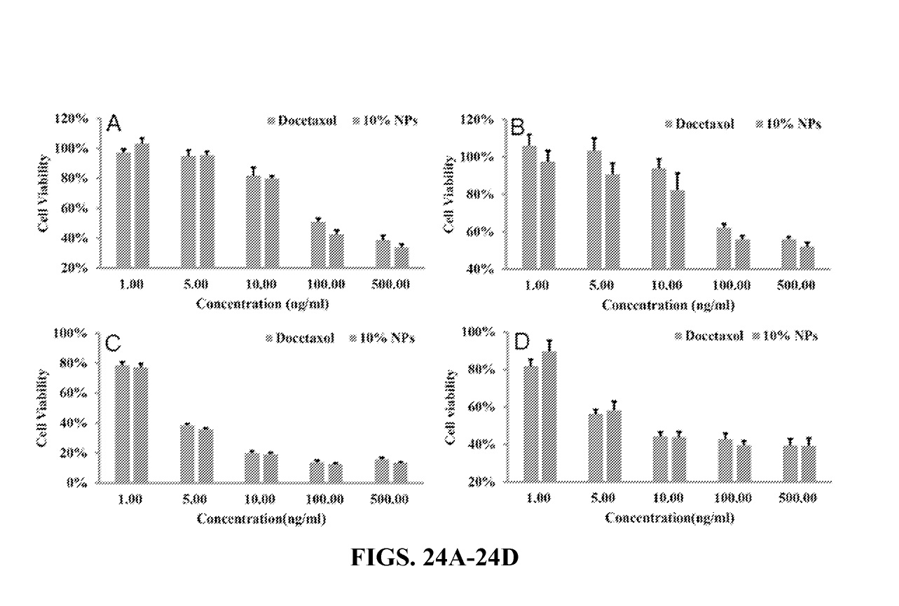

FIGS. 24A to 24D are plots showing the anticancer effects of docetaxel-loaded Cys-OMe-8/Cys-8E NPs in Hela and DU145 cells: In FIGS. 24A and 24C: HeLa cells were treated with docetaxel-loaded NPs for 4 h and 48 h, respectively. In FIGS. 24B and 24D: DU145 cells were treated with docetaxel-loaded NPs for 4 h and 48 h, respectively. At each concentration point, the bar on the left hand side shows cell viability for cells treated with free docetaxel (used as controls) while the bar on the right hand side shows cell viability for cells treated with docetaxel-loaded nanoparticles. Overall, drug-loaded NPs had anticancer effects similar to free docetaxel at similar concentrations without significant difference, though therapeutic efficacy varied among cell lines. With extension of the treatment period from 4 h to 48 h, both free drug and NPs showed dramatic improvement in inhibition of cell growth. For 4 h treatment, the IC₅₀ was about 100 ng/mL; at 48 h treatment, the IC₅₀ was about 5 ng/mL.

The Alamar blue assay was used to evaluate the NP cytotoxicity. Briefly, 10% 0.1 mg/mL alamar blue was first added into each well and the plate was incubated for another 2 h. Then the fluorescence intensity was measured by a microplate reader (excitation/emission=530 nm/590 nm).

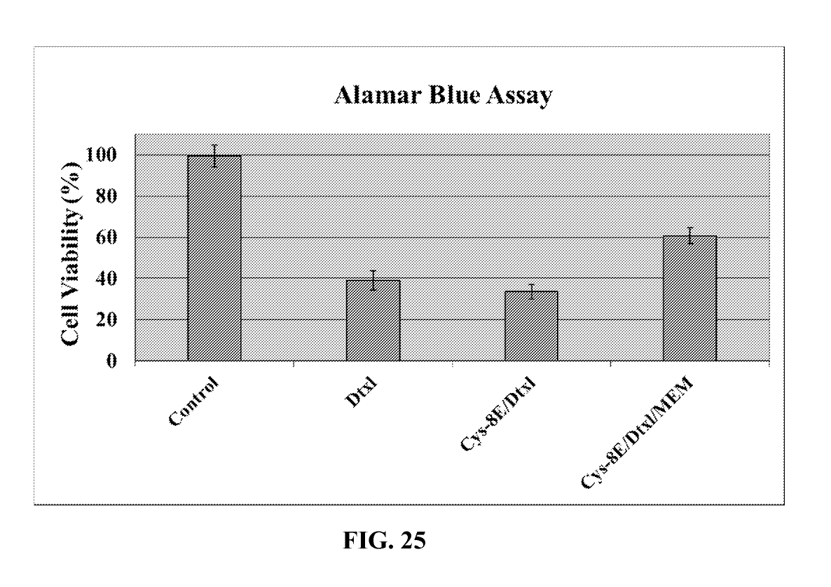

To investigate the effect of intracellular GSH on the anti-cancer activity of docetaxel-loaded Cys-OMe-8/Cys-8E NPs, HeLa cells were pretreated with 50 μM N-ethylmaleimide (NEM) for 1 h to inhibit cellular GSH, with other procedures being carried out as described above. The results are shown in FIG. 25, which is a plot of cell viability after treatment for 4 h with medium (control), free docetaxel (Dtxl), docetaxel-loaded Cys-8E NPs, and docetaxel loaded Cys-8E NPs and NEM (docetaxel concentration: 500 ng/mL). The results show that when HeLa cells were pre-treated with 50 μM NEM for 1 h, GSH depletion inhibited intracellular redox-sensitivity and reduced the anticancer performance of docetaxel-loaded Cys-8E NPs.

### Example 15

### Antitumor Effects of Drug-loaded Cysteine Nanoparticles

The antitumor efficacy was evaluated in the A549 Human Lung Carcinoma Xenograft model. All animal experiments were approved by Harvard Medical School.

### A. Doxorubicin

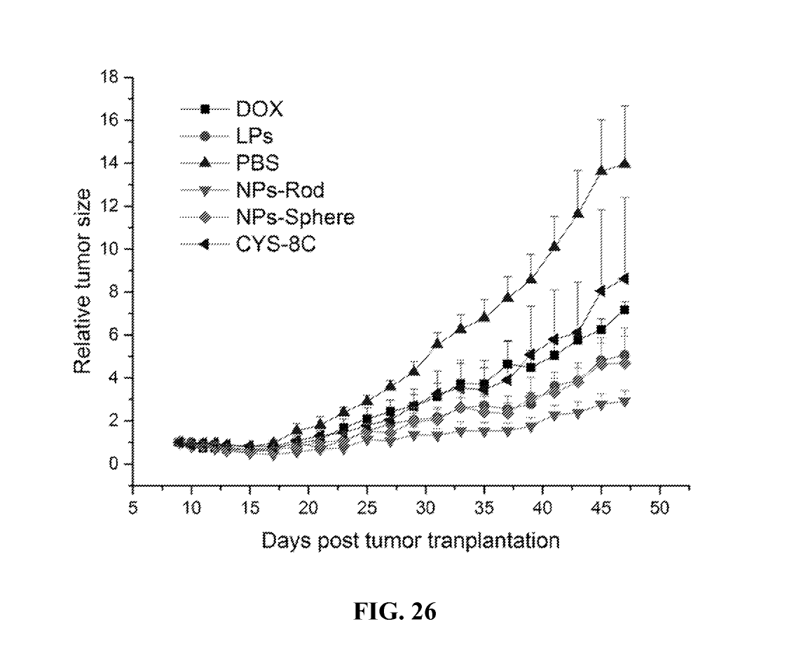

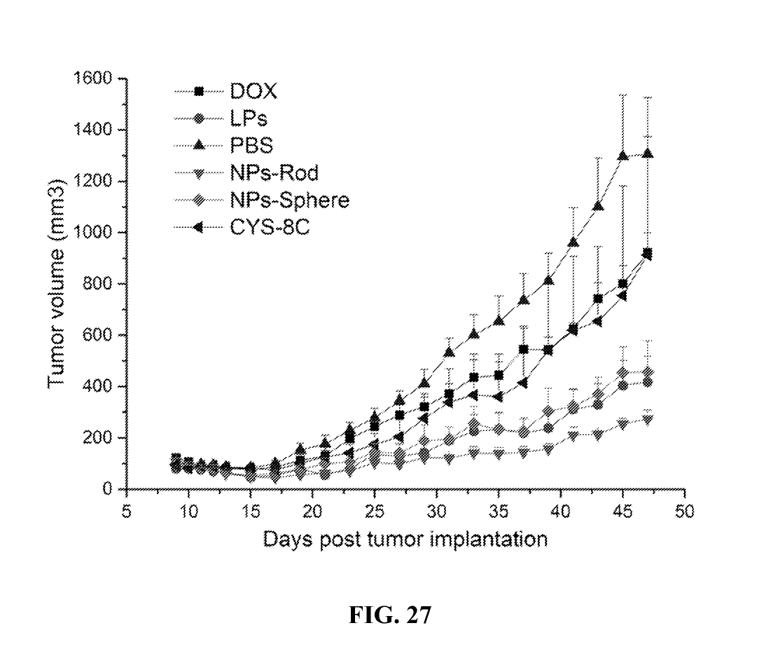

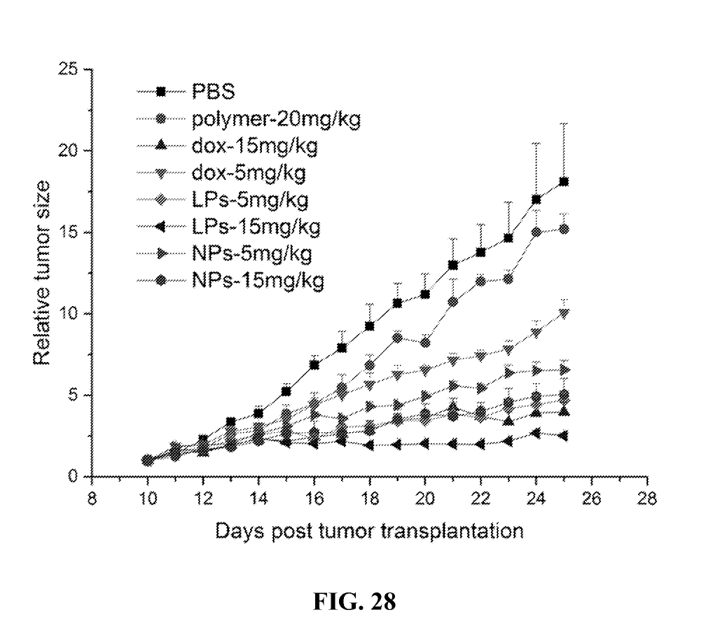

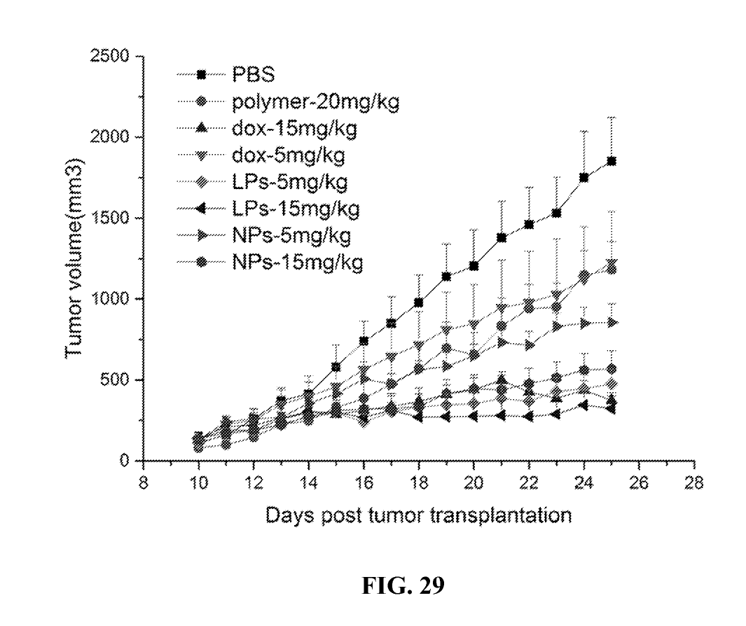

FIGS. 26-29 are plots showing the antitumor effects of doxorubicin-loaded Cys-OH-8 nanorods and nanospheres in A549 tumor bearing mice. Both doxorubicin-loaded nanorods and nanospheres had a greater effect on relative tumor volume and relative tumor size compared with doxorubicin mice alone at 5 mg/kg dose.

### B. Docetaxel

Antitumor efficacy was evaluated in the A549 Human Lung Carcinoma Xenograft model. A549 cell suspension (100 μL, equivalent to 5×10⁶ cells) was first mixed with 100 μL Matrigel (BD scientific Co.), then subcutaneously injected into the right flank of 5-week-old female athymic nude mice (Charles River). The treatment was initiated on the 9^{th} day, when solid tumor reached ˜100 mm³. Tumor-bearing mice were divided into 4 groups and injected daily for two weeks on the 9^{th} and 23^{rd} days after tumor inoculation with: (i) 1× PBS (ii) docetaxel 5 mg/kg (MTD), (iii) Cys-OMe-8/Cys-8E NPs at a docetaxel equivalent dose of 5 mg/kg (MTD), (iv) Cys-OMe-8/Cys-8E NPs at a docetaxel equivalent dose of 10 mg/kg. Control or drugs were administered by intravenous injection in the lateral tail vein (10 μL per g of body weight). Tumor dimension and body weight were recorded every other day. Tumor volume was calculated as follows: Volume [mm³]=length×width×0.5. Mice were regularly monitored (every other day) for physical performance and were euthanized upon exceeding 15% weight loss, over 1500 mm³ volume or if tumor ulceration was observed. Prior to the experiment, the maximum tolerated dose (MTD) of free docetaxel was established in normal Balb/c mice at 5 mg/kg. Tumor volumes at Day 30 were compared using the ANOVA analysis.

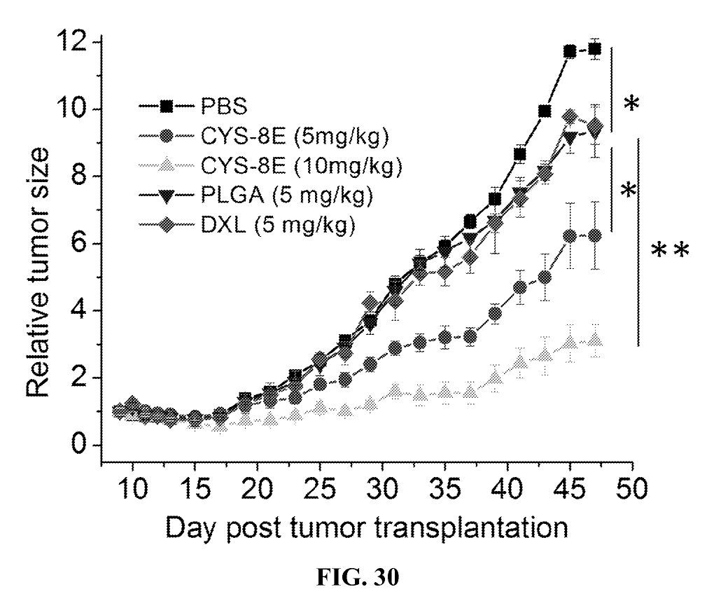

FIG. 30 is a plot of the antitumor effects of docetaxel-loaded Cys-OMe-8/Cys-8E nanoparticles in A549 tumor bearing mice. Free 5 mg/kg docetaxel, 5 mg/kg Cys-OMe-8 NPs, 10 mg/kg Cys-OMe-8/Cys-8E NPs and PBS saline solution were injected via the lateral tail vein (twice, on day 10 and 24 after tumor inoculation). The PBS control group exhibited rapid tumor growth, while free docetaxel has limited efficacy for tumor inhibition. In comparison, the docetaxel-loaded nanoparticles were able to suppress tumor growth. After 40 days (2 weeks after last administration), all groups demonstrated obvious tumor regrowth, but tumor growth rates were still varied. Nanoparticle treatment still resulted in smaller relative tumor size. In order to continually keep tumor inhibition, repeated dosage is necessary for long term treatment. In addition, all groups had similar growth trend of body weight, which indicated low nonspecific toxicity of the nanoparticles on treated mice. For comparison, results for a group of animals treated with PLGA NPs equivalent to 5 mg/kg of docetaxel are also shown. The PBS control group exhibited rapid tumor growth, while free docetaxel and docetaxel-loaded PLGA NPs showed only limited tumor inhibition. In comparison, 5 mg/kg of docetaxel loaded in NPs suppressed tumor growth for longer. For comparison purposes, the volume of tumors in mice injected took 35 days to reach that volume in mice treated with 5 mg/kg of docetaxel loaded in Cys-8E NPs.

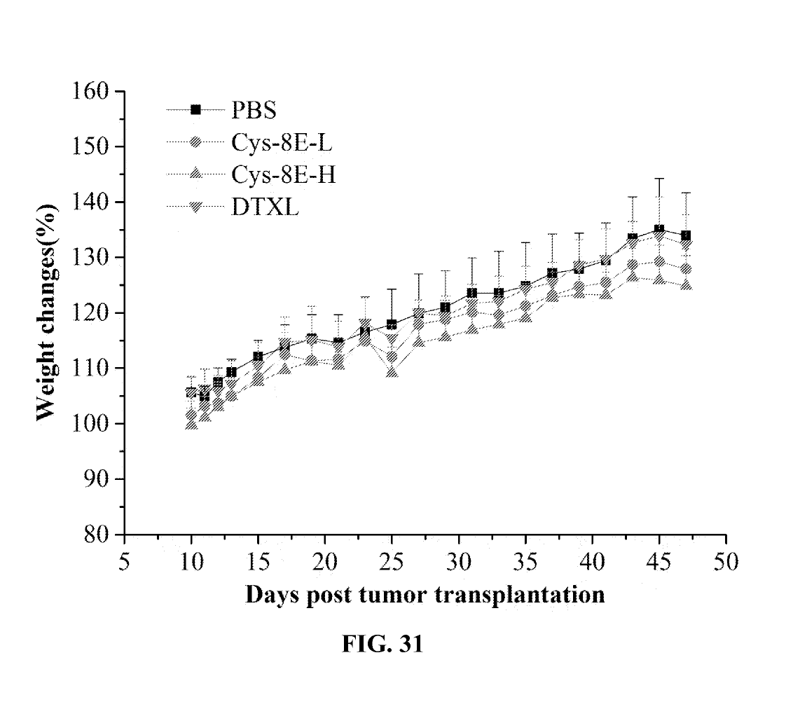

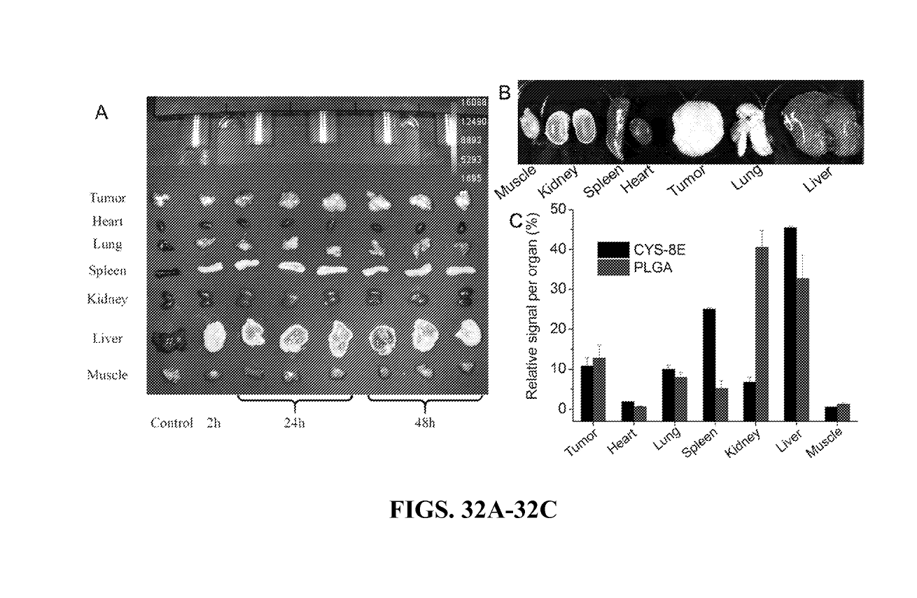

FIG. 31 shows the effect on mouse weight after treatment with control (PBS), (ii) docetaxel at 5 mg/kg, (iii) Cys-OMe-8/Cys-8E NPs at a docetaxel equivalent dose of 5 mg/kg (“Cys-8E-L”), (iv) Cys-OMe-8/Cys-8E NPs at a docetaxel equivalent dose of 10 mg/kg (“Cys-8E-H”).

The encapsulation of docetaxel in Cys-OMe-8/Cys-8E NPs allowed increasing the dose above the 5 mg/kg MTD without significant weight loss in mice (FIG. 31). The more-aggressive treatment (10 mg/kg) significantly improved tumor inhibition, with tumors not reaching 300 mm³ volume before day 48. Although tumor growth was observed in all groups, possibly because the study was designed with only two therapeutic doses, Cys-OMe-8/Cys-8E greatly reduced the size of tumors, confirming the therapeutic usefulness of redox-responsive polymeric NPs.

### Example 16

### In Vivo Biodistribution of Drug-loaded Cysteine Nanoparticles

The A549 xenograft model was also used to study the biodistribution of NPs loaded with docetaxel and 0.1% DiR dye (Ex/Em: 748/780 nm) at 2, 24, and 48 hours after administration. Fluorescent NPs were injected via the tail vein at 20 days post inoculation at a dose equivalent to 10 mg/kg of docetaxel. After euthanasia, primary organs were collected and kept at −80° C. until further use. Ex vivo NIR imaging was performed using an IVIS small-animal in vivo imaging system (Caliper Life Sciences) using 710/760 nm filters. Untreated control tissues were similarly obtained to correct for auto-fluorescence. Intensity was quantified using ImageJ software. The results are shown in FIG. 32A and 32B. FIG. 32A shows fluorescent images of the tumors and main organs of A549 tumor-bearing nude mice sacrificed at 2 h, 24 h, or 48 h post-injection of the NPs made with Cys-8E. FIG. 32B shows fluorescent images of the tumors and main organs of A549 tumor-bearing nude mice sacrificed at 24 h post-injection of the NPs made with PLGA. FIG. 32C provides quantitative biodistribution profiles of NPs in the tumors and main organs of A549 tumor-bearing nude mice sacrificed at 24 h post-injection of the NPs made with Cys-8E or PLGA. The results show that Cys-8E NPs and PLGA NPs show different biodistributions for major organs. Tumoral levels of Cys-8E NPs and PLGA NPs were similar, but they were only a very small portion of total major organ distribution (less than 10%) and may not be affected by the distribution in other organs.

### Example 17

### In Vivo Pharmacokinetics (PK) Study of Drug-loaded Cysteine Nanoparticles

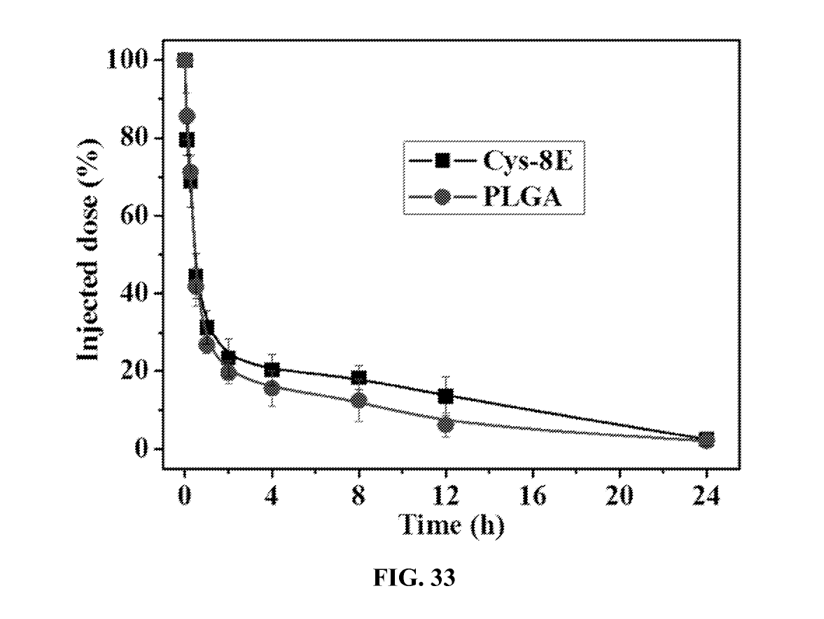

For an in vivo pharmacokinetic study, normal BALB/c mice (6-8 weeks old, n=5 per group) were injected with docetaxel-loaded PLGA NPs and Cys-8E NPs (equivalent 5 mg/kg docetaxel) via the tail vein. At a predetermined time point, 40 μL blood was drawn retroorbitally with sodium heparin used as an anticoagulant. Plasma was obtained by 3000 rpm centrifugation for 5 min., then 10 μL plasma was diluted in 10× acidified methanol (0.1% v/v AcOH) overnight. After 10000 rpm centrifugation for 10 min. at 4° C., protein precipitate was removed and its supernatant was analyzed by liquid chromatography (Agilent 1220 system). All control groups were processed using the same procedure. The pharmacokinetic results were calculated by non-compartmental methods using Pksolver (Zhang et al., Computer Methods and Programs in Biomedicine, 2010, 99, 306. The calculated ty2 for PLGA and Cys-8E NPs are 4.79 h and 5.38 h, respectively. FIG. 33 is a plot of the pharmacokinetic profiles obtained for docetaxel-loaded PLGA NPs and Cys-8E NPs.

### Example 18

### In vivo Biocompatibility Study of Cysteine Nanoparticles

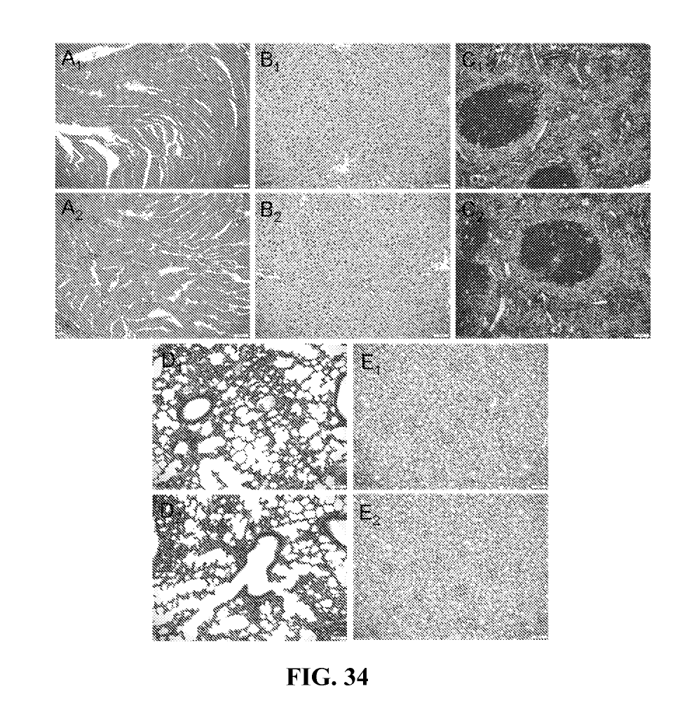

Normal BALB/c female mice were randomly divided into three groups (n=3) and administered daily intravenous injections of NPs made with Cys-8E at 20 mg per kg weight. After three consecutive injections, main organs were collected 2 days post the final injection, fixed with 4% paraformaldehyde, and embedded in paraffin. Tissue sections were stained with hematoxylin-eosin. The results are shown in FIG. 34, which shows images (100× magnification) of histological section of the major organs (A: heart; B: liver; C: spleen; D: lung; E: kidney) of mice given intravenous injections of PBS (A₁-E₁) or Cys-8E NPs (A₂-E₂). Organs of control (PBS) and treated (Cys-8E) animals had similar appearance indicating that the cysteine nanoparticles had good biocompatibility.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1-176. (canceled)

177. A polymer comprising at least one repeating unit according to Formula (I) or Formula (II): 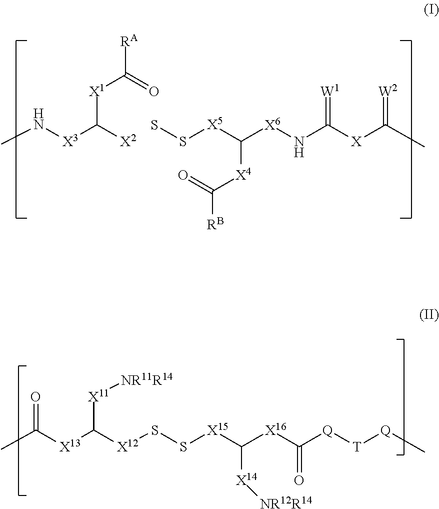
wherein:
X¹is a bond or C₁₋₁₀₀ alkylene;
X² is C₁₋₁₀₀ alkylene;
X³ is a bond or C₁₋₁₀₀ alkylene;
X⁴ is a bond or C₁₋₁₀₀ alkylene;
X⁵ is C₁₋₁₀₀ alkylene;
X⁶ is a bond or C₁₋₁₀₀ alkylene;
R^{A} is OR¹ or NR¹R⁴;
R^{B} is OR² or NR²R⁴;
R¹ is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹ is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
R² is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₁₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
each R³ is independently H, C₁₋₁₀₀ alkyl or C(═O)R⁶;
each R⁴ is independently H or C₁₋₁₀₀ alkyl;
each R⁵ is independently H or C₁₋₁₀₀ alkyl;
each R⁶ is independently H or C₁₋₁₀₀ alkyl;
W¹ is O, S, or NH;
W² is O, S, or NH;
X is C₁₋₁₀₀ alkylene, C₂₋₁₀₀ alkenylene, or C₂₋₁₀₀ alkynylene;
provided that when W¹ and W² are both O, then X is C₃₋₁₀₀ alkylene, C₂₋₁₀₀ alkenylene, or C₂₋₁₀₀ alkynylene;
each m is 0, 1 or 2;
X¹¹ is a bond or C₁₋₁₀₀ alkylene;
X¹² is C₁₋₁₀₀ alkylene;
X¹³ is a bond or C₁₋₁₀₀ alkylene;
X¹⁴ is a bond or C₁₋₁₀₀ alkylene;
X¹⁵ is C₁₋₁₀₀ alkylene;
X¹⁶ is a bond or C₁₋₁₀₀ alkylene
R¹¹ is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹¹ is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;
is H, C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, or 4-10-membered heterocycloalkyl, wherein the C₁₋₁₀₀ alkyl, C₂₋₁₀₀ alkenyl, C₂₋₁₀₀ alkynyl, C₃₋₁₀ cycloalkyl, C₆₋₁₀ aryl, 5-10-membered heteroaryl, and 4-10-membered heterocycloalkyl forming R¹² is optionally substituted with 1, 2, or 3 substituents independently selected from the group consisting of: halo, —CN, OR¹³, NR¹³R¹⁴, —(C═O)R¹⁴, —(C═)OR¹⁴, —(C═O)NR¹⁴R¹⁵, —S(O)ₙR₁₄, and C₆₋₁₀ aryl;
each R¹³ is independently H, C₁₋₁₀₀ alkyl or C(═O)R¹⁶;
each R¹⁴ is independently H or C₁₋₁₀₀ alkyl;
each R¹⁵ is independently H or C₁₋₁₀₀ alkyl;
each R¹⁶ is independently H or C₁₋₁₀₀ alkyl;
each Q is independently O or NR¹⁷;
each R¹⁷ is H or C₁₋₁₀₀ alkyl;
T is C₂₋₁₀₀ alkylene, C₄₋₁₀₀ alkenylene, or C₄₋₁₀₀ alkynylene; and
each n is 0, 1 or 2.

178. The polymer of claim 177 comprising at least one repeating unit according to Formula (I), wherein:
X¹ is a bond or C₁₋₄ alkylene;
X² is C₁₋₄ alkylene;
X³ is a bond or C₁₋₄ alkylene;
X⁴ is a bond or C₁₋₄ alkylene;
X⁵ is C₁₋₄ alkylene;
X⁶ is a bond or C₁₋₄ alkylene;
R^{A} is OR¹ or NR¹R⁴;
R^{B} is OR² or NR²R⁴;
R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming le is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(═O)R⁴, —(═O)OR⁴, —(═O)NR⁴R⁵, —S(O)ₘR⁴, and C₆₋₁₀ aryl;
each R³ is independently H, C₁₋₆ alkyl or C(═O)R⁶;
each R⁴ is independently H or C₁₋₆ alkyl;
each R⁵ is independently H or C₁₋₆ alkyl;
each R⁶ is independently H or C₁₋₆ alkyl;
W¹ is O, S, or NH;
W² is O, S, or NH;
X is C₂₋₂₀ alkylene, C₂₋₂₀ alkenylene, or C₂₋₂₀ alkynylene;
provided that when W¹ and W² are both O, then X is C₃₋₂₀ alkylene, C₂₋₂₀ alkenylene, or C₂₋₂₀ alkynylene; and
each m is 0, 1 or 2.

179. The polymer of claim 177 comprising at least one repeating unit according to Formula (Ia): 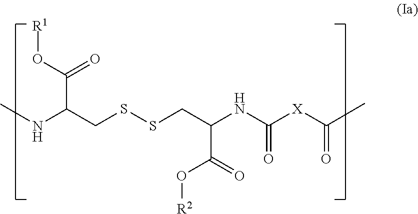
wherein:
R¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming le is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
R² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R² is optionally substituted with 1, 2, or 3 substituents independently selected from the group consisting of: halo, —CN, OR³, NR³R⁴, —(C═O)R⁴, —(C═O)OR⁴, —(C═O)NR⁴R⁵, —S(O)ₘR⁴, and C₆₋₁₀ aryl;
each R³ is independently H, C₁₋₆ alkyl or C(═O)R⁶;
each R⁴ is independently H or C₁₋₆ alkyl;
each R⁵ is independently H or C₁₋₆ alkyl;
each R⁶ is independently H or C₁₋₆ alkyl;
X is C₃₋₂₀ alkylene, C₂₋₂₀ alkenylene, or C₂₋₂₀ alkynylene; and
each m is 0, 1 or 2.

180. The polymer of claim 179, wherein:
R¹ is H, C₁₋₂₀ alkyl, C₂₋₂₀ alkenyl, C₂₋₂₀ alkynyl, C₃₋₁₀ cycloalkyl, or C₆₋₁₀ aryl;
R² is H, C₁₋₂₀ alkyl, C₂₋₂₀ alkenyl, C₂₋₂₀ alkynyl, C₃₋₁₀ cycloalkyl, or C₆₋₁₀ aryl; and
X is C₃₋₂₀ alkylene.

181. The polymer of claim 179, wherein:
R¹ is H or C₁₋₆ alkyl;
R² is H or C₁₋₆ alkyl; and
X is C₄₋₁₀ alkylene.

182. The polymer of claim 179, wherein the at least one repeating unit has the structure selected from:

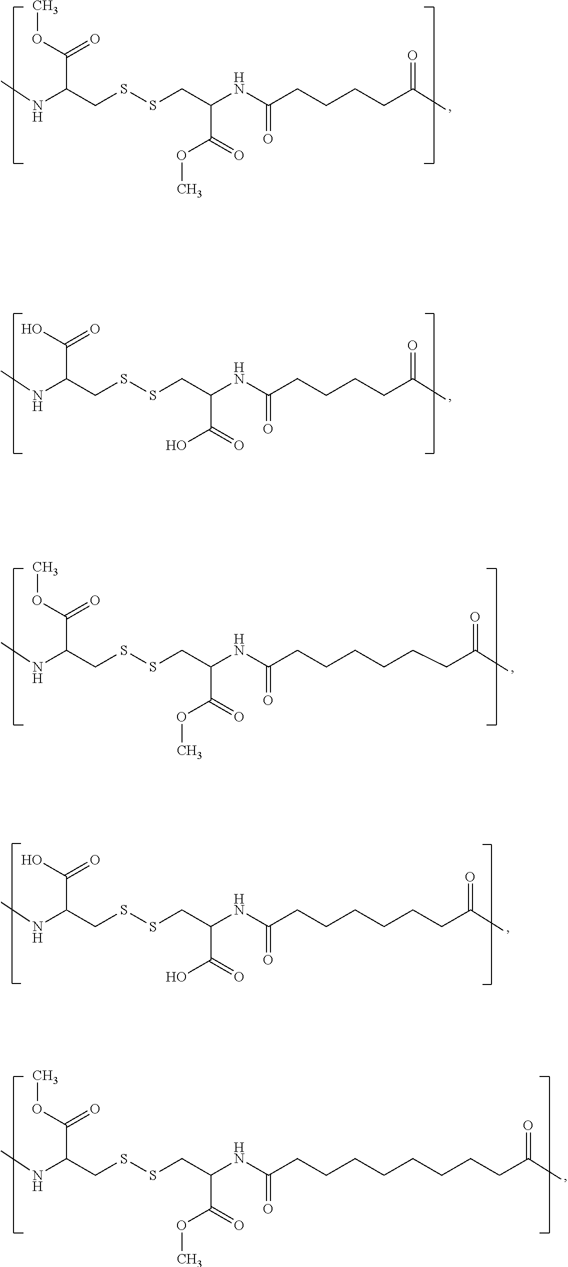

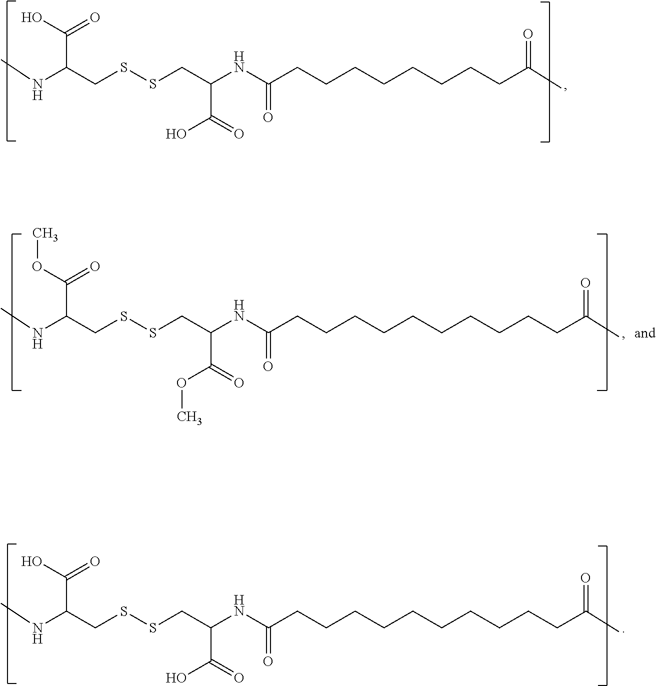

183. The polymer of claim 177 comprising at least one repeating unit according to Formula (II), wherein:
X¹¹ is a bond or C₁₋₄ alkylene;
X¹² is C₁₋₄ alkylene;
X¹³ is a bond or C₁₋₄ alkylene;
X¹⁴ is a bond or C₁₋₄ alkylene;
X¹⁵ is C₁₋₄ alkylene;
X¹⁶ is a bond or C₁₋₄ alkylene;
R¹¹ is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹¹ is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)NR¹⁴R¹⁵, —S(O)ₙR¹⁴, and C₆₋₁₀ aryl;
R¹² is H, C₁₋₂₀ alkyl, C₂₋₂₀ alkenyl, C₂₋₂₀ alkynyl, C₃₋₁₀ cycloalkyl, C₆₋₁₀ aryl, 5-10-membered heteroaryl, or 4-10-membered heterocycloalkyl, wherein the C₁₋₂₀ alkyl, C₁₋₂₀ alkenyl, C₂₋₂₀ alkynyl, C₃₋₁₀ cycloalkyl, C₆₋₁₀ aryl, 5-10-membered heteroaryl, and 4-10-membered heterocycloalkyl forming R¹² is optionally substituted with 1, 2, or 3 substituents independently selected from the group consisting of: halo, —CN, OR¹³, NR¹³R¹⁴, —(C═O)R¹⁴, —(C═O)OR¹⁴, —(C═O)^{NR14}R^{15,} _S(O)ₙR14, and C₆₋₁₀ aryl;
each R¹³ is independently H, C₁₋₆ alkyl or C(═O)R¹⁶;
each R¹⁴ is independently H or C₁₋₆ alkyl;
each R¹⁵ is independently H or C₁₋₆ alkyl;
each R¹⁶ is independently H or C₁₋₆ alkyl;
each Q is independently O or NR¹⁷;
each R¹⁷ is H or C₁₋₆ alkyl;
T is C₂₋₂₀ alkylene, C₄₋₂₀ alkenylene, or C₄₋₂₀ alkynylene; and
each n is 0, 1 or 2.

184. The polymer of claim 183, wherein:
X¹¹ is a bond;
X¹² is C₁₋₄ alkylene;
X¹³ is a bond;
X¹⁴ is a bond;
X¹⁵ is C₁₋₄ alkylene;
X¹⁶ is a bond;
R¹¹ is H, C₁₋₂₀ alkyl, C₂₋₂₀ alkenyl, C₂₋₂₀ alkynyl, C₃₋₁₀ cycloalkyl, or C₆₋₁₀ aryl;
R¹² is H, C₁₋₂₀ alkyl, C₂₋₂₀ alkenyl, C₂₋₂₀ alkynyl, C₃₋₁₀ cycloalkyl, or C₆₋₁₀ aryl;
each Q is 0; and
T is C₄₋₁₀ alkylene.

185. The polymer of claim 183, wherein:
X¹¹ is a bond;
X¹² is CH₂;
X¹³ is a bond;
X¹⁴ is a bond;
X¹⁵ is CH₂;
X¹⁶ is a bond;
R¹¹ is H or C₁₋₆ alkyl;
R¹² is H or C₁₋₆ alkyl;
each Q is O; and
T is (CH₂)₄, (CH₂)₆, (CH₂)₈, or (CH₂)₁₀.

186. A nanoparticle comprising the polymer of claim 177.

187. A composition comprising the polymer of claim 177 and a drug.

188. The composition of claim 187, wherein the drug is encapsulated in the polymer.

189. The composition of claim 188, wherein the drug is covalently attached to the polymer at R¹ or at R².

190. The composition of claim 188, wherein the drug is doxorubicin or docetaxel, or a pharmaceutically acceptable salt thereof.

191. A method of preparing a nanoparticle, the method comprising:
(a) dissolving a polymer of claim 177 in a polar aprotic solvent to form a polymer solution; and
(b) adding the polymer solution to water to provide the nanoparticle.

192. The method of claim 191, further comprising dissolving a drug to form the polymer solution in step (a).

193. The method of claim 192, further comprising adding a stabilizer in step (b).

194. A method of treating cancer in a subject, the method comprising administering to the subject in need thereof a therapeutically effective amount of a composition of claim 190.
